(19) United States
(12) Patent Application Publication
(10) Pub. No.: US 2009/0297494 A1
(43) Pub. Date: Dec. 3, 2009

(54) Diagnostic and treatment of a mental disorder

(76) Inventors: Michel Cuenod, La Tour-de-Peilz (CH); Kim Quang Do-Cuenod, La Tour-de-Peilz (CH); Mirjana Tosic, Lausanne (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/586,071

(22) PCT Filed: Jan. 14, 2005
(86) PCT No.: PCT/EP2005/000337

§ 371 Date: Aug. 17, 2006

Related U.S. Application Data
(60) Provisional application No. 60/536,624, filed on Jan. 15, 2004.
(60) Provisional application No. 60/613,012, filed on Sep. 23, 2004.

(51) Int. Cl. A61K 38/44; C12Q 1/68; C12Q 1/00; C12Q 1/48; G01N 33/68; C07H 21/00; C07K 16/00; A61K 38/53; A61K 31/7052; C40B 20/00; A61P 25/00

(52) U.S. Cl. 424/94.4; 435/6; 435/4; 435/15; 436/89; 536/23.1; 530/387.1; 530/388.1; 436/86; 424/94.5; 514/44 R; 506/2

(57) ABSTRACT

The present invention generally relates to the field of neurological, physiological and psychotic dysfunctions associated with a mental disorder such as schizophrenia, or a predisposition therefor. The invention further relates to genes and proteins, which, when varied in their normal expression, their nucleic acid sequence or in their activity, are associated with the mental disorder. Accordingly, the present invention relates to methods for diagnosis and to methods for prevention and/or treatment of a mental disorder. The present invention additionally relates to compositions for use in diagnosis and to kits for diagnosis of a mental disorder. The invention also relates to the use of a protein or polynucleotide for the manufacture of a medicament for use in the treatment and/or prevention and to a pharmaceutical composition for use in prevention and/or treatment of a mental disorder. Further, the invention relates to methods for screening for a modulator of a mental disorder. More particularly, the present invention relates to methods, compositions and kits, a microarray and reagents for determining the presence of at least one polymorphism and/or at least one combination of polymorphisms of at least one copy of a gene involved in regulating the intracellular glutathione (GSH) level and/or GSH-oxidative stress-related gene expression in a human being. Further, the invention relates to a pharmaceutical composition for use in the treatment and/or prevention of a mental disorder, to the use of an active ingredient such as a protein or polynucleotide for the manufacture of a medicament for use in the treatment and/or prevention of a mental disorder in patients with specific polymorphisms in genes involved in regulating the intracellular GSH level and/or GSH-oxidative stress-related gene expression. The invention also relates to methods of preventing and/or treating a mental disorder comprising administering a medicament to patients having said polymorphisms and to methods of screening for a modulator of a mental disorder.

This application is the National Stage of Application No. PCT/EP2005/000337, filed on Jan. 14, 2005, which claims benefit under 35 U.S.C. § 119(e) of U.S. Provisional Application No. 60/613,012, filed Sep. 23, 2004 which claims benefit under 35 U.S.C. § 119(e) of U.S. Provisional Application No. 60/536,624, filed Jan. 15, 2004. The contents of both are incorporated herein by reference in their entirety.

### FIELD OF THE INVENTION

The present invention generally relates to the field of neurological, physiological and psychotic dysfunctions associated with a mental disorder such as schizophrenia, or a predisposition therefor. The invention further relates to genes and proteins, which, when varied in their normal expression or activity, are associated with the mental disorder. Further the invention relates to genes, which, when varied in their nucleic acid sequence, are associated with said mental disorder. Thus, the present invention relates to methods for diagnosis and to methods for prevention and/or treatment of a mental disorder. The present invention additionally relates to compositions for use in diagnosis and to kits for diagnosis of a mental disorder. The invention also relates to the use of a protein or polynucleotide for the manufacture of a medicament for use in the treatment and/or prevention and to a pharmaceutical composition for use in prevention and/or treatment of a mental disorder. Further, the invention relates to methods for screening for a modulator of a mental disorder.

More particularly, the present invention relates to methods, compositions and kits, a microarray and reagents for determining the presence of at least one polymorphism and/or at least one combination of polymorphisms of at least one copy of a gene involved in regulating the intracellular glutathione (GSH) level and/or GSH-oxidative stress-related gene expression in a human being. Further, the invention relates to a pharmaceutical composition for use in the treatment and/or prevention of a mental disorder, to the use of an active ingredient such as a protein or polynucleotide for the manufacture of a medicament for use in the treatment and/or prevention of a mental disorder in patients with specific polymorphisms in genes involved in regulating the intracellular GSH level and/or GSH-oxidative stress-related gene expression. The invention also relates to methods of preventing and/or treating a mental disorder comprising administering a medicament to patients having said polymorphisms and to methods of screening for a modulator of a mental disorder.

### BACKGROUND OF THE INVENTION

Mental disorders exert a tremendous emotional and economic toll on the patients, their families, and society as a whole. Mental disorders, such as schizophrenic, affective, panic or personality disorders, or manic or psychotic depression are complex and heterogeneous diseases of uncertain etiology that afflict a large percentage of all populations world-wide.

For example Schizophrenia, is an endogenous psychosis characterized by an array of symptoms classically dichotomized into “positive symptoms” (delusions, hallucinations, thought disorder, incoherence of speech and behavior) and “negative symptoms” (deficits in cognitive and social abilities such as social withdrawal, poverty of speech, affective flattening apathy, etc.). The lifetime prevalence is 0.85% in the general population.

Abnormal activity of the neurotransmitter dopamine is one hallmark of schizophrenia. For many years, schizophrenia was treated with classical antipsychotic drugs, the neuroleptics, which include phenothiazines, the butyrophenones, and the thioxanthenes for example. The ability of these drugs to antagonize dopamine receptors correlates with their antipsychotic efficacy. The neuroleptics are effective for treating the positive symptoms of schizophrenia, but have little or no effect on the negative symptoms.

Progress in the treatment of mental disorders has been achieved through the introduction of new, atypical antipsychotic agents. The atypical antipsychotics are a different class of antipsychotic drugs which have a different receptor binding profile and effectiveness against the symptoms of schizophrenia. Atypical antipsychotics bind central serotonin 2(5-HT2) receptors in addition to D2 dopamine receptors. Unlike the neuroleptics, they also improve moderately the negative as well as the positive symptoms. They cause minimal extrapyramidal symptoms and rarely cause tardive dyskinesias, akathisia, or acute dystonic reactions. The first atypical antipsychotic drug approved for the treatment of schizophrenia was clozapine. Clozapine is effective for the treatment of schizophrenia, especially for subjects who do not respond to traditional neuroleptic therapy.

Although the treatment of mental disorders with antipsychotic agents has steadily improved over the years, the causes of schizophrenia are still not understood. Lately, there is increasing evidence in support of an impaired antioxidant defense and increased oxidative injury in schizophrenia. Glutathione (GSH) is the major intracellular non-protein thiol and is known as nucleophilic scavenger and enzyme-catalyzed antioxidant. Reports have emerged that glutathione is depleted in schizophrenia.

Identification of the molecular mechanism underlying a mental disorder would provide a fundamental understanding of the disease process from which a number of clinically Important applications would arise. Thus, there is a need to identify genes and/or proteins involved in or causative for schizophrenia predisposition, onset and/or progression. Said genes or proteins identified may lead to the development of new therapeutics with minimized side effects, diagnostics and/or methods for screening for modulators of a mental disorder.

The inventors show now for the first time that the expression of genes and activity of proteins involved in regulating the intracellular glutathione (GSH) level and/or GSH-oxidative stress-related gene expression as well as plasmatic levels of amino acids are perturbed in patients affected by schizophrenia. A gene expression study for twelve genes involved in the glutathione metabolism using fibroblast cultures obtained from the skin biopsy from schizophrenia patients and a control group showed a significant decrease in two genes directly involved in GSH synthesis: glutamate-cysteine ligase, modifier subunit (GCLM) and glutathione synthetase (GSS). This finding raises the question of whether these reduced levels of mRNAs corresponding to the two genes are due to differences in the genes themselves or to an epigenic factor. A powerful approach to answer this question is to perform an association study in order to detect a possible relationship between polymorphisms in the glutathione-related genes and schizophrenia.

However, until now, no polymorphisms in genes involved in regulating the intracellular glutathione (GSH) level and/or GSH-oxidative stress-related gene expression have been identified which have a clear and significant association with schizophrenia. Thus, there is a need to also identify polymorphisms of genes involved in regulating intracellular glutathione level and/or GSH-oxidative stress-related gene expression and therefore being involved in or causative for schizophrenia predisposition and/or progression. Said identified polymorphisms may lead to the development of new therapeutics with minimal side effects, diagnostics and/or methods for screening for modulators of a mental disorder.

Further, in this study, all the subjects used in the above-mentioned expression study were genotyped in addition and an association study for polymorphisms of genes involved in regulating intracellular glutathione level and/or GSH-oxidative stress-related gene expression was performed. Surprisingly, particular alleles/haplotypes of the glutamate-cysteine ligase modulating subunit (GCLM) gene and the glutathione synthesis (GSS) gene are identified, which have a clear and statistically relevant association with schizophrenia.

### SUMMARY OF THE INVENTION

In a first aspect, the invention provides a method for diagnosis of a mental disorder which includes determining the level of expression of at least one gene involved in regulating the intracellular GSH level. The at least one gene involved in regulating the intracellular GSH level comprises the genes encoding glutamate-cysteine ligase (GCL), preferably glutamate-cysteine ligase modulating subunit (GCLM), glutathione synthetase (GSS), glutathione peroxidase (GPX), preferably GPX1 and/or glutamate/cysteine exchange transporter (system Xc⁻). In another aspect, the invention provides a method for diagnosis of a mental disorder comprising determining the level of activity of at least one protein involved in regulating the intracellular GSH level. According to the invention, said protein includes GCL, preferably GCL catalytic subunit (GCLC), gamma-glutamyltransferase (GGT) and/or system Xc⁻. Another aspect of the invention relates to a method for diagnosing a mental disorder comprising determining the plasmatic level of at least one amino acid. Another aspect of the invention relates to a method for diagnosis of a mental disorder which comprises the determination of intracellular GSH levels. A still further aspect of the invention relates to a method for the diagnosis of a mental disorder or a predisposition therefor in a mammal, particularly in a human being, comprising determining the presence of at least one polymorphism of at least one gene involved in regulating the intracellular glutathione (GSH) level and/or GSH-oxidative stress-related gene expression, wherein said at least one polymorphism is associated with said mental disorder or predisposition therefor.

A further aspect of the invention encompasses a composition for use in diagnosis of a mental disorder which includes at least one oligonucleotide or polynucleotide able to bind to a transcription product of at least one gene involved in regulating intracellular GSH level. According to another aspect of the invention, a composition for use in diagnosis of a mental disorder is provided which comprises at least one antibody, antibody derivative or antibody fragment able to bind at least one protein involved in regulating intracellular GSH level. A yet further aspect of the present invention is a composition for use in diagnosis of a mental disorder comprising at least one means able to determine the activity of at least one protein involved in regulating intracellular GSH level. Another aspect of the invention provides a composition for use in diagnosis of a mental disorder comprising at least one means able to determine the plasmatic level of at least one amino acid.

Other aspects of the present invention provide kits for diagnosis of a mental disorder. In one aspect of the invention such kit comprises a means for determining the level of transcription of at least one gene involved in regulating intracellular GSH level. In a further aspect said kit includes a means for determining the level of protein expressed by at least one gene involved in regulating intracellular GSH level. In yet another aspect the kit for diagnosis of a mental disorder comprises a means for determining the level of activity of the protein expressed by at least one gene involved in regulating intracellular GSH level. In a further aspect, a kit is provided comprising at least one means able to determine the plasmatic level of at least one amino acid.

A still further aspect of the invention relates to a diagnostic composition or kit for the diagnosis of a mental disorder or a predisposition therefor in a mammal, particularly in a human being, comprising at least one primer or probe for determining the presence of at least one polymorphism of at least one gene involved in regulating the intracellular glutathione level and/or GSH-oxidative stress-related gene expression, wherein said at least one polymorphism is associated with a mental disorder or predisposition therefor.

In a further aspect, the present invention is directed to the use of one or more proteins for the manufacture of a medicament for use in the treatment and/or prevention of a mental disorder, wherein the one or more protein is selected from the group consisting of a) GCL, GSS, GPX and system Xc⁻ or a fragment thereof; b) a bioactive protein having a percentage of identity of at least 50% with the amino acid sequence of any one of the proteins of group (a); or c) a bioactive variant of any one of the proteins of group (a) or (b). Another aspect of the invention encompasses the use of one or more polynucleotides for the manufacture of a medicament for use in the treatment and/or prevention of a mental disorder. According to the invention the one or more polynucleotides comprise a sequence encoding one or more proteins as defined above and said sequence or sequences being operatively associated with a tissue specific or a constitutive promoter.

Another aspect of the invention encompasses the use of one or more active ingredients as defined above for the manufacture of a medicament which increases the intracellular GSH level for use in the treatment and/or prevention of a mental disorder in patients having at least one polymorphism and/or at least one combination of polymorphisms of at least one copy of a gene involved in regulating the intracellular glutathione (GSH) level and/or GSH-oxidative stress-related gene expression.

In a still further aspect of the invention, the active ingredient is a protein selected from the group consisting of (a) GCLM and/or GSS or a fragment thereof, (b) a bioactive protein having a percentage of identity of at least 50% with the amino acid sequence of any one of the proteins of a), and (c) a bioactive variant of any one of the proteins of a) or b). In another aspect of the invention, the active ingredient is a polynucleotide comprising a sequence encoding a protein as defined above. In a further aspect, the active ingredient is GSH or a compound increasing the intracellular GSH level.

A further aspect of the invention relates to the use of a compound effective against mental disorders for the manufacture of a medicament for administration to patients having at least one polymorphism and/or at least one combination of polymorphisms of at least one copy of a gene involved in regulating the intracellular glutathione (GSH) level.

Yet another aspect of the present invention is a pharmaceutical composition for use in prevention and/or treatment of a mental disorder comprising one or more protein or one or more polynucleotide as defined above and a pharmaceutically-acceptable carrier.

A further aspect of the invention encompasses a pharmaceutical composition comprising one or more active ingredients which increase the intracellular GSH level and, optionally, a pharmaceutically acceptable carrier, diluent and/or adjuvant for use in the treatment and/or prevention of a mental disorder in patients having at least one polymorphism and/or at least one combination of polymorphisms of at least one copy of a gene involved in regulating the intracellular GSH level and/or GSH-oxidative stress-related gene expression.

Still a further aspect of the invention relates to a microarray for the diagnosis of a mental disorder or a predisposition therefor in a mammal, particularly in a human being, comprising a carrier having immobilised thereto at least one probe for determining the presence of at least one polymorphism and/or of at least one combination of polymorphisms of at least one copy of a gene involved in regulating the intracellular GSH level and/or GSH-oxidative stress-related gene expression, wherein said at least one polymorphism is associated with a mental disorder or a predisposition therefor.

A yet further aspect of the present invention is a primer or probe and/or a combination of primers and/or probes for the diagnosis of a mental disorder or a predisposition therefor in a mammal, particularly in a human being for determining the presence of at least one polymorphism and/or of at least one combination of polymorphisms of at least one copy of a gene involved in regulating the intracellular GSH level and/or GSH-oxidative stress-related gene expression, wherein said at least one polymorphism is associated with a mental disorder or a predisposition therefor.

Still additional aspects of the present invention provide methods for prevention and/or treatment of a mental disorder comprising administering an effective amount of one or more proteins or one or more polynucleotides to a mammal including a human. One aspect provides such method wherein the one or more protein is selected from the group consisting of a) GCL, GSS, GPX and system Xc⁻ or a fragment thereof; b) a bioactive protein having a percentage of identity of at least 50% with the amino acid sequence of any one of the proteins of group (a); or c) a bioactive variant of any one of the proteins of group (a) or (b). In yet another aspect, said effective amount of the one or more polynucleotide comprises a sequence encoding a protein as defined above, and which is operatively associated with a tissue specific or a constitutive promoter. In a further aspect, the invention is directed to a method for prevention and/or treatment of a mental disorder which includes administering an effective amount of an agent that can alter the expression of at least one gene involved in regulating intracellular GSH level. A still additional aspect of the present invention is a method for prevention and/or treatment of a mental disorder which comprises administering an effective amount of an agent that can alter the activity of at least one protein involved in regulating intracellular GSH level. Yet another aspect of the present invention is a method for prevention and/or treatment of a mental disorder which comprises administering an effective amount of an agent that can alter the plasmatic level of at least one amino acid.

Further aspects of the present invention provide a method of preventing and/or treating a mental disorder such as schizophrenia, comprising administering a medicament which is effective against mental disorders and/or increases the intracellular GSH level, to a patient having at least one polymorphism and/or at least one combination of polymorphisms of at least one copy of a gene involved in regulating the intracellular glutathione (GSH) level, and/or GSH-oxidative stress-related gene expression.

In yet other aspects the invention encompasses methods for screening for a modulator of a mental disorder. According to one aspect of the invention, such method comprises the steps (a) determining the level of expression of at least one gene involved in regulating intracellular GSH In a sample of cells; (b) contacting the sample of cells with a candidate agent; (c) determining the level of expression of the at least one gene of step (a) for the sample of cells of step (b); and (d) comparing the levels of expression determined in step (a) and (c), wherein an alteration in the level of expression of the at least one gene indicates that the candidate agent is a modulator of the mental disorder. According to another aspect, such method for screening for a modulator of a mental disorder comprises steps (a) administering a candidate agent to a non-human test animal which is predisposed to be affected or which is affected by a mental disease; (b) administering the candidate agent of step (a) to a matched control non-human animal not predisposed to be affected or being affected by a mental disease; (c) determining the level of expression of at least one gene involved in regulating intracellular GSH in vivo or in vitro in a biological sample isolated from the animal of step (a) and (b); and (d) comparing the levels of expression of step (c); wherein an alteration in the level of expression of the at least one gene indicates that the candidate agent is a modulator of the mental disorder. According to a still further aspect such method for screening for a modulator of a mental disorder includes step (a) determining the level of activity of at least one protein involved in regulating intracellular GSH in a sample of cells; (b) contacting said sample of cells with a candidate agent; and (c) determining the level of activity of the at least one protein of step (a) for the sample of cells of step (b); and (d) comparing the activity determined in step (a) and (c), wherein an alteration in the activity of the at least one protein indicates that the candidate agent is a modulator of the mental disorder. In yet another aspect a method for screening for a modulator of a mental disorder comprises the following steps: (a) administering a candidate agent to a non-human test animal which is predisposed to be affected or which is affected by a mental disease; (b) administering the candidate agent of step (a) to a matched control non-human animal not predisposed to be affected or being affected by a mental disease; (c) determining the levels of activity of at least one protein involved in regulating intracellular GSH in vivo or in vitro in a biological sample isolated from the animal of steps (a) and (b); and (d) comparing the level of activity of step (c); wherein an alteration in the level of activity of the at least one protein indicates that the candidate agent is a modulator of the mental disorder. A still further aspect provides said method for screening for a modulator of a mental disorder which includes step (a) combining at least one protein involved in regulating the intracellular GSH level, the protein binding partner, and a candidate agent to form a reaction mixture; and (b) determining interaction of the protein and the protein binding partner in the presence and absence of the candidate agent. An even further aspect of the invention provides a method for screening for a modulator of a mental disorder which comprises the steps (a) administering a candidate agent to a non-human test animal which is predisposed to be affected or which is affected by a mental disease; (b) administering the candidate agent of (a) to a matched control non-human animal not predisposed to be affected or being affected by a mental disease; (c) determining the level of at least one amino acid in a plasma sample isolated from the animal of steps (a) and (b) and (d) comparing the level of the at least one amino acid of step (c); wherein an alteration in the level of the at least one amino acid indicates that the candidate agent is a modulator of the mental disorder. Finally, a further aspect of the invention relates to a method of screening for a modulator of a mental disorder, comprising determining the effect of a test substance on the activity of at least one copy of a gene involved in regulating the intracellular GSH level and/or GSH-oxidative stress-related gene expression, wherein the at least one copy of a gene has at least one polymorphism and/or at least one combination of polymorphisms which is associated with said mental disorder or predisposition therefor.

Other objects, features, advantages and aspects of the present invention will become apparent to those of skill from the following description. It should be understood, however, that the following description and the specific examples, while indicating preferred embodiments of the invention, are given by way of illustration only. Various changes and modifications within the spirit and scope of the disclosed invention will become readily apparent to those skilled in the art from reading the following description and from reading the other parts of the present disclosure.

### BRIEF DISCUSSION OF THE DRAWINGS

FIG. 1 shows the amino acid sequences of GCLM (NP_{—}002052.1; SEQ ID No: 10), GSS (NP_{—}000169.1; SEQ ID No: 11), GPX1 (SEQ ID No: 12) and of system Xc⁻ (xCT) (SEQ ID No: 13).

FIG. 2 shows the nucleic acid sequence of GCLM (SEQ ID No: 14).

FIG. 3 shows the nucleic acid sequence of GSS (SEQ ID No: 15).

FIG. 4 shows the nucleic acid sequence of GPX1 (SEQ ID No: 16).

FIG. 5 shows the nucleic acid sequence of system Xc⁻ (xCT) (SEQ ID No: 17).

FIG. 6 shows the nucleic acid sequence of GCLM: NT_{—}028050, position 9380597-9403950 (SEQ ID No: 18).

FIG. 7 shows the nucleic acid sequence of GSS: NT_{—}028392, position 1352038-1381802 (SEQ ID No: 19).

FIG. 8 is a diagram showing a positive correlation between GCLM mRNA levels and GSS mRNA levels in fibroblasts isolated from patients with low GCLM gene expression. The amounts of mRNA given represent the relative levels of transcription when compared to a pool of healthy subjects (n=50).

FIG. 9 is a diagram showing a negative correlation of GCLM mRNA levels and positive symptoms in the subgroup of patients with low GCLM gene expression. The amounts of GCLM mRNA given represent the relative levels of transcription when compared to a pool of healthy subjects (n=50) The positive symptoms scores are assessed according to Kay et al. (Kay S R, Opler L A, Fiszbein A, 1986, Positive and negative syndrome sycale (PANSS) manual Multi-Health Systems, Inc, New York).

FIG. 10 is a diagram showing a negative correlation of GCLM mRNA levels and negative symptoms SN5 in the subgroup of patients with low GCLM gene expression. The amounts of GCLM mRNA given represent the relative levels of transcription when compared to a pool of healthy subjects (n=50). The negative symptoms scores are assessed according to Kay et al. 1986 ibid.

FIG. 11 is a diagram showing a negative correlation of GCLM mRNA levels and negative symptoms SN7 in the subgroup of patients with low GCLM gene expression. The amounts of GCLM mRNA given represent the relative levels of transcription when compared to a pool of healthy subjects (n=50). The negative symptoms scores are assessed according to Kay et al. 1986 ibid.

FIG. 12 is a diagram showing a negative correlation of GCLM mRNA levels and general psychopathology in the subgroup of patients with low GCLM gene expression. The amounts of GCLM mRNA given represent the relative levels of transcription when compared to a pool of healthy subjects (n=50). The general psychopathology scores are assessed according to Kay et al. 1986 ibid.

FIG. 13 is a diagram showing a negative correlation of [GSH] levels in blood cells and positive symptoms in the subgroup of patients with low GCLM gene expression. The [GSH] levels are given as μmol per ml. The positive symptoms scores are assessed according to Kay et al. 1986 ibid.

FIG. 14 is a diagram showing a negative correlation between GCL activity and [GSH] levels in blood cells isolated from patients of the subgroup with low GCLM gene expression. The GCL activity as measured in blood cells (RBC) is given in μmol/min/g hemoglobin and the [GSH] levels as measured in blood cells (RBC) are given in μmol per ml blood.

FIG. 15 is a diagram showing the correlation between GCL activity and [GSH] levels in blood cells isolated from control subjects. The GCL activity as measured in blood cells (RBC) is given in μmol/min/g hemoglobin and the [GSH] levels as measured in blood cells (RBC) are given in μmol per ml blood.

FIG. 16 is a diagram showing the correlation between GCL activity and [GSH] levels in blood cells isolated from patients of the subgroup with high GCLM gene expression. The GCL activity as measured in blood cells (RBC) is given in μmol/min/g hemoglobin and the [GSH] levels as measured in blood cells (RBC) are given in μmol per ml blood.

FIG. 17 is a diagram showing a positive correlation between GGT activity and cysteinyl-glycine (Cys-Gly) levels in plasma isolated from control subjects. The GGT activity as measured is given in units/liter and the Cys-Gly levels are given in μmol per liter.

FIG. 18 is a diagram showing the absence of a correlation between GGT activity and cysteinyl-glycine (Cys-Gly) levels in plasma isolated from patients. The GGT activity as measured is given in units/liter and the Cys-Gly levels are given in μmol per liter.

FIG. 19 is a diagram showing a positive correlation between glutamate and cystine levels in plasma isolated from control subjects. The glutamate and cystine levels are given as μmol per liter.

FIG. 20 is a diagram showing the absence of a correlation between glutamate and cystine levels in plasma isolated from the subgroup of patients with low GCLM gene expression. The glutamate and cystine levels are given as μmol per liter.

### DESCRIPTION OF THE INVENTION

All patent applications, patents and literature references cited herein are hereby incorporated by reference in their entirety.

In practicing the present invention, many conventional techniques in molecular biology, microbiology, and recombinant DNA are used. These techniques are well known and are explained in, for example, Current Protocols in Molecular Biology, Volumes I, II, and III, 1997 (F. M. Ausubel ed.); Sambrook et al., 1989, Molecular Cloning: A Laboratory Manual, Second Edition, Cold Spring Harbor Laboratory Press, Cold Spring Harbor, N.Y.; DNA Cloning: A Practical Approach, Volumes I and II, 1985 (D. N. Glover ed.); Oligonucleotide Synthesis, 1984 (M. L. Gait ed.); Nucleic Acid Hybridization, 1985, (Hames and Higgins); Transcription and Translation, 1984 (Hames and Higgins eds.); Animal Cell Culture, 1986 (R. I. Freshney ed.); Immobilized Cells and Enzymes, 1986 (IRL Press); Perbal, 1984, A Practical Guide to Molecular Cloning; the series, Methods in Enzymology (Academic Press, Inc.); Gene Transfer Vectors for Mammalian Cells, 1987 (J. H. Miller and M. P. Calos eds., Cold Spring Harbor Laboratory); and Methods in Enzymology Vol. 154 and Vol. 155 (Wu and Grossman, and Wu, eds., respectively).

The present invention relates to the identification of genes that are expressed at lower levels in a subject affected by a mental disorder such as schizophrenia. The invention also relates to the identification of proteins with altered activity in a patient affected by the mental disorder when compared to a healthy subject or subject population.

By virtue of their low expression and/or altered activity these genes or proteins can be utilized in the diagnosis, disease prevention and treatment, screening for a modulator of the disease or disorder, i.e. screening for agonists and antagonists of the genes or proteins of the invention, and/or post-treatment follow-up of subjects affected or at risk of being affected with a mental disorders which include, but are not limited to schizophrenic disorders (such as schizophreniform or schizoaffective disorders), affective disorders (such as major depressive disorder, bipolar disorder, mood disorder, conduct disorder, Tourette's disorder or tic disorder), psychoactive substance use disorders (such as alcohol withdrawal syndrome), personality disorders, delirium, dementia, epilepsy, panic disorder, obsessive compulsive disorder, intermittent explosive disorder; and impulse control disorder, psychosis (such drug induced or dementia induced psychosis), attention-deficit-hyperactivity disorder (ADHD), and manic or psychotic depression.

The genes of the invention with altered expression pattern include genes involved in regulating the intracellular glutathione (GSH) level and comprise the genes encoding glutamate-cysteine ligase (GCL), preferably GCL modulating subunit (GCLM), glutathione synthetase (GSS), glutathione peroxidase (GPX), preferably GPX1 and/or glutamate/cysteine exchange transporter (system Xc⁻). The proteins of the invention with altered activity include GCL, preferably GCL catalytic subunit (GCLC), gamma-glutamyltransferase (GGT) and/or system Xc⁻.

Any selection of at least one of the gene or protein may be utilized as a marker/diagnostic, therapeutic and/or therapeutic target for the mental disorder. In particularly useful embodiments, at least two, three, four or five of these genes and/or proteins can be selected and their expression and/or activity monitored sequentially or simultaneously to provide expression and/or activity profiles for use in various aspects. For example, expression profiles of the genes and/or activity profiles of the proteins provide valuable molecular tools for rapidly diagnosing and monitoring the predisposition, severity and/or progression of a mental disorder. Changes in the expression and/or activity profile from a baseline profile can further be used as an indication for evaluating drug efficacy.

Furthermore, the invention also relates to the identification of perturbed levels of GSH and/or altered plasmatic levels of amino acids and/or amino acid derivatives in patients affected by the mental disorder. The term “amino acid” as used herein includes but is not limited to naturally occurring amino acids, amino acid derivatives, homologues, analogues, or chemical equivalents thereof, such as phosphothreonine, phosphoserine, homocsysteine, homocystine, homoserine, glycylglycine, dimethylglycine, N-acetyl-glutamate, selenocysteine, cystine, allothreonine, and it also includes molecules consisting of two or three amino acids, such as cysteinylglycine or gamma-glutamylcysteinylglycine. The invention relates in particular, to the identification of altered plasmatic levels and/or correlations of cystine, glutamate, cysteine, homocysteine and/or cysteinyl-glycine.

The determining of GSH levels, preferably in blood alone or together with the determining of plasmatic levels of at least one amino acid or amino acid derivative such as cystine, glutamate, cysteine, homocysteine and/or cysteinyl-glycine each alone, sequentially or simultaneously to the monitoring of gene expression and/or protein activity as described above can be employed in various aspects of the invention.

Accordingly, the invention provides methods for diagnosis of a mental disorder which includes determining the level of expression of at least one gene involved in regulating the intracellular GSH level. Such methods include predicting a predisposition of a subject i.e. the risk of a subject of being affected by a mental disorder such as schizophrenia. It also includes methods for monitoring the progression of a mental disorder in a subject. Additionally, methods of preventing or treating a subject affected or at risk of being affected by a mental disorder such as schizophrenia. Further, also included are methods for the identification of agents that are useful in treating a subject affected by or at risk of being affected by a mental disorder such as schizophrenia, and methods for monitoring the efficacy of certain drug treatments for a mental disorder such as schizophrenia.

The method for diagnosis of a mental disorder comprises determining the level of expression of at least one gene involved in regulating the intracellular GSH level. Any selection of at least one of the gene selected from the genes encoding GCL, preferably GCLM, GSS, GPX, preferably GPX1 and/or system Xc⁻, preferably xCT, may be utilized as a marker for the mental disorder. In particularly useful embodiments, at least two or three of these genes may be selected and their expression measured or monitored either sequentially or simultaneously. Preferably, the level of expression is determined for GCLM and GSS, for GSS and GPX1, or for GCLM and GPX1. Most preferably GCLM, GSS and GPX1 expression are determined.

According to a preferred embodiment of the invention the method further comprises comparing the level of expression determined for a subject with the level of expression of the corresponding at least one gene for a subject or subject population not affected by the mental disorder; wherein a difference of more than 20 percent indicates that the subject is affected or at risk of being affected by the mental disorder. Preferably, a difference of at least about 22, 25, 30, 35, 40, 45, 50, 55, 60 percent in the level of expression is an indication that a subject is affected or at risk of being affected by a mental disorder. Most preferably a decrease in expression is an indication that a subject is affected or at risk of being affected by a mental disorder.

According to another aspect of the invention a method for diagnosis of a mental disorder is provided which comprises determining the level of activity of at least one protein or a fragment thereof involved in regulating the intracellular GSH level. Any selection of at least one of the protein or protein fragment selected from GCL, preferably GCLC, GGT and system Xc⁻, preferably xCT, may be utilized as a marker for the mental disorder. In particularly useful embodiments, the activity of GCL and GGT, or GCL and system Xc⁻, or GGT and system Xc⁻ can be measured. Most preferably, the activity of GCL, GGT and system Xc⁻ are measured. In a preferred embodiment said method further comprises comparing the level of activity of the protein or the fragment thereof determined for a subject with the level of activity of the corresponding protein for a subject or subject population not affected by the mental disorder; and wherein a difference of more than 10 percent indicates that the subject is affected or at risk of being affected by the mental disorder. Preferably, a difference of at least about 12, 15, 20, 22, 25, 30, 35, 40, 45, or 50 percent in the level of activity is an indication that a subject is affected or at risk of being affected by a mental disorder.

Another aspect of the invention relates to a method for diagnosing a mental disorder comprising determining the plasmatic level of at least one amino acid. Particularly preferred is the determination of the plasmatic level of cystine, glutamate, cysteine, homocysteine and/or cysteinyl-glycine. In another preferred embodiment said method further comprises comparing the plasmatic level of amino acids such as cysteine and/or homocysteine of a subject with the levels of a subject or subject population not affected by the mental disorder; wherein a difference of more than 5 percent indicates that the subject is affected or at risk of being affected by the mental disorder. Preferably, a difference of at least about 6, 8, 10, 15, 20, 25, 30, 35, 40, 45 or 50 percent or more in the amount of cysteine and/or homocysteine is an indication that a subject is affected or at risk of being affected by a mental disorder. Most preferably an increase in the plasmatic levels of cysteine and/or homocysteine is an indication that a subject is affected or at risk of being affected by a mental disorder.

Another aspect of the invention relates to a method for diagnosis of a mental disorder which comprises the determination of intracellular GSH levels. Particularly preferred is the determination of the GSH level in the brain or in blood cells. In another preferred embodiment said method further comprises comparing the GSH level of a subject with the GSH level of a subject or subject population not affected by the mental disorder; wherein a difference of more than 5 percent indicates that the subject is affected or at risk of being affected by the mental disorder. Preferably, a difference of at least about 6, 8, 10, 15, 20, 25, 30, 35, 40, 45 or 50 percent or more in the level of GSH is an indication that a subject is affected or at risk of being affected by a mental disorder.

The methods of the invention may be performed in vivo, in vitro or ex vivo. The level of expression of the gene and/or the level of activity of the protein involved in regulating the intracellular GSH level and/or the plasmatic level of the at least one amino acid and/or the level of GSH and/or the presence of at least one polymorphism and/or at least one combination of polymorphisms of at least one copy of a gene involved in regulating the intracellular GSH level and/or GSH-oxidative stress-related gene expression-may be determined in a biological sample taken from a subject to be diagnosed. Such biological sample includes a blood sample or tissue sample. Suitable tissue samples include whole blood, semen, saliva, tears, urine, fecal material, sweat, buccal smears, skin, and biopsies of specific organ tissues, such as muscle, brain or nerve tissue and hair. Most preferably, a suitable tissue sample comprises blood. Tissue samples also include cells and cell types isolated from such biological sample. Most preferably, a suitable tissue sample comprises fibroblasts or neurons. According to the most preferred embodiments of the invention, a biological sample for determining the level of expression or the level of activity of a gene or protein according to the invention comprises fibroblasts, isolated or preferably isolated in cultured; a biological sample for determining the activity of a protein according to the invention such as the activity of GCL or for determining GSH levels comprises blood cells; and a biological sample for determining the levels of amino acids, such as cystine, glutamate, cysteine, homocysteine and/or cysteinyl-glycine or for determining the level of protein activity such as the activity of GGT comprises plasma.

The biological sample may be obtained from the subject, a human or an animal, by known surgical methods, e.g., surgical resection or needle biopsy. A sample obtained from a subject or a subject population not affected by the mental disorder is determined using the same approach as used for the sample obtained from the subject, and may be obtained at the same time as the sample obtained from the subject to be diagnosed, or may be a pre-established control.

In the methods of the invention, the determined level of expression of a gene, activity of a protein, plasmatic level of an amino acid and/or the level of GSH obtained from a subject affected or at risk of being affected preferably differs from the level of expression of the gene, activity of the protein, plasmatic level of the amino acid and/or the level of GSH obtained from a subject or subject population not affected by the mental disorder by a statistically significant amount. In preferred embodiments, at least about a 5 percent difference in the level of expression of the gene, activity of the protein, plasmatic level of the amino acid and/or the level of GSH is an indication that the subject is affected or at risk of being affected by a mental disorder. Preferably, the difference is at least about 6, 8, 10, 15, 20, 25, 30, 35, 40, 45 or 50 percent.

The level of expression of the at least one gene involved in regulating the intracellular GSH level can be detected by measuring the level of transcription of the gene. Methods for measuring the level of transcription of a gene comprise measuring the level of mRNA. RNA can be isolated from the samples by methods well known to those skilled in the art as described, e.g., in Ausubel et al., Current Protocols in Molecular Biology, Vol. 1, pp. 4.1.1-4.2.9 and 4.5.1-4.5.3, John Wiley & Sons, Inc. (1996). Methods for measuring the level of transcription are well known in the art and usually involve hybridization of at least one oligonucleotide or polynucleotide to a transcription product, an mRNA. Such methods include, but are not limited to Northern blot analysis, reverse transcriptase PCR, real-time PCR, RNAse protection and microarray analysis and other hybridization methods.

The oligonucleotide or polynucleotide is preferably of sufficient length to specifically hybridize only to complementary transcripts of the above genes according to the invention. As used herein, the terms “oligonucleotide” or “polynucleotide” refer to a single-stranded nucleic acid. Generally the oligonucleotide or polynucleotide will be at least 16 to 20 nucleotides in length, although in some cases longer probes of at least 20 to 25 nucleotides will be desirable. The oligonucleotide or polynucleotide can be labeled with one or more labeling moieties to permit detection of the hybridized probe/target polynucleotide complexes. Labeling moieties can include compositions that can be detected by spectroscopic, biochemical, photochemical, bioelectronic, immunochemical, electrical optical or chemical means. Examples of labeling moieties include, but are not limited to, radioisotopes, e.g., ³²P, ³³P, ³⁵S, chemiluminescent compounds, labeled binding proteins, heavy metal atoms, spectroscopic markers such as fluorescent markers and dyes, linked enzymes, mass spectrometry tags, and magnetic labels.

A particularly useful method for detecting the level of mRNA transcripts obtained from a plurality of genes involves hybridization of labeled mRNA to an ordered array of oligonucleotides or polynucleotides. Typically, the oligonucleotides or polynucleotides utilized in this hybridization method are bound to a solid support. Examples of solid supports include, but are not limited to, membranes, filters, slides, paper, nylon, wafers, fibers, magnetic or nonmagnetic beads, gels, tubing, polymers, polyvinyl chloride dishes, etc. Any solid surface to which the oligonucleotides or polynucleotides can be bound, either directly or indirectly, either covalently or non-covalently, can be used. Such probe arrays for expression monitoring can be prepared and used according to techniques which are well known to those skilled in the art as described, e.g., in Lockhart et al., Nature Biotechnology, Vol. 14, pp. 1675-1680 (1996); McGall et al., Proc. Natl. Acad. Sci. USA, Vol. 93, pp. 13555-13460 (1996); and U.S. Pat. No. 6,040,138. Such a method allows the level of transcription of a plurality of genes to be measured simultaneously to generate gene expression profiles or patterns. The gene expression profile derived from the biological sample obtained from the subject can be compared with the gene expression profile derived from the sample obtained from a subject or subject population not affected by a mental disorder. Thereby it can be determined whether the subject is affected or is at risk of being affected by the mental disorder such as schizophrenia. According to a preferred embodiment of the invention, the level of expression is determined by using at least one oligonucleotide or polynucleotide as described in Table 1. The level of GCLM transcripts is preferably determined by use of the primer pairs #Hs00157694_ml (Applied Biosystems), and optionally by further use of the probe SEQ ID No: 1, the level of GSS transcripts by use of primer SEQ ID No: 3 and/or 4, and optionally by use of probe SEQ ID No: 2, the level of GPX1 transcripts by use of primer SEQ ID No: 6 and/or 7, and optionally by use of probe SEQ ID No: 5, the level of System Xc⁻4F2 transcripts by use of the primer pairs #Hs00374243 (Applied Biosystems), and optionally by further use of the probe SEQ ID No: 8, and the level of System Xc⁻xCT transcripts by use of the primer pairs #Hs00204928 (Applied Biosystems), and optionally by further use of the probe SEQ ID No: 9.

TABLE 1

Oligonucleotides or polynucleotides
probes and primers for PCR analysis

| | SEQ | | |
|---|---|---|---|
| Gene | ID No | Sequence | Primer/Probe |
| GCLM | 1 | 5′ CACAGCGAGGAGCTTCATGATTGTA 3′ | Probe |
| | | Applied Biosystems assay # | Primer pairs |
| | | Hs00157694_m1 | |
| GSS | 2 | 5′ TGATGGTGCTGGAAAG 3′ | Probe |
| | 3 | 5′ CTGCCTTCCTGGAGCAAACT 3′ | Forward primer |
| | 4 | 5′ CGAGCGGTAAAGTCATCCTGTT 3′ | Reverse primer |
| GPX1 | 5 | 5′ TCTTGGCGTTGTGGTGATGC 3′ | Probe |
| | 6 | 5′ CCCGTGCAACCAGTTTGG 3′ | Forward primer |
| | 7 | 5′ GACGTACTTGAGGGAATTCTGAAT 3′ | Reverse primer |
| System Xc⁻ | 8 | 5′ CAGCTGCCCTTCCTGGACAGCCTAT 3′ | Probe |
| 4F2 | | Applied Biosystems assay # | Primer pairs |
| | | Hs00374243 | |
| System Xc⁻ | 9 | 5′TATGCTGGCTGGTTTTACCTCAACT3′ | Probe |
| xCT | | Applied Biosystems assay # | Primer pairs |
| | | Hs00204928 | |

According to another preferred embodiment of the invention, the level of expression may be determined by measuring the level of protein by the at least one gene involved in regulating the intracellular GSH level. Expression of the at least one protein or a fragment of the protein, e.g., the catalytic domain, can be detected by a probe which is detectably labeled, or which can be subsequently labeled. Generally, the probe can be an antibody, an antibody derivative, or an antibody fragment which is able recognizes the expressed protein. As used herein, the term “antibody” includes, but is not limited to, polyclonal antibodies, monoclonal antibodies, humanized or chimeric antibodies and biologically functional antibody fragments, which are those fragments sufficient for binding of the antibody fragment to the protein or a fragment of the protein. Preferably the GPX1 antibody from Biodesign International (#K90097C) and the GSS antibody from Santa Cruz Biotechnology (#sc-15092) are used in embodiments of the invention.

For the production of antibodies to a protein encoded by one of the disclosed genes or to a fragment of the protein, various host animals may be immunized by injection with the polypeptide, or a portion thereof. Such host animals may include, but are not limited to, rabbits, mice and rats, to name but a few. Various adjuvants may be used to increase the immunological response, depending on the host species, including, but not limited to, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum. Polyclonal antibodies are heterogeneous populations of antibody molecules derived from the sera of animals immunized with an antigen, such as target gene product, or an antigenic functional derivative thereof. For the production of polyclonal antibodies, host animals, such as those described above, may be immunized by injection with the encoded protein, or a portion thereof, supplemented with adjuvants as also described above.

Monoclonal antibodies (mAbs), which are homogeneous populations of antibodies to a particular antigen, may be obtained by any technique which provides for the production of antibody molecules by continuous cell lines in culture. These include, but are not limited to, the hybridoma technique of Kohler and Milstein (Nature, Vol. 256, pp. 495-497 (1975); and U.S. Pat. No. 4,376,110), the human B-cell hybridoma technique (Kosbor et al., Immunology Today, Vol. 4, p. 72 (1983); Cole et al., Proc. Natl. Acad. Sci. USA, Vol. 80, pp. 2026-2030 (1983)), and the EBV-hybridoma technique (Cole et al., Monoclonal Antibodies and Cancer Therapy, Alan R. Liss, Inc., pp. 77-96 (1985)). Such antibodies may be of any immunoglobulin class, including IgG, IgM, IgE, IgA, IgD, and any subclass thereof. The hybridoma producing the mAb of this invention may be cultivated in vitro or in vivo. Production of high titers of mAbs in vivo makes this the presently preferred method of production. In addition, techniques developed for the production of “chimeric antibodies” (Morrison et al., Proc. Natl. Acad. Sci. USA, Vol. 81, pp. 6851-6855 (1984); Neuberger et al., Nature, Vol. 312, pp. 604-608 (1984); Takeda et al., Nature, Vol. 314, pp. 452-454 (1985)) by splicing the genes from a mouse antibody molecule of appropriate antigen specificity, together with genes from a human antibody molecule of appropriate biological activity, can be used. A chimeric antibody is a molecule in which different portions are derived from different animal species, such as those having a variable or hypervariable region derived from a murine mAb and a human immunoglobulin constant region. Alternatively, techniques described for the production of single-chain antibodies (U.S. Pat. No. 4,946,778; Bird, Science, Vol. 242, pp. 423-426 (1988); Huston et al., Proc. Natl. Acad. Sci. USA, Vol. 85, pp. 5879-5883 (1988); and Ward et al., Nature, Vol. 334, pp. 544-546 (1989)) can be adapted to produce differentially expressed gene-single chain antibodies. Single chain antibodies are formed by linking the heavy and light chain fragments of the Fv region via an amino acid bridge, resulting in a single-chain polypeptide. Most preferably, techniques useful for the production of “humanized antibodies” can be adapted to produce antibodies to the proteins, fragments or derivatives thereof. Such techniques are disclosed in U.S. Pat. Nos. 5,932,448; 5,693,762; 5,693,761; 5,585,089; 5,530,101; 5,569,825; 5,625,126; 5,633,425; 5,789,650; 5,661,016; and 5,770,429. Antibody fragments which recognize specific epitopes may be generated by known techniques. For example, such fragments include, but are not limited to, the F(ab′)₂ fragments, which can be produced by pepsin digestion of the antibody molecule, and the Fab fragments, which can be generated by reducing the disulfide bridges of the F(ab′)₂ fragments. Alternatively, Fab expression libraries may be constructed (Huse et al., Science, Vol. 246, pp. 1275-1281 (1989)) to allow rapid and easy identification of monoclonal Fab fragments with the desired specificity.

The level of protein (fragment) expressed in a biological sample may then be determined by immunoassay methods which utilize the antibodies, antibody derivatives, or antibody fragments described above. Such immunoassay methods include, but are not limited to, Western blotting, fluorescence-activated cell sorting (FACS), immunohistochemistry, enzyme-linked immunosorbant assays (ELISA), enzyme linked immuno-spot assay (ELISPOT), dot blotting, competitive and noncompetitive protein binding assays, and other methods commonly used and widely described in scientific and patent literature, and many employed commercially.

Particularly preferred, for ease of detection, is the sandwich ELISA, of which a number of variations exist, all of which are intended to be encompassed by the present invention. For example, in a typical forward assay, unlabeled antibody, antibody derivative or antibody fragment is immobilized on a solid substrate and the sample to be tested is brought into contact with the bound molecule and incubated for a period of time sufficient to allow formation of an antibody-antigen binary complex. At this point, a second antibody, antibody derivative, or antibody fragment labeled with a molecule capable of inducing a detectable signal, is then added and incubated, allowing time sufficient for the formation of a ternary complex of antibody-antigen-labeled antibody. Any unreacted material is washed away, and the presence of the antigen is determined by observation of a signal, or may be quantified by comparing with a control sample containing known amounts of antigen. Variations on the forward assay include the simultaneous assay, in which both sample and antibody are added simultaneously to the bound antibody, or a reverse assay, in which the labeled antibody and sample to be tested are first combined, incubated and added to the unlabeled surface bound antibody. These techniques are well known to those skilled in the art, and the possibility of minor variations will be readily apparent. As used herein, “sandwich assay” is intended to encompass all variations on the basic two-site technique.

The most commonly used reporter molecules for labeling an antibody, antibody fragment or derivative in this type of assay are either enzymes, fluorophore- or radionuclide-containing molecules. In the case of an enzyme immunoassay (EIA), an enzyme is conjugated to the second antibody, usually by means of glutaraldehyde or periodate. As will be readily recognized, however, a wide variety of different ligation techniques exist which are well-known to the skilled artisan. Commonly used enzymes include horseradish peroxidase, glucose oxidase, beta-galactosidase and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used. It is also possible to employ fluorogenic substrates, which yield a fluorescent product, rather than the chromogenic substrates noted above. A solution containing the appropriate substrate is then added to the tertiary complex. The substrate reacts with the enzyme linked to the second antibody, giving a qualitative visual signal, which may be further quantified, usually spectrophotometrically, to give an evaluation of the amount of protein or fragment thereof. Alternately, fluorescent compounds, such as fluorescein and rhodamine, may be chemically coupled to antibodies without altering their binding capacity. When activated by illumination with light of a particular wavelength, the fluorochrome-labeled antibody absorbs the light energy, inducing a state of excitability in the molecule, followed by emission of the light at a characteristic longer wavelength. The emission appears as a characteristic color visually detectable with a light microscope. Immunofluorescence and EIA techniques are both very well established in the art and are particularly preferred for the present method. However, other reporter molecules, such as radioisotopes, chemiluminescent or bioluminescent molecules may also be employed. It will be readily apparent to the skilled artisan how to vary the procedure to suit the required use.

Another aspect of the invention provides a method for diagnosis of a mental disorder, wherein the level of activity of at least one protein involved in regulating the intracellular GSH level is determined. In a preferred embodiment of the invention, the level of activity of GCL, GGT and/or system Xc⁻ is determined. Methods for measuring the activity of GCL, GGT and/or system Xc⁻ are well known to those skilled in the art. GCL activity may for example be determined as described by Gegg et al. (Analytical Biochemistry, 304, 26-32, 2002), and GGT activity may for example be measured determined by measuring the GGT catalyzed formation of 5-amino-2-nitrobenzoate from γ-glutamyl-3-carboxy-4-nitroanilide. Enzyme activity is measured at an absorbance of 405 nm. System Xc⁻ activity may for example be determined by measurement of [³⁵S] cystine uptake, as e.g. described by Reference: Bannai S and Kitamura E (Journal of Biological Chemistry 255, 2372-2376, 1980).

In other preferred embodiments of the invention the level of expression of at least one gene involved in regulating the intracellular GSH level is determined together with the level of activity of at least one protein involved in regulating the intracellular GSH level. The level of expression and/or the level of activity are determined according to the methods of the invention. It is sometimes desirable to determine the level of expression and/or activity of 2, 3, 4 or 5 of those genes and/or proteins. Preferably the level of expression is determined for the gene encoding GCL, most preferably for GCLM, GPX, most preferably for GPX1, GSS and/or system Xc⁻, most preferably for xCT and the level of activity is preferably determined for GCL, most preferably for GCLC, GGT and/or system Xc⁻, most preferably for xCT.

A further aspect of the invention provides a method for diagnosis of a mental disorder, which method comprises determining the plasmatic level of at least one amino acid or determining the intracellular GSH level. In a preferred embodiment of the invention, the plasmatic level of cystine, glutamate, cysteine, homocysteine and/or cysteinyl-glycine is determined. Methods for measuring the level of amino acids in plasma are well known to those skilled in the art, e.g. the following free amino acids may be quantified in plasma as described in Slocum et al. (In “Techniques in diagnostic human Biochemical Genetics”. Hommes Edt. 1991, pp 87-126): Taurine (Tau), Aspartic acid (Asp), Hydroxyproline (Hyp), Threonine (Thr), Serine (Ser), Asparagine (Asn), Glutamic acid (Glu), Glutamine (Gln), Proline (Pro), Glycine (Gly), Alanine (Ala), Citrulline (Cit), Aminobutyric acid (Abu), Valine (Val), Cystine (Cyt), Methionine (Met), Isoleucine (Ile), Leucine (Leu), Tyrosine (Tyr), Phenylalanine (Phe), Ornithine (Orn), Lysine (Lys), 1-CH₃-Histidine (1-CH₃-His), Histidine (His), 3-CH₃-Histidine (3-CH₃-His), Arginine (Arg). Thiol containing amino acids and peptides may be quantified in plasma as described in Jacobsen et al. (Gen Clin Chim, 873-881, 1994).

Another preferred embodiments of a method according to the invention provides further the determining of intracellular GSH levels. Most preferably a method is provided, wherein the level of expression of the GCLM gene, the level of activity of GCL and the intracellular level of GSH are determined, and wherein a decreased expression of the GCLM gene and a negative correlation between GCL activity and GSH levels indicates that the subject is affected or at risk of being affected by the mental disorder. Most preferably the GCLM gene expression is determined for fibroblasts including cultured fibroblasts and the GCL activity and the GSH levels for blood cells isolated from a subject. According to another preferred method of the invention, the level of expression of at least one gene involved in regulating the intracellular GSH level, preferably of GCLM, and the intracellular level of GSH, preferably in blood cells, are determined. The level of expression of GCLM is most preferably determined in fibroblasts isolated from a subject. Most preferably, a decreased expression of GCLM and a decrease in GSH levels indicates that a subject is affected or at risk of being affected by the mental disorder.

A further embodiment of a method according to invention comprises determining the level of expression of at least one gene involved in regulating the intracellular GSH level and determining the plasmatic level of at least one amino acid. Preferably said at least one gene comprises GCL, most preferably GCLM, GPX, most preferably GPX1, GSS and/or system Xc⁻ gene, most preferably xCT, and wherein the at least one amino acid comprises cystine, glutamate, cysteine, homocysteine and/or cysteinyl-glycine. Most preferably a method is provided, wherein the level of expression is determined for GCLM, preferably in fibroblasts isolated from a subject and subsequently cultured, the plasmatic levels of cystine and glutamate are determined, and wherein a decreased expression of GCLM and an absence of correlation between cystine and glutamate levels indicates that a subject is affected or at risk of being affected by the mental disorder.

A yet further embodiment of a method according to invention comprises determining the level of activity of at least one protein involved in regulating the intracellular GSH level and determining the plasmatic level of at least one amino acid. Preferably said at least one protein comprises GCL, most preferably GCLC, GGT and/or system Xc⁻, most preferably xCT, and the at least one amino acid comprises cystine, glutamate, cysteine, homocysteine and/or cysteinyl-glycine. Most preferably a method is provided, wherein the level of activity is determined for GGT, preferably in plasma, and the plasmatic level of cysteinyl-glycine is determined, and wherein an absence of correlation of GGT activity and the level of cysteinyl-glycine indicates that a subject is affected or at risk of being affected by the mental disorder.

The methods of the invention as described above may be used for predicting a predisposition of a subject, i.e. determining the risk of a subject of being affected by a mental disorder such as schizophrenia. The methods may also be used for monitoring the progression of a mental disorder in a subject. Alternatively the methods may be used to monitor the efficacy of a therapeutic agent for a patient in the treatment of a mental disorder such as schizophrenia. Preferably, the level of expression and/or the level of activity of the genes and/or proteins of the invention is determined for more than one gene or protein of the invention. Most preferably, the expression of two or three different genes and/or the level of activity of two or three different proteins of the invention are determined. Said measurement may be performed simultaneously or subsequently. Alternatively or additionally, plasmatic level of at least one amino acid, in particular of cystine, glutamate, cysteine, homocysteine and/or cysteinyl-glycine and/or intracellular levels of GSH may also be determined sequentially or simultaneously to the monitoring of gene expression and/or protein activity according to the methods of the invention.

Another aspect of the invention relates to a method for the diagnosis of a mental disorder or a predisposition therefor in a mammal, particularly in a human being, comprising determining the presence of at least one polymorphism of at least one gene involved in regulating the intracellular glutathione (GSH) level and/or GSH-oxidative stress-related gene expression, wherein said at least one polymorphism is associated with said mental disorder or predisposition therefor. In another embodiment of the invention said method further comprises the determination of the level of expression of the gene and/or the level of activity of the protein involved in regulating the intracellular GSH level and/or the plasmatic level of the at least one amino acid and/or the level of GSH as described above.

The present invention discloses for the first time polymorphisms/haplotypes in glutathione-related genes which have a statistically significant association with schizophrenia. A statistically significant association is preferably an association of a polymorphism/haplotype with the occurrence of the disorder of preferably p<0.05, more preferably p<0.01, still more preferably p<0.001 and most preferably p<0.0001 and/or of preferably an odds ratio (OR)>1.0, more preferably >1.5, still more preferably >3.0 and most preferably >8.0. The determination of significance may be carried out by a polymorphism/haplotype analysis of a sufficient number, e.g. of at least 40, preferably of 40 to 60 schizophrenic patients and of at least 80, preferably of 80 to 120 normal controls. More preferably, the determination of significance may be carried out as described in the Example section. The polymorphisms are preferably single nucleotide polymorphisms (SNPs).

Preferably, the at least one gene involved in regulating the intracellular GSH level and/or GSH-oxidative stress-related gene expression is selected from a glutamate-cysteine ligase, modifier subunit gene (GCLM) and/or a glutathione synthetase gene (GSS). According to a preferred embodiment of the invention, said polymorphism is associated with low expression levels of at least one gene involved in regulating the intracellular glutathione level and/or GSH-oxidative stress-related gene expression, as low level mRNAs of the above-mentioned genes were observed in schizophrenic patients.

Surprisingly, it was found that polymorphisms associated with schizophrenia may occur in introns, the 3′ region and/or the 5′ region of the GCLM and/or GSS gene, e.g. In intron 1 and/or 6 and/or the 3′ region of the GCLM gene, and/or in intron 1, 3, 5, 8, 9 and/or 12, the 3′ region and/or the 5′ region of the GSS gene.

In a preferred embodiment of the invention, the method for the diagnosis of a mental disorder or a predisposition therefor comprises determining a single polymorphism in a chromosomal copy of said genes and/or in two chromosomal copies of said genes, a combination of polymorphisms in a chromosomal copy of said genes and/or in two chromosomal copies of said genes and/or a combination of polymorphisms in at least one chromosomal copy of a combination of the GCLM and the GSS gene, wherein said polymorphism and/or combination of polymorphisms is associated with said mental disorder or predisposition therefor.

Preferably, the polymorphism of the GLCM gene is selected from the group consisting of (a) the polymorphisms rs2235971, rs3170633, rs2064764, rs769211, rs718873, rs718875, rs2301022, (b) polymorphisms being in linkage disequilibrium with at least one of the polymorphisms of (a), and (c) combinations of polymorphisms of (a) and/or (b). More preferably, said polymorphism is rs2235971, rs3170633, rs769211 and/or rs2301022. Most preferably the polymorphism is rs3170633. In a preferred embodiment, the genotype of the polymorphism rs3170633 is selected from the group consisting of the nucleotides AA, AG and/or GG. More preferably, the genotype is GG because individuals having the GG genotype have an approximately three times higher risk of being ill than other individuals.

Preferably, a combination of polymorphisms in at least one chromosomal copy of the GCLM gene is selected from the group consisting of (a) the polymorphisms rs2235971, rs3170633, rs769211 and rs2301022, (b) polymorphisms being in linkage disequilibrium with at least one of the polymorphisms of (a), and (c) combinations of polymorphisms of (a) and/or (b).

Preferably, the combination comprises at least one polymorphism, more preferably at least two polymorphisms and most preferably four polymorphisms selected from the group consisting of (a) the polymorphisms rs2235971, rs3170633, rs769211 and rs2301022, (b) polymorphisms being in linkage disequilibrium with at least one of the polymorphisms of (a), and (c) combinations of polymorphisms of (a) and/or (b).

Preferably, the presence of the nucleotides G for rs2235971, G for rs3170633, G for rs769211 and A for rs2301022 is associated with said mental disorder or predisposition therefor.

Preferably, the polymorphism of the GSS gene is selected from the group consisting of (a) the polymorphisms rs3746450, rs725521, rs1801310, rs2236270, rs2236271, rs2273684, rs734111, rs2025096, rs3761144, (b) polymorphisms being in linkage disequilibrium with at least one of the polymorphisms of (a), and (c) combinations of polymorphisms of (a) and/or (b). More preferably said polymorphism is rs2236270, rs2273684, rs734111, rs2025096 and/or rs3761144. Most preferably, the polymorphism is rs3761144.

In a preferred embodiment, a combination of polymorphisms in at least one chromosomal copy of the GSS gene is selected from the group consisting of (a) the polymorphisms rs2236270, rs2273684, rs734111, rs2025096 and rs3761144, (b) polymorphisms being in linkage disequilibrium with at least one of the polymorphisms of (a), and (c) combinations of polymorphisms of (a) and/or (b).

Preferably, the combination comprises at least one polymorphism, more preferably at least three polymorphisms and most preferably five polymorphisms selected from the group consisting of rs2236270, rs2273684, rs734111, rs2025096 and rs3761144. Preferably the presence of the nucleotides C for rs2236270, T for rs2273684, C for rs734111, G for rs2025096 and C for rs3761144 and/or the presence of the nucleotides T for rs 2236270, G for rs2273684, A for rs734111, G for rs2025096 and G for rs3761144 is associated with said mental disorder or predisposition therefor.

In a preferred embodiment, the combination of polymorphisms in at least one chromosomal copy of the GSS gene and the GCLM gene is selected from the group consisting of (a) the polymorphisms rs2235971, rs3170633, rs769211, rs2301022, rs2236270, rs2273684, rs734111, rs2025096 and/or 3761144, (b) polymorphisms being in linkage disequilibrium with at least one of the polymorphisms of (a), and (c) combinations of polymorphisms of (a) and/or (b), because for these nine polymorphisms in the genes GCLM and GSS jointly, haplotype frequencies were significantly different between patients and controls (p=0.000062). The haplotype with the strongest effect is observed for the combined haplotypes GGGA (GCLM gene) with CTCGC (GSS gene).

In another preferred embodiment, a combination of polymorphisms, so-called haplotypes in two chromosomal copies of the GCLM gene or the GSS gene is homozygous. More preferably, said haplotypes being in a homozygous state are the haplotypes GGGA of the GCLM gene and TGAGG of the GSS gene.

All information concerning the above-mentioned genes are accessible in the http://www.ncbi.nlm.gov and/or http://www-dsi-univ-paris5.fr/genatlas/databases. With regard to the nomenclature of the polymorphisms, reference is made to GCLM at Chr 1p22.1:NT_{—}028050, position 9380597-9403950) glutamate-cysteine ligase, modifier subunit (SEQ-ID No. 18), NT #=Reference Sequence Number, and to GSS at Chr 20q11.1: NT_{—}028392, position 1352038-1381802, glutathione synthetase (SEQ-ID No. 19).

All of the above-mentioned single nucleotide polymorphisms (SNP) were selected from the publicly available databases SNP Consortium (http:/snp.cshl.org) and NCBI (http:/ww.ncbi.nlm.nih.gov). NCBI-annotated SNP numbers, their alternative alleles and their positions and distances from the beginning of the genes are shown in Table 2. The SNP positions are based on the information found in the NCBI graphic representation of the appropriate contig.

TABLE 2

| | | | | SNP-location | |
|---|---|---|---|---|---|
| Gene | Location | SNP# | Allele | in the gene | SNP position |
| GCLM | 1p22.1 | rs2235971 | G/A | 30250 | 3′ region |
| | | rs3170633 | A/G | 23638 | 3′ region |
| | | rs2064764 | A/G | 16623 | intron 6 |
| | | rs769211 | T/G | 14859 | intron 6 |
| | | rs718873 | T/C | 3057 | intron 1 |
| | | rs718875 | T/C | 2946 | intron 1 |
| | | rs2301022 | A/G | 2088 | intron 1 |
| GSS | 20q11.2 | rs3746450 | C/A | 35013 | 3′ region |
| | | rs725521 | T/C | 27590 | 3′ region |
| | | rs1801310 | A/G | 26587 | intron 12 |
| | | rs2236270 | T/G | 20446 | intron 9 |
| | | rs3746450 | C/A | 35013 | 3′ region |
| | | rs2236271 | A/C | 19761 | intron 8 |
| | | rs2273684 | T/G | 13835 | intron 5 |
| | | rs734111 | C/A | 9865 | intron 3 |
| GSS | 20q11.2 | rs2025096 | A/G | 3601 | intron 1 |
| | | rs3761144 | C/G | −474 | 5′ region |

The presence of at least one polymorphism associated with schizophrenia is preferably determined by a genotyping analysis as indicated above. For example, the determination may comprise the analysis of a single polymorphism or of a plurality of polymorphisms in one or two copies of a single gene or in one or two copies of different genes.

The determination may comprise the use of polymorphism-specific primers capable of hybridizing with the respective gene and allowing a discrimination between polymorphisms, particularly SNPs at a predetermined position. For example, the genotyping analysis may comprise a primer extension reaction using polymorphism-specific primers as described in the Example. The determination of individual polymorphisms may be carried out by mass-spectrometric analysis as described in the Example.

A further preferred embodiment comprises a microarray analysis which is particularly suitable for the parallel determination of several polymorphisms. Suitable microarray devices are commercially available.

The subjects to be tested are mammals, preferably human beings, suspected to be suffering from a mental disorder which includes, but is not limited to, schizophrenic disorders (such as schizophreniform or schizoaffective disorders), affective disorders (such as major depressive disorder, bipolar disorder, mood disorder, conduct disorder, Tourette's disorder or tic disorder), psychoaffective substance use disorders (such as alcohol withdrawal syndrome), personality disorders, delerium, dementia, epilepsy, panic disorder, obsessive compulsive disorder, intermittent explosive disorder, impulse control disorder, psychosis (such as drug-induced or dementia-induced psychosis), attention-deficit-hyperactivity disorder (ADHD), and manic or psychotic depression.

As used herein the term “mental disorder” shall mean any pathologic psychological condition, and includes, but is not limited to the following; (also see, Diagnostic and Statistical Manual of Mental Disorders 4^{th} Edition (DSM-IV) Francis A editor, American Psychiatric Press, Wash., D.C., 1994).

### Schizophrenic Disorders

Schizophrenia, Catatonic, Subchronic, (295.21); Schizophrenia, Catatonic, Chronic (295.22); Schizophrenia, Catatonic, Subchronic with Acute Exacerbation (295.23); Schizophrenia, Catatonic, Chronic with Acute Exacerbation (295.24); Schizophrenia, Catatonic, in Remission (295.55); Schizophrenia, Catatonic, Unspecified (295.20); Schizophrenia, Disorganized, Subchronic (295.11); Schizophrenia, Disorganized, Chronic (295.12); Schizophrenia, Disorganized, Subchronic with Acute Exacerbation (295.13); Schizophrenia, Disorganized, Chronic with Acute Exacerbation (295.14); Schizophrenia, Disorganized, in Remission (295.15); Schizophrenia, Disorganized, Unspecified (295.10); Schizophrenia, Paranoid, Subchronic (295.31); Schizophrenia, Paranoid, Chronic (295.32); Schizophrenia, Paranoid, Subchronic with Acute Exacerbation (295.33); Schizophrenia, Paranoid, Chronic with Acute Exacerbation (295.34); Schizophrenia, Paranoid, in Remission (295.35); Schizophrenia, Paranoid, Unspecified (295.30); Schizophrenia, Undifferentiated, Subchronic (295.91); Schizophrenia, Undifferentiated, Chronic (295.92); Schizophrenia, Undifferentiated, Subchronic with Acute Exacerbation (295.93); Schizophrenia, Undifferentiated, Chronic with Acute Exacerbation (295.94); Schizophrenia, Undifferentiated, in Remission (295.95); Schizophrenia, Undifferentiated, Unspecified (295.90); Schizophrenia, Residual, Subchronic (295.61); Schizophrenia, Residual, Chronic (295.62); Schizophrenia, Residual, Subchronic with Acute Exacerbation (295.63); Schizophrenia, Residual, Chronic with Acute Exacerbation (295.94); Schizophrenia, Residual, in Remission (295.65); Schizophrenia, Residual, Unspecified (295.60); Delusional (Paranoid) Disorder (297.10); Brief Reactive Psychosis (298.80); Schizophreniform Disorder (295.40); Schizoaffective Disorder (295.70); Induced Psychotic Disorder (297.30); Psychotic Disorder NOS (Atypical Psychosis) (298.90).

### Affective Disorders

Major Depressive Disorder; Severe with Psychotic Features (296.33); Bipolar I Disorder, Single Manic Episode, Severe with Psychotic Features (296.23); Bipolar I Disorder, Most Recent Episode Hypomanic (296.43); Bipolar I Disorder, Most Recent Episode Manic, Severe with Psychotic Features (296.43); Bipolar I Disorder, Most Recent Episode Mixed, Severe with Psychotic Features (296.63); Bipolar I Disorder Most Recent Episode Depressed, Severe with Psychotic Features (296.53); Bipolar I Disorder, Most Recent Episode Unspecified (296.89); Bipolar II Disorder (296.89); Cyclothymic Disorder (301.13); Bipolar Disorder NOS (366); Mood Disorder Due To (General Medical Condition) (293.83); Mood Disorder NOS (296.90); Conduct Disorder, Solitary Aggressive Type (312.00); Conduct Disorder, Undifferentiated Type (312.90); Tourette's Disorder (307.23), Chronic Motor Or Vocal Tic Disorder (307.22); Transient Tic Disorder (307.21); Tic Disorder NOS (307.20).

### Psychoactive Substance Use Disorders

Alcohol Withdrawal Delirium (291.00); Alcohol Hallucinosis (291.30); Alcohol Dementia Associated with Alcoholism (291.20); Amphetamine or Similarly Acting Sympathomimetic Intoxication (305.70); Amphetamine or Similarly Acting Sympathomimetic Delirium (292.81); Amphetamine or Similarly Acting Sympathomimetic Delusional Disorder (292.11); Cannabis Delusional Disorder (292.11); Cocaine Intoxication (305.60); Cocaine Delirium (292.81); Cocaine Delusional Disorder (292.11); Hallucinogen Hallucinosis (305.30); Hallucinogen Delusional Disorder (292.11); Hallucinogen Mood Disorder (292.84); Hallucinogen Post hallucinogen Perception Disorder (292.89); Phencyclidine (PCP) or Similarly Acting Arylcyclohexylamine Intoxication (305.90); Phencyclidine (PCP) or Similarly Acting Arylcyclohexylamine Delirium (292.81); Phencyclidine (PCP) or Similarly Acting Arylcyclohexylamine Delusional Disorder (292.11); Phencyclidine (PCP) or Similarly Acting Arylcyclohexylamine Mood Disorder (292.84); Phencyclidine (PCP) or Similarly Acting Arylcyclohexylamine Organic Mental Disorder NOS (292.90); Other or Unspecified Psychoactive Substance Intoxication (305.90); Other or Unspecified Psychoactive Substance Delirium (292.81); Other or Unspecified Psychoactive Substance Dementia (292.82); Other or Unspecified Psychoactive Substance Delusional Disorder (292.11); Other or Unspecified Psychoactive Substance Hallucinosis (292.12); Other or Unspecified Psychoactive Substance Mood Disorder (292.84); Other or Unspecified Psychoactive Substance Anxiety Disorder (292.89); Other or Unspecified Psychoactive Substance Personality Disorder (292.89); Other or Unspecified Psychoactive Substance Organic Mental Disorder NOS (292.90).

### Personality Disorders

Personality Disorder, Paranoid (301.00); Personality Disorder, Schizoid (301.20); Personality Disorder, Schizotypal (301.22); Personality Disorder, Antisocial (301.70); Personality Disorder, Borderline (301.83).

Delirium (293.00); Dementia (294.10); Obsessive Compulsive Disorder (300.30); Intermittent Explosive Disorder (312.34); and Impulse Control Disorder NOS (312.39).

In a preferred embodiment, the mental disorder is schizophrenia. Schizophrenia is a severe mental disorder characterized by a variety of signs and symptoms. However, no single symptom is definitive for diagnosis. Rather, diagnosis encompasses a pattern of signs and symptoms, in conjunction with impaired occupational or social functioning (DSM-IV). According to the invention, the term “schizoprenia” is preferably used in the sense of, but not limited to, the criteria for diagnosing schizophrenia from the Amercian Psychiatric Association's Diagnostic and Statistical Manual of Mental Disorders (DSM), in the most recent version DSM-IV. To be diagnosed as having schizophrenia, a person must display:

(A). Characteristic symptoms: two or more of the following, each present for a significant portion of time during a one month period (or less, if successfully treated): delusions, hallucinations, disorganized speech (e.g. frequent derailment or incoherence), grossly disorganized or catatonic behavior, negative symptoms, i.e. affective flattening (lack or decline in emotional response), alogia (lack or decline in speech) or avolition (lack or decline in motivation). Only one Criterion A symptom is required if delusions are bizarre or hallucinations consist of hearing voices.

(B). Social/Occupational dysfunction: for a significant portion of the time since the onset of the disturbance, one or more major areas of functioning such as work, interpersonal relations, or self-care are markedly below the level achieved prior to the onset.

(C). Duration: continuous signs of the disturbance persist for at least six months. The six month period must include at least one month of symptoms (or less, if successfully treated) that meet Criterion A.

The DSM-IV contains five sub-classifications of schizophrenia. These are catatonic type (where marked absences of peculiarities of movement are present), disorganised type (where thought disorder and flat or inappropriate affects are present together), paranoid type (where delusions and hallucinations are present but thought disorder, disorganised behaviour and affective flattening is absent), residual type (where positive symptoms are present at low intensity only) and undifferentiated type (psychotic symptoms are present but the criteria for paranoid, disorganised, or catatonic types have not been met).

Symptoms may also be described as “positive symptoms” (those additional to normal experience and behaviour) and negative symptoms (the lack or decline in normal experience or behavior). “Positive symptoms” describe psychosis and typically include delusions, hallucinations and thought disorder. “Negative symptoms” describe inappropriate or nonpresent emotion, poverty of speech and lack of motivation.

The diagnosis of a mental disorder in a human being can be made based on the results of polymorphism/haplotype determination. The patient to be tested may have one or a plurality of polymorphisms and/or at least one combination of polymorphisms of at least one genomic copy which are associated with a mental disorder, preferably schizophrenia. If such a diagnosis is given, the patient is at a higher risk of developing a mental disorder, preferably schizophrenia.

A further aspect of the invention encompasses a composition for use in diagnosis of a mental disorder which includes at least one oligonucleotide or polynucleotide able to bind to a transcription product of at least one gene involved in regulating intracellular GSH level. Preferably the oligonucleotide or polynucleotide is able to bind to a transcription product of the GCL, preferably GCLM, GSS, GPX, preferably GPX1 and/or system Xc⁻, preferably xCT, gene. Another preferred embodiment encompasses a composition comprising at least one oligonucleotide or polynucleotide selected from Table 1. Such composition may comprise primer pairs #Hs00157694_m1 (Applied Biosystems), and optionally SEQ ID No: 1 able to bind to the transcription product of GCLM gene, or it may comprise the SEQ ID No: 3 and/or 4, and optionally also SEQ ID No: 2 able to bind to the transcription product of the GSS gene, or said composition may comprise SEQ ID No: 6 and/or 7, and optionally SEQ ID No: 5 able to bind to the transcription product of GPX1 gene, or the composition may comprise primer pairs #Hs00374243 (Applied Biosystems), and optionally SEQ ID No: 8 able to bind to the transcription product of system Xc⁻4F2 gene, or it may comprise primer pairs #Hs00204928 (Applied Biosystems), and optionally SEQ ID No: 9 able to bind to the transcription product of Xc⁻xCT gene. The composition comprises preferably the Applied Biosystems assay #Hs00157694_m1 together with SEQ ID No: 3 and SEQ ID No: 4, and optionally SEQ ID No: 1 and SEQ ID No: 2 able to bind to the transcription products of the GCLM and GSS genes.

According to another aspect of the invention, a composition for use in diagnosis of a mental disorder is provided which comprises at least one antibody, antibody derivative or antibody fragment able to bind at least one protein involved in regulating intracellular GSH level. Preferably said compositing comprises a monoclonal antibody as described in detail further above.

A yet further aspect of the present invention is a composition for use in diagnosis of a mental disorder comprising at least one means able to determine the activity of at least one protein involved in regulating intracellular GSH level as described in detail further above. According to a preferred embodiment of the invention the means are able to determine the activity of GCL, preferably of GCLC, GGT and/or system Xc⁻. Preferably, the means for determining the activity of GCL comprise a scintillation analyzer able to determine the amount of ¹⁴C-γ-glutamyl-aminobutyric acid, the means to determining the activity of GGT comprise spectroscopic means able to measure the formation of 5-amino-2-nitrobenzoate, and the means to determine the activity of system Xc⁻ are able to measure [³⁵S] cystine uptake.

Other aspects of the present invention provide kits for diagnosis of a mental disorder. In one aspect of the invention such kit comprises a means for determining the level of transcription of at least one gene involved in regulating intracellular GSH level. In a preferred embodiment of the invention the means for determining the level of transcription comprise at least one oligonucleotide or polynucleotide able to bind to a transcription product of the GCL, preferably GCLM, GSS, GPX, preferably GPX1, and/or system Xc⁻, preferably cXT, gene. Preferably, said oligonucleotide or polynucleotide comprises a sequence selected from Table 1. In another preferred embodiment, said kit comprises oligonucleotides or polynucleotides able to bind to the transcription products of the GCLM and GSS genes, or to the transcription products of the GCLM and GPX1 genes, or to the transcription products of the GSS and GPX1, or most preferably to the transcription products of the GCLM, GSS and GPX1 genes. Alternatively, said kit comprises oligonucleotides or polynucleotides able to bind to the transcription products of the GCLM and system Xc⁻, GSS and system Xc⁻, GPX1 and system Xc⁻, or GCLM, GSS, GPX1 and system Xc⁻ genes. Preferably, the kit comprises combinations of oligonucleotides or polynucleotides such as primer pairs #Hs00157694_ml (Applied Biosystems), and optionally SEQ ID No: 1 able to bind to the transcription product of GCLM gene, or SEQ ID No: 3 and/or 4, and optionally also SEQ ID No: 2 able to bind to the transcription product of the GSS gene, or said kit may comprise SEQ ID No: 6 and/or 7, and optionally SEQ ID No: 5 able to bind to the transcription product of GPX1 gene, or the kit may comprise primer pairs #Hs00374243 (Applied Biosystems), and optionally SEQ ID No: 8 able to bind to the transcription product of system Xc⁻4F2 gene, or it may comprise primer pairs #Hs00204928 (Applied Biosystems), and optionally SEQ ID No: 9 able to bind to the transcription product of Xc⁻xCT gene. The kit most preferably comprises the Applied Biosystems assay #Hs00157694_ml together with SEQ ID No: 3 and SEQ ID No: 4, and optionally SEQ ID No: 1 and SEQ ID No: 2 able to bind to the transcription products of the GCLM and GSS genes. The kit may further comprise a DNA sample collecting means.

In a further aspect of the invention said kit includes a means for determining the level of protein expressed by at least one gene involved in regulating intracellular GSH level. In a preferred embodiment the kit comprises at least one antibody, antibody derivative or antibody fragment able to bind GCL, preferably, GCLM, GSS, GPX, preferably GPX1 and/or system Xc⁻, preferably xCT subunit. In another preferred embodiment, said kit comprises two different antibodies, antibody derivatives or antibody fragments able to bind to GCLM and GSS, or to GCLM and GPX1, or to GSS and GPX1. Most preferably the kit comprises three different antibodies, antibody derivatives or antibody fragments able to bind to GCLM, GSS and GPX1. Alternatively, said kit may comprise antibody, antibody derivative or antibody fragment able to bind able to bind GCLM and system Xc⁻, GSS and system Xc⁻, GPX1 and system Xc⁻, or GCLM, GSS, GPX1 and system Xc⁻.

In yet another aspect the kit for diagnosis of a mental disorder comprises a means for determining the level of activity of the protein expressed by at least one gene involved in regulating intracellular GSH level. Preferably said protein comprises GCL, preferably GCLC, GGT and/or system Xc⁻. According to one embodiment of the invention, the kit comprises means to determine the activity of GCL, preferably GCLC, wherein such means comprise a scintillation analyzer able to determine the amount of ¹⁴C-γ-glutamyl-aminobutyric acid. According to another embodiment of the invention, the kit comprises means to determine the activity of GGT. Preferably such means comprise spectroscopic means able to measure the formation of 5-amino-2-nitrobenzoate. Another embodiment of the invention provides means to determine the activity of system Xc⁻, said means are able to measure [³⁵S] cystine uptake.

Other preferred embodiments provide kits for determining the level of protein or the level of protein activity which further comprise a protein sample collecting means. Most preferably the kits of the invention further comprise a means for collecting a biological sample of a subject, and may in addition also comprise instructions for use of the kit and interpretation of the determined level of expression and/or activity. Preferably, the kits of the invention may be used in the determining step of the methods provided by the invention for measuring the level of expression and/or the level of activity of the at least one gene and/or protein.

In a further aspect of the invention a kit for diagnosis of a mental disorder is provided said kit comprises at least one means for determining the plasmatic level of at least one amino acid, preferably cystine, glutamate, cysteine, homocysteine and/or cysteinyl-glycine. In a preferred embodiment of the invention, said means comprise an amino acid analyzer and or an HPLC. Another aspect of the invention provides a kit which comprises means for determining the GSH levels in blood.

A further aspect of the invention relates to a diagnostic composition or kit for the diagnosis of a mental disorder or a predisposition therefor in a mammal, particularly in a human being, comprising at least one primer or probe for determining the presence of at least one polymorphism of at least one gene involved in regulating the intracellular glutathione level and/or GSH-oxidative stress-related gene expression, wherein said at least one polymorphism is associated with a mental disorder or predisposition therefor.

Preferably, the at least one gene involved in regulating the intracellular GSH level and/or GSH-oxidative stress-related gene expression is selected from a GCLM gene and/or a GSS gene.

The primers and/or probes for determining a single polymorphism in a chromosomal copy of said genes and/or in two chromosomal copies of said genes, the combination of primers and/or probes for determining a combination of polymorphisms in a chromosomal copy of said genes and/or in two chromosomal copies of said genes and/or for determining a combination of polymorphisms in at least one chromosomal copy of a combination of the GCLM and the GSS gene, may be nucleic acid molecules such as DNA and RNA or nucleic acid analogues such as peptide nucleic acids (PNA) or locked nucleic acids (LNA). The primers and/or probes are selected such that they can discriminate between polymorphisms at the position to be analysed. Usually, the primers and/or probes have a length of at least 10, preferably at least 15 up to 50, preferably up to 30 nucleic acid building blocks, e.g. nucleotides. In a preferred embodiment, the composition or kit comprises at least one primer and/or probe and/or at least one combination of primers and/or probes which hybridise to the above-mentioned genes under predetermined conditions, e.g. of temperature, buffer, strength and/or concentration of organic solvent, and which allows a specific determination of the polymorphism to be tested. Preferred examples of such primers are indicated in Table 3 and Table 4 for genotyping with a mass array system.

TABLE 3

| | | | Nucleic acid | SEQ ID |
|---|---|---|---|---|
| Gene | SNP# | PCR primer | sequence | No. |
| GCLM | rs2235971 | PCR primer 1 | ACGTTGGATGCAGATCTGGTAACCACCATC | 20 |
| | | PCR primer 2 | ACGTTGGATGAGTTCTCTGACGCATTTCCG | 21 |
| | rs3170633 | PCR primer 1 | ACGTTGGATGCTTTCTAGATTTTTCACCCAG | 22 |
| | | PCR primer 2 | ACGTTGGATGAGGATGAACTGCTAGCCAAC | 23 |
| | rs2064764 | PCR primer 1 | ACGTTGGATGCCCTCTTCTAGCTTCACTTG | 24 |
| | | PCR primer 2 | ACGTTGGATGAAACACTAGGAACCTTAATC | 25 |
| | rs769211 | PCR primer 1 | ACGTTGGATGGATCATAAGCTTTTGTCTTAC | 26 |
| | | PCR primer 2 | ACGTTGGATGCTGTATTTTTATCACTGTCC | 27 |
| | rs7l8873 | PCR primer 1 | ACGTTGGATGTAACCTCTAGTTGGTTCTGC | 28 |
| | | PCR primer 2 | ACGTTGGATGGGAGTTGAGTGTCATTCCAG | 29 |
| | rs718875 | PCR primer 1 | ACGTTGGATGCTTACCTTCCTGAATTGAGG | 30 |
| | | PCR primer 2 | ACGTTGGATGAATTTCCCTCTGGAAGGATG | 31 |
| | rs2301022 | PCR primer 1 | ACGTTGGATGTGATGCTCAGAGTCACACAC | 32 |
| | | PCR primer 2 | ACGTTGGATGCCTACTGTTATGAAGCACCC | 33 |
| GSS | rs3746450 | PCR primer 1 | ACGTTGGATGCAGGACTTCTCTTTCTCCAG | 34 |
| | | PCR primer 2 | ACGTTGGATGTTATCCTGGGTGACTACCTC | 35 |
| | rs725521 | PCR primer 1 | ACGTTGGATGTAGACCAGTCTCTACAGGTG | 36 |
| | | PCR primer 2 | ACGTTGGATGTCTCATTCCTCCCTGTGATC | 37 |
| | rs1801310 | PCR primer 1 | ACGTTGGATGACGGTTGCAAAGGACTTCTC | 38 |
| | | PCR primer 2 | ACGTTGGATGTTAAATGAGGCCAAGGACCC | 39 |
| | rs2236270 | PCR primer 1 | ACGTTGGATGCCAGTGAGAGCTGATTGTTG | 40 |
| | | PCR primer 2 | ACGTTGGATGGAATCCTCAGGAATCCACAG | 41 |
| | rs2236271 | PCR primer 1 | ACGTTGGATGTTGCGTTTTCACCTTCACCC | 42 |
| | | PCR primer 2 | ACGTTGGATGTTTCCACTGCTTAAAGCAGC | 43 |
| | rs2273684 | PCR primer 1 | ACGTTGGATGTCTGAGAATCAGCTGAGCAC | 44 |
| | | PCR primer 2 | ACGTTGGATGCAGCCCAGCATATTCCAACC | 45 |
| | rs734111 | PCR primer 1 | ACGTTGGATGCTGTGCAATCTTCCAGTTCC | 46 |
| | | PCR primer 2 | ACGTTGGATGCAAACTCTTTCCAGGTAGGG | 47 |
| | rs2025096 | PCR primer 1 | ACGTTGGATGCGAGGTGATGACTGGTATAG | 48 |
| | | PCR primer 2 | ACGTTGGATGTCTTTCTCCAATGAAGAGCC | 49 |
| | rs3761144 | PCR primer 1 | ACGTTGGATGCTTTTGCCTCTAATGCTTTCC | 50 |
| | | PCR primer 2 | ACGTTGGATGAAGTCCCAGAAAAATCCCCC | 51 |

TABLE 4

| | | | SEQ ID |
|---|---|---|---|
| Gene | SNP# | Extension Primer | No |
| GCLM | rs2235971 | CACCATCTTTCCGGCTC | 52 |
| | rs3170633 | CAGTATTTTCAAAATTTGGGAAT | 53 |
| | rs2064764 | CTTTTACTAGTAGGAAAGGAA | 54 |
| | rs769211 | CTTTTGTCTTACAAAAAGGTATTT | 55 |
| | rs7l8873 | TTGGTTCTGCTCCTTCC | 56 |
| | rs718875 | AATTCATCAGGAAAGCCTCA | 57 |
| | rs2301022 | AAACATTGTTCAAAGGACTA | 58 |
| GSS | rs3746450 | GTCCCCCTCCCTCTAGA | 59 |
| | rs725521 | ATCCTTAGCCACCCACT | 60 |
| | rs1801310 | TCATCTGATACCCTGGT | 61 |
| | rs2236270 | TCTGGAAACAGTGTAAATG | 62 |
| | rs2236271 | CCCTGCCATTAAAAATTTTTTCA | 63 |
| | rs2273684 | CTCCCATCACATTCCTG | 64 |
| | rs734111 | GCAGCTCCTGGCCCCCC | 65 |
| | rs2025096 | TTGAACCCATGTCTCTG | 66 |
| | rs3761144 | TAATGCTTTCCCTGCTG | 67 |

Preferred, more preferred and most preferred (a) combinations of polymorphisms, (b) polymorphisms being in linkage disequilibrium with at least one polymorphism of (a), and (c) combinations of polymorphisms of (a) and/or (b), in at least one chromosomal copy of the GCLM gene and/or GSS gene according to the diagnostic composition or kit are the same as mentioned above.

The composition or kit preferably further comprises an enzyme for primer elongation such as a DNA polymerase, nucleotides, e.g. chain elongation nucleotides, such as desoxy nucleoside triphosphates (dNTPs) or chain termination nucleotides such as didesoxynucleoside triphosphates (ddNTPs) (Table 5) and/or labelling groups, e.g. fluorescent or chromogenic labelling groups.

TABLE 5

| Selecting a | |
|---|---|
| termination mix | |
| SNP (bi-allelic) | Termination Mix |
| A/C | CGT (40 Da) |
| A/G | ACT (32 Da) |
| A/T | CGT (25 Da) |
| C/G | ACT (56 Da) or |
| | AGT (24 Da) |
| C/T | ACG (31 Da) |
| G/T | ACT (41 Da) |
| small ins/del | - dependent |
| | on sequence - |

The three nucleotides that are present in the dideoxy-form are shown (e.g. CGT denotes a mixture of dA, ddC, ddG and ddT). Numbers in parentheses are the mass differences between a correct termination and a false termination (i.e. Premature termination caused by pausing of the polymerase)

Still a further aspect of the invention relates to a microarray for the diagnosis of a mental disorder or a predisposition therefor in a mammal, particularly in a human being, comprising a carrier having immobilised thereto at least one probe for determining the presence of at least one polymorphism and/or of at least one combination of polymorphisms of at least one copy of a gene involved in regulating the intracellular GSH level and/or GSH-oxidative stress-related gene expression, wherein said at least one polymorphism is associated with a mental disorder or a predisposition therefor. Preferably, the microarray carrier, e.g. a planar carrier or a microchannel device, has immobilised thereto a plurality of different probes located at different areas on the carrier, which are designed such that they can bind nucleic acid molecules, e.g. RNA molecules or DNA molecules, amplification products, primer elongation products, etc, containing the sequence in which the polymorphism to be tested is located. Thus, an identification of the polymorphism to be analysed by detection of a site-specific binding events of the nucleic acid sample molecule to the probe immobilised on the carrier may be accomplished.

A yet further aspect of the present invention is a primer or probe and/or a combination of primers and/or probes for the diagnosis of a mental disorder or a predisposition therefor in a mammal, particularly in a human being for determining the presence of at least one polymorphism and/or of at least one combination of polymorphisms of at least one copy of a gene involved in regulating the intracellular GSH level and/or GSH-oxidative stress-related gene expression, wherein said at least one polymorphism is associated with a mental disorder or a predisposition therefor. The primers may be nucleic acid molecules such as DNA and RNA or nucleic acid analogues such as peptide nucleic acids (PNA) or locked nucleic acids (LNA). The primer and/or probes are selected such that they can discriminate between polymorphisms at the position to be analysed. Usually, the primers and/or probes have a length of at least 10, preferably at least 15 up to 50, preferably up to 30 nucleic acid building blocks, e.g. nucleotides. In a preferred embodiment, the primer hybridises to the above-mentioned genes under predetermined conditions, e.g. of temperature, buffer, strength and/or concentration of organic solvent, and allows a specific determination of the polymorphism to be tested.

In a further aspect, the present invention is directed to the use of one or more proteins for the manufacture of a medicament for use in the treatment and/or prevention of a mental disorder, wherein the one or more protein is selected from the group consisting of a) GCL, GSS, GPX and system Xc⁻ or a fragment thereof; b) a bioactive protein having a percentage of identity of at least 50% with the amino acid sequence of any one of the proteins of group (a); or c) a bioactive variant of any one of the proteins of group (a) or (b). Preferably GCL comprises GCLM, GPX comprises GPX1, and system Xc⁻ comprises xCT.

The term “protein” as used herein, refers to a polypeptide, peptide, oligopeptide or synthetic oligopeptide. These terms are intended to be used interchangeably. Any one of said terms refers to a chain of two or more amino acids which are linked together with peptide or amide bonds, regardless of post-translational modification such as glycosylation or phosphorylation. The protein may also comprise more than one subunit, where each subunit is encoded by a separate DNA sequence. Amino acid residues are referred to herein by their standard single-letter or three-letter notations: A (Ala) alanine; C (Cys) cysteine; D (Asp) aspartic acid; E (Glu) glutamic acid; F (Phe) phenylalanine; G (Gly) glycine; H (His) histidine; I (Ile) Isoleucine; K (Lys) lysine; L (Leu) leucine; M (Met) methionine; N (Asn) asparagine; P (Pro) proline; Q (Gln) glutamine; R (Arg) arginine; S (Ser) serine; T (Thr) threonine; V (Val) valine; W (Trp) tryptophan; Y (Tyr) tyrosine.

The term “bioactive”, as used herein, refers to a molecule that elicits or affects a biological event. Such biological event may for example be related to a mental disorder such as to schizophrenic disorders, affective disorders, psychoactive substance use disorders, personality disorders, delirium, dementia, epilepsy, panic disorder, obsessive compulsive disorder, intermittent explosive disorder, impulse control disorder, psychosis, attention-deficit-hyperactivity disorder (ADHD), and manic or psychotic depression.

The term “percentage (%) of identity”, or like term, used in respect of the comparison of a reference sequence and another sequence (i.e. a “candidate” sequence), means that in an optimal alignment between the two sequences, the candidate sequence is identical to the reference sequence in a number of subunit positions equivalent to the indicated percentage, the subunits being nucleotides for polynucleotide comparisons or amino acids for protein comparisons. As used herein, an “optimal alignment” of sequences being compared is one that maximizes matches between subunits and minimizes the number of gaps employed in constructing an alignment. Percent identities may be determined with commercially available implementations of algorithms described by Needleman and Wunsch, J. Mol. Biol. 48: 443-453 (1970)(“GAP” program of Wisconsin Sequence Analysis Package, Genetics Computer Group, Madison, Wis.). Other software packages in the art for constructing alignments and calculating percentage identity or other measures of similarity include the “BestFit” program, based on the algorithm of Smith and Waterman, Advances in Applied Mathematics 2: 482-489 (1981) (Wisconsin Sequence Analysis Package, Genetics Computer Group, Madison, Wis.). The percentage of identity may also be generated by WU-BLAST-2 (Altschul et al., Methods in Enzymology 266: 460-480 (1996)). WU-BLAST-2 used several search parameters, most of which are set to the default values. The adjustable parameters are set with the following values: overlap span=1, overlap fraction=0.125, word threshold (T)=11. A % amino acid sequence identity value is determined by the number of matching identical residues divided by the total number of residues in the aligned region. For example, to obtain a protein having an amino acid sequence at least 95% identical to a reference amino acid sequence, up to five percent of the amino acid residues in the reference sequence may be deleted or substituted with another amino acid, or a number of amino acids up to five percent of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence of in one or more contiguous groups with in the references sequence. It is understood that in making comparisons with reference sequences of the invention that candidate sequence may be a component or segment of a larger polypeptide or polynucleotide and that such comparisons for the purpose computing percentage identity is to be carried out with respect to the relevant component or segment.

A protein of the invention also includes a fragment of a protein of the invention. Such protein fragment is meant to be a protein having an amino acid sequence that entirely is the same in part, but not in all, of the amino acid sequence of a protein of the invention. Such protein fragment may be “free-standing,” or may be part of a larger protein of which such protein fragment forms a part or region, most preferably as a single continuous region. Preferably such protein or protein fragment retains the biological activity of the corresponding protein of the invention.

In various other embodiments, the protein (fragment) may be linear or branched, it may comprise modified amino acids, it may be interrupted by non-amino acids, and/or it may be assembled into a complex of more than one polypeptide chain. As is well understood in the art, a protein may be modified naturally or by intervention; for example, disulfide bond formation, glycosylation, lipidation, acetylation, phosphorylation, nitrosylation or any other manipulation or modification, such as conjugation with a labeling component. In some embodiments, protein (fragment) contain one or more analogs of an amino acid (including, for example, unnatural amino acids, etc.), as well as other modifications known in the art. This invention also includes functionally preserved variants of the protein (fragment) described herein. Such variants may be made using methods standard in the art, for example, by conservative amino acid substitutions. Typically such substitutions are among Ala, Val, Leu and Ile; among Ser and Thr; among the acidic residues Asp and Glu; among Asn and Gln; and among the basic residues Lys and Arg; or aromatic residues Phe and Tyr. Particularly preferred are variants in which several, 5 to 10, 1 to 5, or 2 amino acids are substituted, deleted or added, in any combination.

A protein (fragment) of the invention includes isolated naturally occurring proteins. Preferably, such a naturally occurring protein has a frequency in a selected population of at least five percent, and most preferably, of at least ten percent. The selected population may be any recognized population of study in the field of population genetics. Preferably, the selected population is Caucasian, Negroid, or Asian. More preferably, the selected population is French, German, English, Spanish, Swiss, Japanese, Chinese, Korean, Singaporean of Chinese ancestry, Icelandic, North American, Israeli, Arab, Turkish, Greek, Italian, Polish, Pacific Islander, or Indian.

A protein (fragment) of the invention may also include recombinantly produced proteins, synthetically produced proteins and a combination of such proteins of the invention, and fragments thereof. Means for preparing such proteins are well understood in the art. For instance, a protein fragment or a protein of the invention can be isolated from body fluids including, but not limited to, serum, urine, and ascites, or synthesized by chemical or biological methods (for example, cell culture, recombinant gene expression). “Isolated”, if not otherwise specified herein includes the meaning “separated from coexisting material”.

Recombinant proteins of the present invention may be prepared by processes well known in the art from genetically engineered host cells comprising expression systems. Accordingly, the present invention also relates to the production of protein (fragment) by recombinant techniques, to expression systems which comprises a nucleic acid or nucleic acids encoding the proteins of the present invention, to host cells which are genetically engineered with such expression systems, and to methods to isolate the polypeptides.

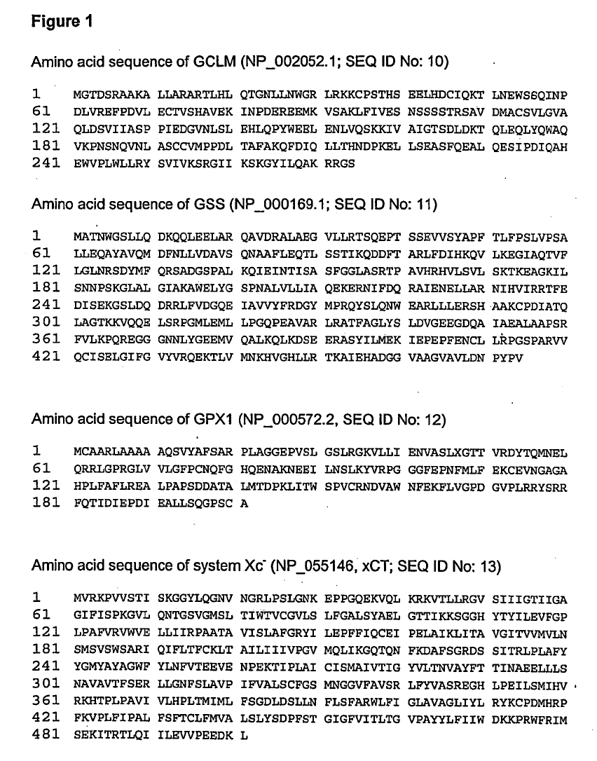

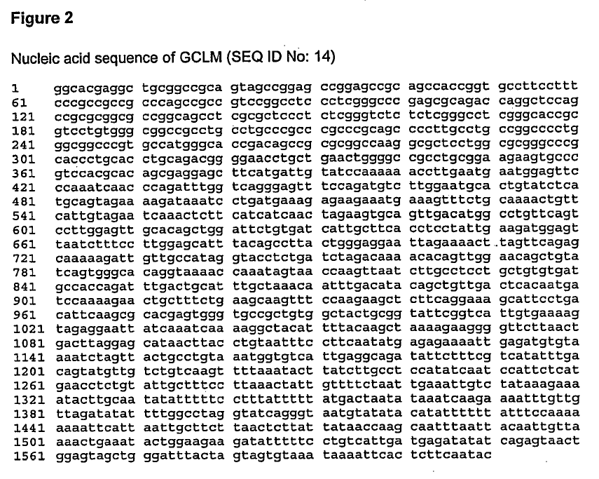

Preferred embodiments provide that the one or more protein of the invention comprises SEQ ID No: 10 (amino acid sequence of GCLM (FIG. 1)), SEQ ID No: 11 (amino acid sequence of GSS (FIG. 1)), SEQ ID No: 12 (amino acid sequence of GPX1 (FIG. 1)) and/or SEQ ID No: 13 (amino acid sequence of system Xc⁻ (xCT) (FIG. 1)).

The protein may also comprise an amino acid sequence having a percentage of identity of at least 50%, preferably at least 60%, more preferred at least 70% or 80%, most preferably at least 90% such as 95%, 97%, or 99% identity with the amino acid sequence of any one of the GCLM, GSS, GPX1 and/or system Xc⁻ (xCT) proteins.

Another aspect of the invention encompasses the use of one or more polynucleotide for the manufacture of a medicament for use in the treatment and/or prevention of a mental disorder. According to the invention the one or more polynucleotide comprises a sequence encoding a protein of the invention as defined further above and said sequence being operatively associated with a tissue specific or a constitutive promoter.

The term “polynucleotides” means natural or semi-synthetic or synthetic or modified nucleic acid molecules. It refers to nucleotide sequences or oligonucleotides including deoxyribonucleic acid (DNA) and/or ribonucleic acid (RNA) and/or modified nucleotides. These terms are intended to be used interchangeably. RNA may be in the form of an tRNA (transfer RNA), snRNA (small nuclear RNA), rRNA (ribosomal RNA), mRNA (messenger RNA), anti-sense RNA, and ribozymes. DNA may be in form of plasmid DNA, viral DNA, linear DNA, chromosomal or genomic DNA, cDNA, or derivatives of these groups. In addition these DNAs and RNAs may be single, double, triple, or quadruple stranded. The term also includes PNAs (peptide nucleic acids), phosphorothioates, and other variants of the phosphate backbone of native nucleic acids.

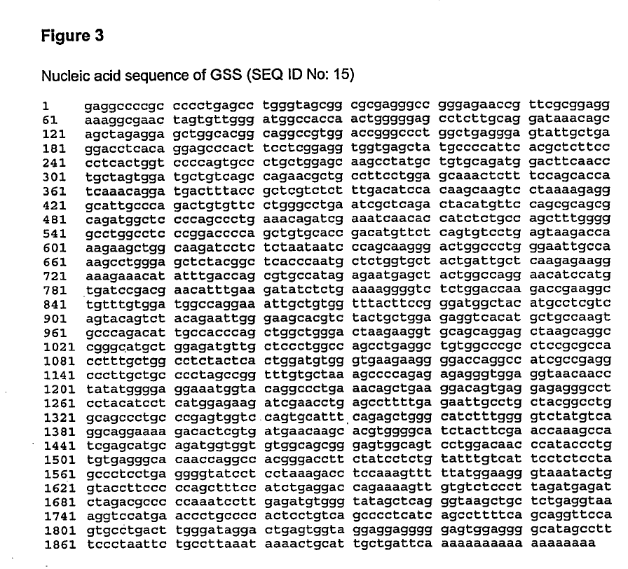

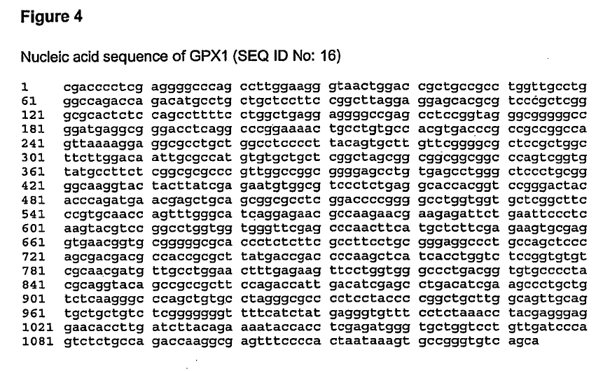

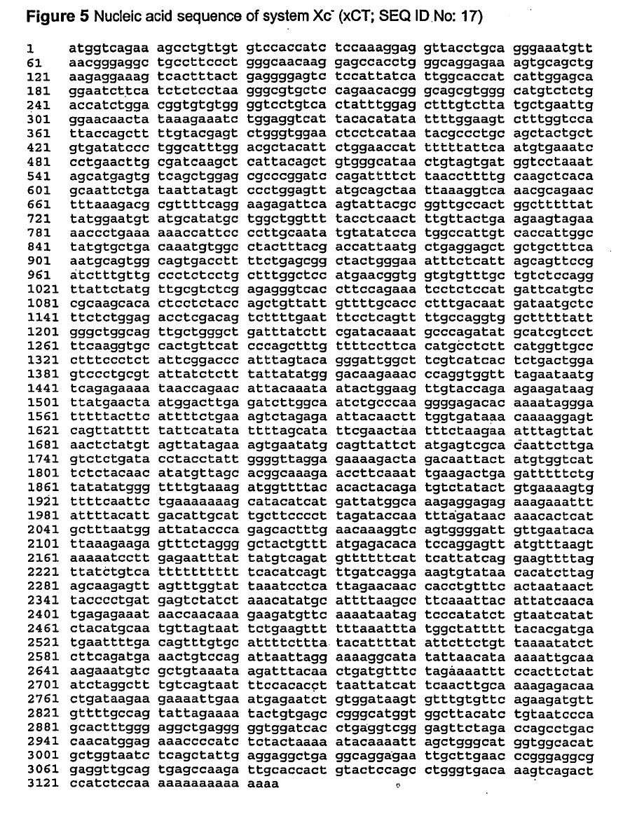

Preferably the one or more polynucleotide of the invention comprises SEQ ID No: 14 (nucleic acid sequence of GCLM (FIG. 2)), SEQ ID No: 15 (nucleic acid sequence of GSS (FIG. 3)), SEQ ID No: 16 (nucleic acid sequence of GPX1 (FIG. 4)) and/or SEQ ID No: 17 (nucleic acid sequence of system Xc⁻ (xCT) (FIG. 5)).

Other embodiments of the invention provide polynucleotides which hybridize under stringent conditions to any one of SEQ ID No: 14 to SEQ ID No: 19. “Stringent conditions” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends upon the ability of a denatured nucleic acid to reanneal when complementary strands are present in an environment near but below their melting temperature. The higher the degree of homology between the probe and the hybridizable sequence such as SEQ ID No: 14 to 19, the higher the relative temperature which can be used. As a result, it follows that higher relative temperatures would tend to make the reaction conditions more stringent, while lower temperatures less so. Moreover, stringency is also inversely proportional to salt concentrations. “Stringent conditions” are exemplified by reaction conditions characterized by: (1) low ionic strength and high temperature for washing, for example 0.015 M sodium chloride/0.0015 M sodium citrate/0.1% sodium dodecyl sulfate at 50° C.; (2) the use of a denaturing agent, such as formamide, for example, 50% (vol/vol) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C. Alternatively, stringent conditions can be: 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. For additional details and explanation of stringency of hybridization reactions, see Ausubel et al., Protocols in Molecular Biology (1995).

In a preferred embodiment, the polynucleotide is included in a viral vector, wherein the polynucleotide is operably linked to a promoter of the viral genome. The promoter sequences can be discerned by searching the publicly available databases. Preferably the viral vectors are able to selectively replicate in a specific tissue such as brain cells of a subject affected or at risk of being affected by the mental disorder, but not in a non-diseased cell. The replication is conditional upon the presence in a diseased cell, and not in a non-diseased (=standard expression profile) cell, of positive transcription factors that activate the promoter of the disclosed genes involved in regulating intracellular GSH level. It can also occur by the absence of transcription inhibiting factors that normally occur in a non-diseased cell and prevent transcription as a result of the promoter. Accordingly, when transcription occurs, it proceeds into the gene essential for replication, such that in the diseased cell, but not in non-diseased cell, replication of the vector and its attendant functions occur. With this vector, a diseased cell, e.g., a brain cell, can be selectively treated, with minimal systemic toxicity.

In one embodiment, the viral vector is an adenoviral vector, which includes a coding region of a gene essential for replication of the vector, wherein the coding region is selected from the group consisting of E1a, E1b, E2 and E4 coding regions. The term “gene essential for replication” refers to a nucleic acid sequence whose transcription is required for the vector to replicate in the target cell. Preferably, the gene essential for replication is selected from the group consisting of the E1A and E1b coding sequences. Particularly preferred is the adenoviral E1A gene as the gene essential for replication. Methods for making such vectors are well know to the person of ordinary skill in the art as described, e.g., in Sambrook et al., in Molecular Cloning: A Laboratory Manual, Cold Spring Harbor, N.Y., 1989.

The vector of the present invention can be transfected into a helper cell line for viral replication and to generate infectious viral particles. Alternatively, transfection of the vector or other nucleic acid constructs harboring a polynucleotide of the invention as described above into e.g. a nerve cell can take place by electroporation, calcium phosphate precipitation, microinjection, or through liposomes, including proteoliposomes.

Further aspects of the present invention provide methods for prevention and/or treatment of a mental disorder such as schizophrenia comprising administering an effective amount of one or more proteins or of one or more polynucleotides to a mammal including a human. Preferably the one or more protein is selected from the group consisting of a) GCL, GSS, GPX and/or system Xc⁻ or a fragment thereof; b) a bioactive protein having a percentage of identity of at least 50% with the amino acid sequence of any one of the proteins of group (a); or c) a bioactive variant of any one of the proteins of group (a) or (b). Preferably GCL comprises GCLM, GPX comprises GPX1, and system Xc⁻ comprises xCT. Most preferably said one or more protein comprises SEQ ID No: 10, SEQ ID No: 11, SEQ ID No: 12 and/or SEQ ID No: 13. The one or more polynucleotide preferably comprises a sequence encoding a protein as defined above and which is operatively associated with a tissue specific or a constitutive promoter. Said one or more polynucleotide most preferably comprises SEQ ID No: 14, SEQ ID No: 15, SEQ ID No: 16 and/or SEQ ID No: 17.

In a further aspect, the invention is directed to a method for prevention and/or treatment of a mental disorder which includes administering an effective amount of an agent that can alter the expression of at least one gene or an agent that can alter the activity of at least one protein involved in regulating intracellular GSH level. Preferably the agent can alter the expression of the GCL, preferably of GCLM, GSS, GPX, preferably of GPX1 and/or system Xc⁻, preferably of xCT, gene and/or the activity GCL, preferably of GCLC, GGT and/or system Xc⁻. A preferred embodiment provides that the agent which alters the activity of GCLC is GCLM, functional homologue, derivative a fragment thereof.

According to another aspect, the invention provides a method for prevention and/or treatment of a mental disorder comprising administering an effective amount of an agent that can normalize GSH levels, preferably intracellular GSH level. Most preferably the intracellular GSH level of blood cells is determined. Said agent may comprise GSH, a precursor, derivative and or chemical equivalent thereof. Preferably such agent is N-acetyl cysteine (NAC). Most preferably said agent is R(−)-2-oxothiazolidine-4-carboxylic acid (OTC).

“Mammal” for purposes of prevention and/or treatment refers to any animal classified as a mammal, including humans, domestic and from animals, and zoo, sports, or pet animals, such as dogs, horses, cats, sheep, pigs, cattle, etc. Preferably, the mammal is human.

The term “treatment” as used herein refers to both therapeutic treatment and prophylactic or preventative measures. Accordingly said methods of the invention include both prophylactic and therapeutic methods of treating a subject affected, or at risk of being affected by a mental disorder such as schizophrenia and in which the disorder is to be prevented. Subjects at risk for such disorders can be identified by diagnostic methods and kits e.g., as described above. Administration of the polynucleotide, protein or agent that can alter the expression or activity of the protein according to the invention may occur prior to the manifestation of symptoms characteristic for a mental disorder, such that development of e.g. schizophrenia is prevented or delayed in its progression.

A “effective amount” of a polynucleotide, a protein or an agent that can alter the expression of the gene or activity of the protein refers to a sufficient amount of one of these therapeutic agents to effect beneficial or desired results including preventing the onset of a mental disorder such as schizophrenia, decreasing one or more symptoms resulting from the disorder, increasing the quality of life of those affected by the disorder, decreasing the dose of other medications required to treat the disorder, enhancing the effect of another medication and/or delaying the progression of the disorder.

The determination of an effective amount is well within the capability of those skilled in the art. For any therapeutic, the effective dose can be estimated initially either in cell culture assays, e.g., of neoplastic cells, or in animal models, usually mice, rabbits, dogs, or pigs. The animal model may also be used to determine the appropriate concentration range and route of administration. Such information can then be used to determine useful doses and routes for administration in humans.

Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between toxic and therapeutic effects is the therapeutic index, and it can be expressed as the ratio, LD50/ED50. The data obtained from cell culture assays and animal studies is used in formulating a range of dosage for human use. The dosage contained in such compositions is preferably within a range of circulating concentrations that include the ED50 with little or no toxicity. The dosage varies within this range, depending upon the dosage form employed, sensitivity of the patient, and the route of administration.

The exact dosage will be determined by the practitioner, in light of factors related to the subject that requires treatment. Dosage and administration are adjusted to provide sufficient levels of the active moiety or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy.

Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for polynucleotide, protein or agents.

An effective amount can be administered in one or more administrations and may or may not be achieved in conjunction with another drug, compound, or pharmaceutical effective amount if, in conjunction with one or more other agents, a desirable result may be or is achieved.

For therapeutic applications, the protein or polynucleotide of the invention (whether entrapped in a liposome or contained in a viral vector) or the agents as described above are preferably administered as pharmaceutical compositions containing the therapeutic agent in combination with one or more pharmaceutically acceptable carriers.

A further aspect of the invention encompasses a pharmaceutical composition comprising one or more active ingredients which increase the intracellular GSH level and, optionally, a pharmaceutically acceptable carrier, diluent and/or adjuvant for use in the treatment and/or prevention of a mental disorder in patients having at least one polymorphism and/or at least one combination of polymorphisms of at least one copy of a gene involved in regulating the intracellular GSH level and/or GSH-oxidative stress-related gene expression.

According to the invention, a pharmaceutical composition preferably contains the therapeutic agent in combination with one or more pharmaceutically acceptable carriers.

Preferably, the active ingredient of the pharmaceutical composition is a protein selected from the group consisting of (a) GCLM and/or GSS or a fragment thereof, (b) a bioactive protein having a percentage of identity of at least 50% with the amino acid sequence of any one of the proteins of a), and (c) a bioactive variant of any one of the proteins a) or b). The terms “protein”, “bioactive”, “fragment”, “variant” or “percentage (%) of identity” shall have the meaning as defined above.

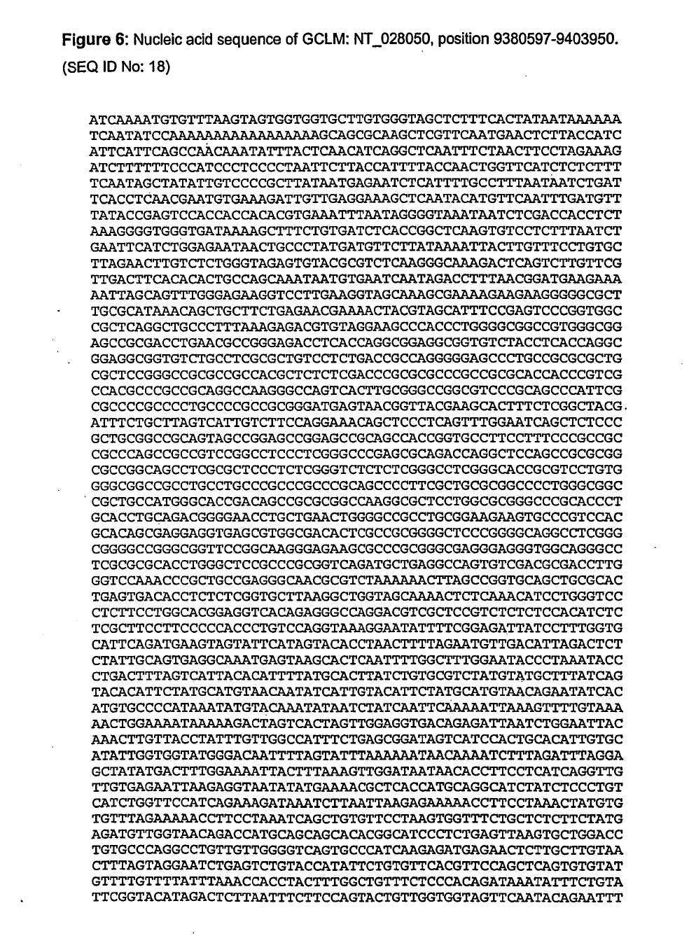

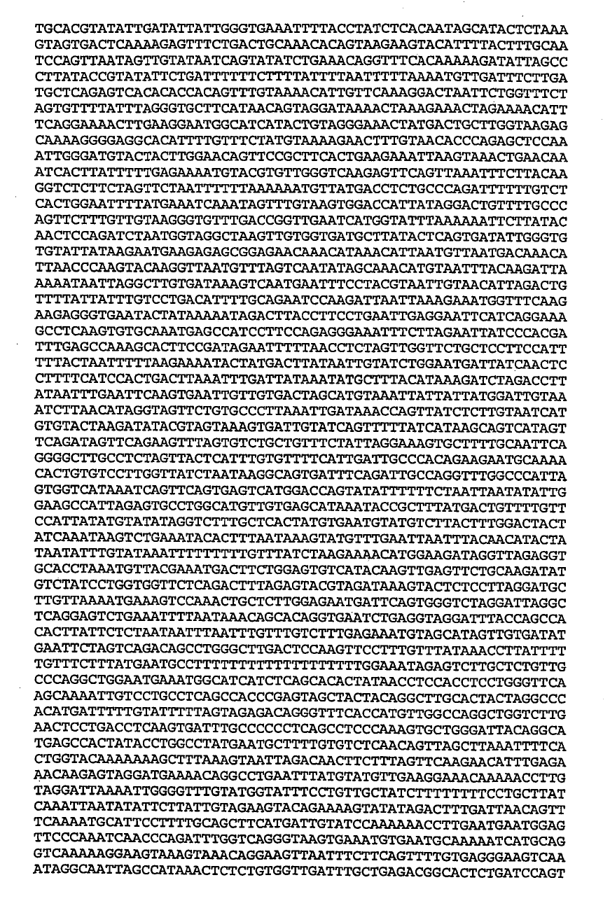

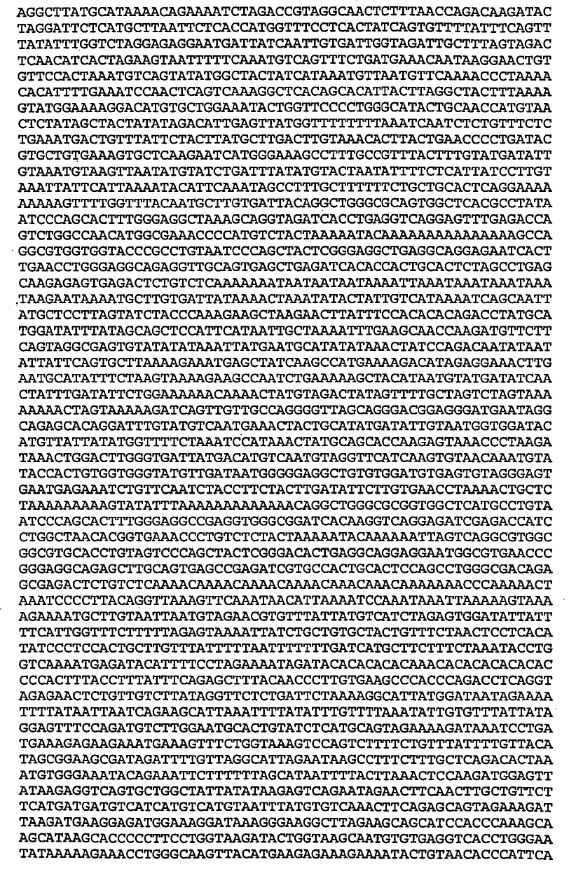

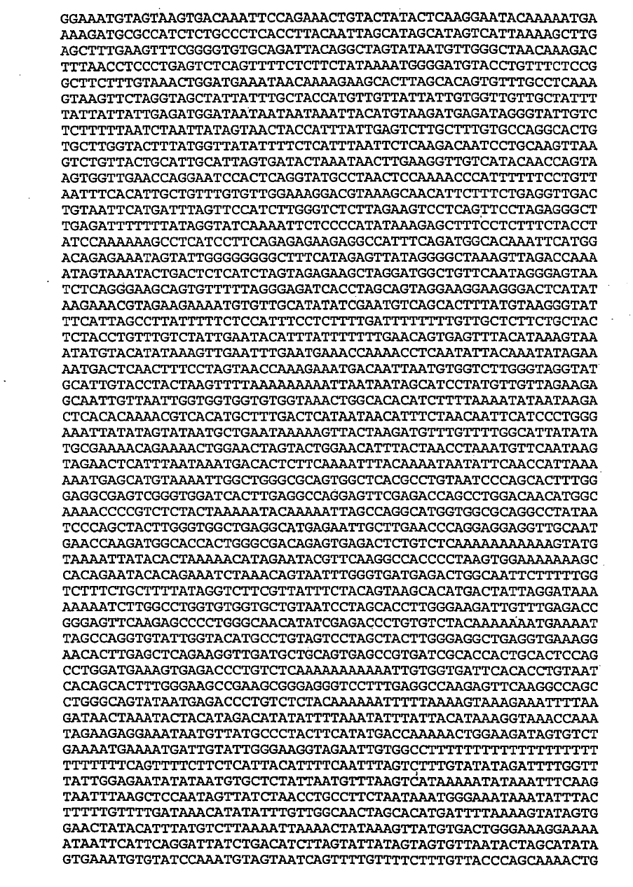

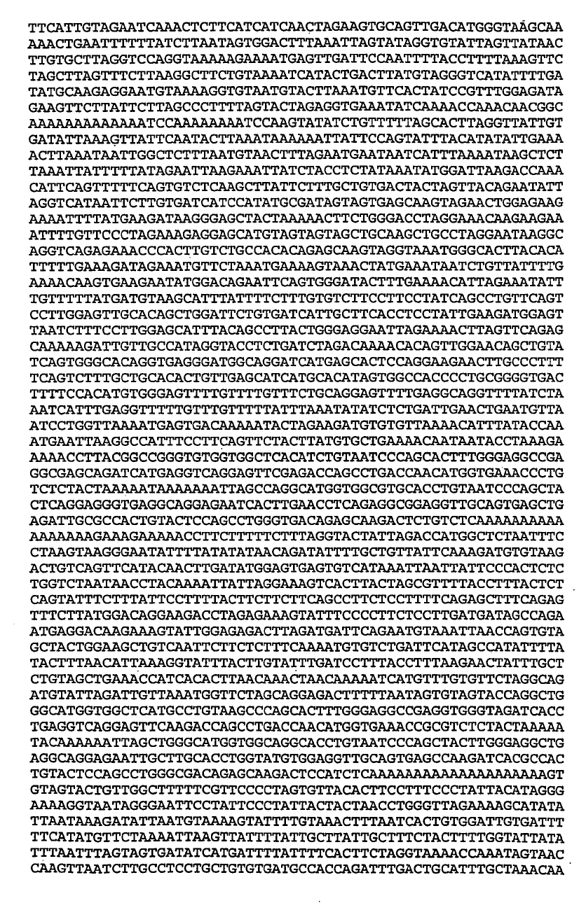

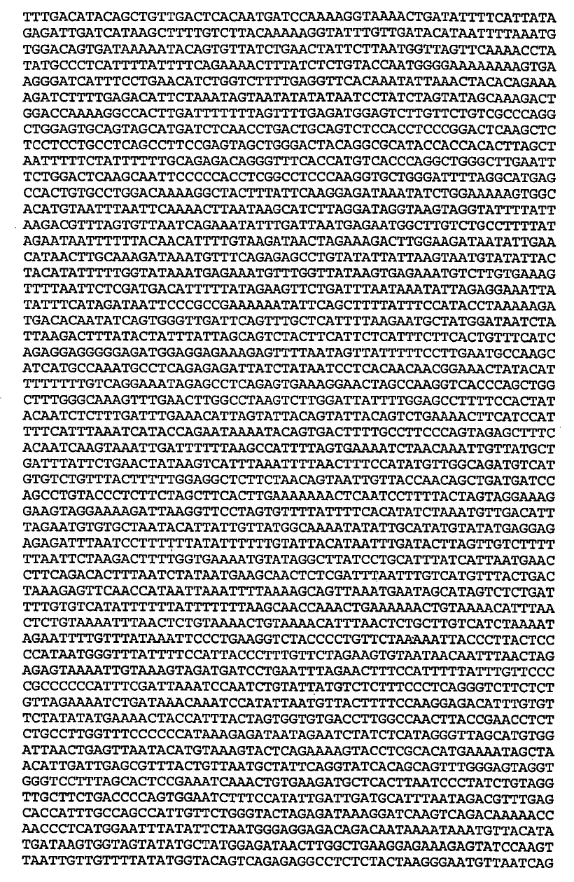

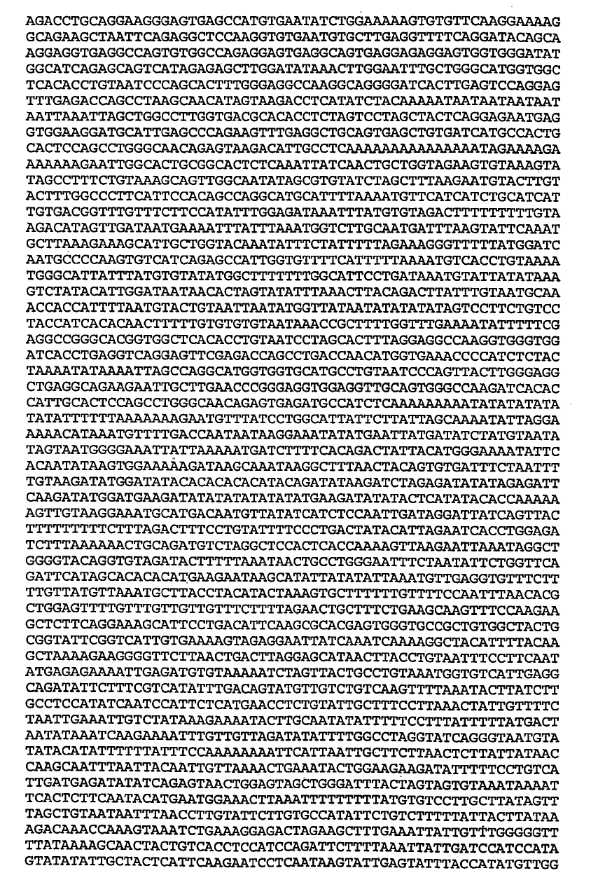

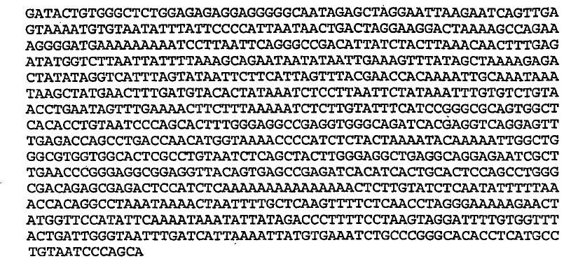

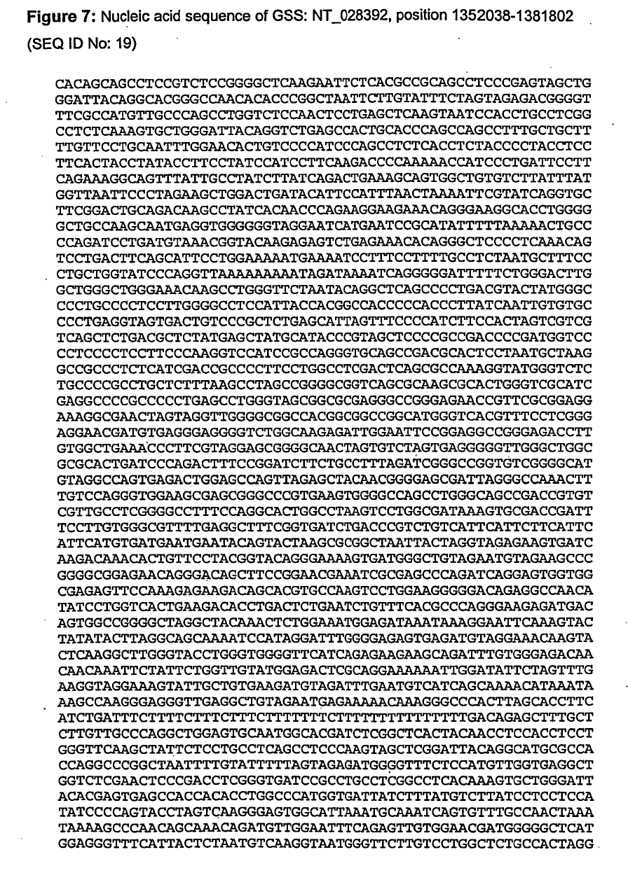

Preferred embodiments provide that the one or more protein of the invention comprises SEQ ID No: 10 (amino acid sequence of GCLM (FIG. 1)) and SEQ ID No: 11 (amino acid sequence of GSS (FIG. 1)). The protein may also comprise an amino acid sequence having a percentage of identity of at least 50%, preferably at least 60%, more preferred at least 70% or 80%, most preferably at least 90% such as 95%, 97%, or 99% identity with the amino acid sequence of any one of the GCLM and/or GSS proteins. A still further preferred aspect of the invention relates to a pharmaceutical composition wherein the active ingredient is a polynucleotide comprising a sequence encoding a protein as defined above. Preferably the one or more polynucleotide of the invention comprises SEQ ID No: 18 (nucleic acid sequence of GCLM (FIG. 6)) and SEQ ID No: 19 (nucleic acid sequence of GSS (FIG. 7)). Other embodiments of the invention provide polynucleotides which (nucleic acid sequence of GCLM (FIG. 6)) and SEQ ID No: 19 (nucleic acid sequence of GSS (FIG. 7)). Other embodiments of the invention provide polynucleotides which hybridize under stringent conditions to SEQ ID Nos: 18 and 19. In a preferred embodiment, the polynucleotide is included in a viral vector, wherein the polynucleotide is operably linked to a promoter of the viral genome. The terms “polynucleotides”, “stringent conditions” or “viral vector” shall have the meaning as defined above.

In another preferred embodiment of the invention, the active ingredient of the pharmaceutical composition is GSH or a compound increasing the GSH level, preferably the intracellular GSH level. Said compound may comprise GSH, a precursor, derivative and/or chemical equivalent thereof. Preferably, such an agent is N-acetyl cysteine (NAC). Most preferably, said agent is R(−)-2-oxothiazolidine-4-carboxylic acid (OTC). In a preferred embodiment, the patients have at least one polymorphism, at least one genotype and/or at least one combination of polymorphisms as specifically indicated above.

The compositions according to the invention may be administered alone or in combination with at least one other agent, such as stabilizing compound, which may be administered in any sterile, biocompatible pharmaceutical carrier, including, but not limited to, saline, buffered saline, dextrose and water. The compositions may be administered to a patient alone or in combination with other agents, drugs or hormones. Said patient may have one or a plurality of polymorphisms and/or at least one combination of polymorphisms of at least one genomic copy.

The pharmaceutical compositions may be administered by any number of routes, including, but not limited to, oral, sublingual, intravenous, intramuscular, intraarticular, intraarterial, intramedullary, intrathecal, intraventricular, intraoccular, intrathecal, intracereberal, intracranial, respiratoral, intratracheal, nasopharyngeal, transdermal, intradermal, subcutaneous, intraperitoneal, intranasal, enteral, topical, or via rectal means, infusion or implant. Preferably, said route of administration is oral.

The pharmaceutical composition may be used in the methods of treatment of the present invention. Such compositions are preferably sterile and contain an effective amount of a protein or a polynucleotide for inducing the desired response in a unit of weight or volume suitable for administration to a patient.

When administered, the pharmaceutical composition of the present invention is administered in pharmaceutically acceptable preparations. The term “pharmaceutically-acceptable carrier” as used herein means one or more compatible solid or liquid fillers, diluents or encapsulating substances which are suitable for administration into mammals including humans. The term “carrier” denotes an organic or inorganic ingredient, natural or synthetic, with which the active ingredient is combined to facilitate the application.

The term “pharmaceutically acceptable” means a non-toxic material that does not interfere with the effectiveness of the biological activity of the active ingredients. Such preparations may routinely contain pharmaceutically acceptable concentrations of salts, buffering agents, preservatives, compatible carriers, supplementary immune potentiating agents such as adjuvants and cytokines and optionally other therapeutic agents, such as chemotherapeutic agents.

When used in medicine, the salts should be pharmaceutically acceptable, but non-pharmaceutically acceptable salts may conveniently be used to prepare pharmaceutically-acceptable salts thereof and are not excluded from the scope of the invention.

The pharmaceutical compositions may contain suitable buffering agents, including: acetic acid in a salt; citric acid in a salt; boric acid in a salt; and phosphoric acid in a salt.

The pharmaceutical compositions also may contain, optionally, suitable preservatives, such as: benzalkonium chloride; chlorobutanol; parabens and thimerosal.

The doses of protein, polynucleotide or pharmaceutical composition administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as a syrup, elixir or an emulsion.

Compositions suitable for parenteral administration conveniently comprise a sterile aqueous or non-aqueous preparation of a polypeptide or nucleic acid encoding the polypeptide, which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables.

Carrier formulation suitable for oral, subcutaneous, intravenous, intramuscular, etc. administrations can be found in Remington's Pharmaceutical Sciences, Mack Publishing Co., Easton, Pa.

Another aspect of the invention encompasses the use of one or more active ingredients as defined above for the manufacture of a medicament which increases the intracellular GSH level for use in the treatment and/or prevention of a mental disorder in patients having at least one polymorphism and/or at least one combination of polymorphisms of at least one copy of a gene involved in regulating the intracellular glutathione (GSH) level and/or GSH-oxidative stress-related gene expression.

In a still further aspect of the invention, the active ingredient is a protein selected from the group consisting of (a) GCLM and/or GSS or a fragment thereof, (b) a bioactive protein having a percentage of identity of at least 50% with the amino acid sequence of any one of the proteins of a), and (c) a bioactive variant of any one of the proteins of (a) or (b). The terms “protein”, “bioactive”, “fragment”, “variant” or “percentage (%) of identity” shall have the meaning as defined above.

In another aspect of the invention, the active ingredient is a polynucleotide comprising a sequence encoding a protein as defined above. In a further aspect, the active ingredient is GSH or a compound increasing the intracellular GSH level. In a preferred embodiment, the patients have at least one polymorphism, at least one genotype, and/or at least one combination of polymorphisms as specifically indicated above.

A further aspect of the invention relates to the use of a compound effective against mental disorders for the manufacture of a medicament for administration to patients having at least one polymorphism and/or at least one combination of polymorphisms of at least one copy of a gene involved in regulating the intracellular glutathione (GSH) level.

Further aspects of the present invention provide a method of preventing and/or treating a mental disorder such as schizophrenia, comprising administering a medicament which is effective against mental disorders and/or increases the intracellular GSH level, to a patient having at least one polymosphism and/or at least one combination of polymorphisms of at least one copy of a gene involved in regulating the intracellular glutathione (GSH) level, and/or GSH-oxidative stress-related gene expression. Preferably, the medicament comprises at least one compound selected from the group consisting of (a) anti-epileptic drugs selected from barbiturates and derivatives thereof, benzodiazepines, carboxamides, hydantoins, succinimides, valproic acid and other fatty acid derivates and other anti-eplileptic drugs, (b) conventional antipsychotics and (c) atypical antipsychotics, in which the active ingredients are present in each case in free form or in the form of a pharmaceutically acceptable salt and optionally at least one pharmaceutically acceptable carrier; for simultaneous, separate or sequential use.

The term “barbiturates and derivatives thereof” as used herein includes, but is not limited to phenobarbital, pentobarbital, mepobarbital and primidon. The term “benzodiazepines” as used herein includes, but is not limited to clonazepam, diazepam and lorazepam. The term “carboxamides” as used herein includes, but is not limited to carbamazepine, oxcarbazepine, 10-hydroxy-10,11-dihydrocarbamazepine and the compounds of formula II

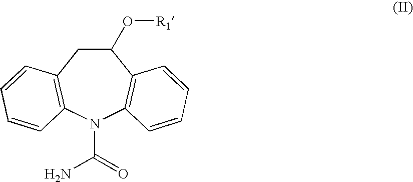

wherein R₁′ represents C₁-C₃alkyl carbonyl. The term “hydantoins” as used herein includes, but is not limited to phenyloin. The term “succinimides” as used herein includes, but is not limited to ethosuximide, phensuximide and mesuximide. The term “valproic acid and other fatty acid derivates” as used herein includes, but is not limited to valproic acid sodium salt, tiagabine hydrochloride monohydrate and vigrabatrine. The term “other anti-epileptic drugs” as used herein includes, but is not limited to levetiracetam, lamotrigine, gabapentin, sultiam, felbamate, the 1,2,3-1H-triazoles disclosed in EP 114 347 and the 2-aryl-8-oxodihydropurines disclosed in WO99/28320.

The term “conventional antipsychotics” as used herein includes, but is not limited to haloperidol, fluphenazine, thiotixene and flupentixol.

The term “atypical antipsychotics” as used herein relates to clozaril, risperidone, olanzapine, quetiapine, ziprasidone and aripiprazol.

The structure of the active ingredients identified by code nos., generic or trade names may be taken from the actual edition of the standard compendium “The Merck Index” or from databases, e.g. Patents International (e.g. IMS World Publications). The corresponding content thereof is hereby incorporated by reference. Any person skilled in the art is fully enabled to identify the active ingredients and, based on these references, likewise enabled to manufacture and test the pharmaceutical indications and properties in standard test models, both in vitro and in vivo.

Phenobarbital, can be administered, e.g., in the form as marketed, e.g. under the trademark Luminal™. Primidon can be administered, e.g., in the form as marketed, e.g. under the trademark Mylepsinum™. Clonazepam can be administered, e.g., in the form as marketed, e.g. under the trademark Antelepsin™. Diazepam can be administered, e.g., in the form as marketed, e.g. under the trademark Diazepam Desitin™. Lorazepam can be administered, e.g., in the form as marketed, e.g. under the trademark Tavor™. Carbamazepine can be administered, e.g., in the form as marketed, e.g. under the trademark Tegretal™ or Tegretol™. Oxcarbazepine can be administered, e.g., in the form as marketed, e.g. under the trademark Trileptal™. Oxcarbazepine is well known from the literature [see for example Schuetz H. et al., Xenobiotica (GB), 16(8), 769-778 (1986)]. The preparation of the compound of formula II wherein R₁′ is C₁-C₃alkyl carbonyl and its pharmaceutically acceptable salts is described, e.g., in U.S. Pat. No. 5,753,646. 10-Hydroxy-10,11-dihydrocarbamazepine can be prepared as disclosed in U.S. Pat. No. 3,637,661. 10-Hydroxy-10,11-dihydrocarbamazepine may be administered, e.g., in the form as described in U.S. Pat. No. 6,316,417. Phenyloin can be administered, e.g., in the form as marketed, e.g. under the trademark Epanutin™. Ethosuximide can be administered, e.g., in the form as marketed, e.g. under the trademark Suxinutin™. Mesuximide can be administered, e.g., in the form as marketed, e.g. under the trademark Petinutin™. Valproic acid sodium salt can be administered, e.g., in the form as marketed, e.g. under the trademark Leptilan™. Tiagabine hydrochloride monohydrate can be administered, e.g., in the form as marketed, e.g. under the trademark Gabitril™. Vigabatrine can be administered, e.g., in the form as marketed, e.g. under the trademark Sabril™. Levetiracetam can be administered, e.g., in the form as marketed, e.g. under the trademark Keppra™. Lamotrigine can be administered, e.g., in the form as marketed, e.g. under the trademark Lamictal™. Gabapentin can be administered, e.g., in the form as marketed, e.g. under the trademark Neurontin™. Sultiam can be administered, e.g., in the form as marketed, e.g. under the trademark Ospolot™. Felbamate can be administered, e.g., in the form as marketed, e.g. under the trademark Taloxa™. Topiramate can be administered, e.g., in the form as marketed, e.g. under the trademark Topamax™. The 1,2,3-1H-triazoles disclosed in EP 114 347 may be administered, e.g., in the form as described in U.S. Pat. No. 6,455,556. The 2-aryl-8-oxodihydropurines disclosed in WO99/28320 may be administered, e.g., in the form as described in WO99/28320. Haloperidol can be administered, e.g., in the form as marketed, e.g. under the trademark Haloperidol STADA™. Fluphenazine can be administered, e.g., in the form of its dihydrochloride as marketed, e.g. under the trademark Prolixin™. Thiothixene can be administered, e.g., in the form as marketed, e.g. under the trademark Navane™. It can be prepared, e.g., as described in U.S. Pat. No. 3,310,553. Flupentixol can be administered for instance in the form of its dihydrochloride, e.g., in the form as marketed, e.g. under the trademark Emergil™ or in the form of its decanoate, e.g., in the form as marketed, e.g. under the trademark Depixol™. It can be prepared, e.g., as described in BP 925,538. Clozaril can be administered, e.g., in the form as marketed, e.g. under the trademark Leponex™. It can be prepared, e.g., as described in U.S. Pat. No. 3,539,573. Risperidone can be administered, e.g., in the form as marketed, e.g. under the trademark Risperdal™. Olanzapine can be administered, e.g., in the form as marketed, e.g. under the trademark Zyprexa™. Quetiapine can be administered, e.g., in the form as marketed, e.g. under the trademark Seroquel™. Ziprasidone can be administered, e.g., in the form as marketed, e.g. under the trademark Geodon™. It can be prepared, e.g., as described in GB 281,309. Aripiprazole can be administered, e.g., in the form as marketed, e.g. under the trademark Abilify™. It can be prepared, e.g., as described in U.S. Pat. No. 5,006,528. Topiramate can be administered, e.g., in the form as marketed, e.g. under the trademark Topamax™.

Phenobarbital may be administered to an adult patient in a total daily dosage between about 1 to about 3 mg/kg body weight and to a pediatric patient in a total daily dosage between about 3 to about 4 mg/kg body weight, split into two separate units. Primidone may be administered to an adult patient and to children being at least 9 years old in a total daily dosage of 0.75 to 1.5 g. Clonazepam may be administered to an adult patient in a total daily dosage between about 3 to about 8 mg and to a pediatric patient in a total daily dosage between about 0.5 to about 3 mg, split into three of four separate units. Diazepam may be administered to an adult patient in a total daily dosage between about 5 to about 10 mg and to a pediatric patient in a total daily dosage between about 5 to about 10 mg. Lorazepam may be administered to an adult patient in a total daily dosage between about 0.044 mg/kg body weight to about 0.05 mg/kg body weight. Carbamazepine may be administered to an adult patient in a total daily dosage between about 600 to about 2000 mg and to a pediatric patient older than 6 years in a total daily dosage between about 400 to about 600 mg. Oxcarbazepine may be administered to an adult patient in a total daily dosage between about 600 to about 2400 mg and to a pediatric patient in a total daily dosage between about 30 to about 46 mg/kg body weight. Phenyloin may be administered to an adult patient in a total daily dosage between about 100 to about 300 mg and to a pediatric patient in a total daily dosage between about 100 to about 200 mg. Ethosuximide may be administered to an adult patient in a total daily dosage of about 15 mg/kg body weight and to a pediatric patient in a total daily dosage of about 20 mg/kg body weight. Valproic acid sodium salt may be administered to an adult patient in a total daily dosage of about 20 mg/kg body weight and to a pediatric patient in a total daily dosage of about 30 mg/kg body weight. Tiagabine hydrochloride monohydrate may be administered to an adult patient in a total daily dosage between about 15 to about 70 mg. Vigrabatrine may be administered to an adult patient in a total daily dosage between about 2 to about 3 g. Levetiracetam may be administered to patient who is older than 16 years in a total daily dosage between about 1000 to about 3000 mg. Lamotrigine may be administered to patient who is older than 12 years in a total daily dosage between about 100 to about 200 mg. Gabapentin may be administered to patient in a total daily dosage between about 900 to about 2400 mg. Sultiam may be administered to patient in a total daily dosage between about 5 to about 10 mg/kg body weight. Felbamate may be administered to patient who is older than 14 years in a total daily dosage of between about 2400 to about 3600 mg. Topiramate may be administered to an adult patient in a total daily dosage of between about 250 to about 500 mg. Clozaril may be administered to an adult patient in a total daily dosage of between about 300 to about 900 mg. Haloperidol may be administered to a patient in a total daily dosage of between about 2.5 to about 30 mg. Olanzapine can be administered to a patient in a total daily dosage of between about 2.5 to about 20 mg. Quetiapine can be administered to a patient in a total daily dosage of between about 500 to about 600 mg. Risperidone may be administered to a patient in a total daily dosage of between about 2 to about 6 mg.

In yet other aspects the invention relates to methods for screening for a modulator of a mental disorder. Such methods include in vivo or cell-based and/or cell-free assays to identify compounds which are capable of interfering with the expression of at least one gene involved in regulating intracellular GSH and/or which are capable of interacting with a protein involved in regulating intracellular GSH or protein-binding partner to alter the activity of the protein or its binding partner. Such assays can also be used to identify compounds which modulate the interaction between the protein and its binding partner such as a target peptide. According to one aspect of the invention, such method comprises the steps (a) determining the level of expression of at least one gene involved in regulating intracellular GSH in a sample of cells; (b) contacting the sample of cells with a candidate agent; (c) determining the level of expression of the at least one gene of step (a) for the sample of cells of step (b); and (d) comparing the levels of expression determined in step (a) and (c), wherein an alteration in the level of expression of the at least one gene indicates that the candidate agent is a modulator of the mental disorder.

According to another aspect, such method for screening for a modulator of a mental disorder comprises steps (a) administering a candidate agent to a non-human test animal which is predisposed to be affected or which is affected by a mental disease; (b) administering the candidate agent of step (a) to a matched control non-human animal not predisposed to be affected or at risk of being affected by a mental disease; (c) determining the level of expression of at least one gene involved in regulating intracellular GSH in vivo or in vitro in a biological sample isolated from the animal of step (a) and (b); and (d) comparing the levels of expression of step (c); wherein an alteration in the level of expression of the at least one gene indicates that the candidate agent is a modulator of the mental disorder. Preferably the at least one gene involved in regulating the intracellular GSH level comprise GCL, preferably GCLM, GSS, GPX preferably GPX1 and/or system Xc⁻ gene.

According to a still further aspect such method for screening for a modulator of a mental disorder includes step (a) determining the level of activity of at least one protein involved in regulating intracellular GSH in a sample of cells; (b) contacting said sample of cells with a candidate agent; and (c) determining the level of activity of the at least one protein of step (a) for the sample of cells of step (b); and (d) comparing the activity determined in step (a) and (c), wherein an alteration in the activity of the at least one protein indicates that the candidate agent is a modulator of the mental disorder. In yet another aspect a method for screening for a modulator of a mental disorder comprises the following steps: (a) administering a candidate agent to a non-human test animal which is predisposed to be affected or which is affected by a mental disease; (b) administering the candidate agent of step (a) to a matched control non-human animal not predisposed to be affected or being affected by a mental disease; (c) determining the levels of activity of at least one protein involved in regulating intracellular GSH in vivo or in vitro in a biological sample isolated from the animal of steps (a) and (b); and (d) comparing the level of activity of step (c); wherein an alteration in the level of activity of the at least one protein indicates that the candidate agent is a modulator of the mental disorder. Preferably, the at least one protein involved in regulating the intracellular GSH level comprises GCL, preferably GCLC, GGT and/or system Xc⁻ protein. A preferred embodiment of the invention provides that the candidate agent is an agent which acts on GCLC, such as GCLM. Most preferably said candidate agent is GCLM, a functional homologue or derivative thereof.

As used herein, the term “candidate agent” refers to any molecule that is capable of increasing the level of expression of at least one gene or of normalizing the level of activity of at least one protein involved in regulating intracellular GSH. The candidate agent can be natural or synthetic molecules such as proteins or fragments thereof, organic and inorganic compounds and the like. The candidate agent may comprise more than one molecule, preferably it comprises a library of candidate agents.

Another aspect of the invention relates to a method for screening for a modulator of a mental disorder which includes step (a) combining at least one protein involved in regulating the intracellular GSH level, the protein binding partner, and a candidate agent to form a reaction mixture; and step (b) determining interaction of the protein and the protein binding partner in the presence and absence of the candidate agent. A considerable change (potentiation or inhibition) in the interaction of the protein and binding partner in the presence of the candidate agent compared to the interaction in the absence of the candidate agent indicates a potential agonist (mimetic or potentiator) or antagonist (inhibitor) of the proteins activity for the candidate agent. The components used in the screening method can be combined simultaneously or the protein can be contacted with the candidate agent for a period of time, followed by the addition of the binding partner to the reaction mixture. Complexation of the protein to its binding partner can be achieved in any type of vessel, e.g., microtitre plates, micro-centrifuge tubes and test tubes. The efficacy of the candidate agent can be assessed by using various concentrations of the agent to generate dose response curves. A control assay can also be performed by quantitating the formation of the complex between the protein and its binding partner in the absence of the candidate agent. Formation of a complex between the protein and its binding partner can be detected by using detectably labeled proteins such as radiolabeled, fluorescently labeled, or enzymatically labeled protein or its binding partner, by immunoassay or by chromatographic detection. The protein of the invention may be labeled with a specific marker and the candidate agent or a library of candidate agents labeled with a different marker. Interaction of a candidate agent with the protein or fragment thereof or the protein-binding partner can then be detected by measuring the level of the two labels after incubation and washing steps. The presence of the two labels is indicative of an interaction. Interaction between molecules can also be assessed by using real-time BIA (Biomolecular Interaction Analysis, Pharmacia Biosensor AB), which detects surface plasmon resonance, an optical phenomenon. Detection depends on changes in the mass concentration of mass macromolecules at the biospecific interface and does not require labeling of the molecules.

In preferred embodiments, the protein or its binding partner can be immobilized to facilitate separation of complexes from uncomplexed forms of the protein and its binding partner and automation of the assay. A method for immobilizing proteins on matrices involves utilizing biotin and streptavidin. For example, the protein can be biotinylated using biotin NHS N-hydroxy-succinimide), using well known techniques and immobilized in the well of streptavidin-coated plates. In one useful embodiment, a library of candidate agents can be immobilized on a sensor surface, e.g., a wall of a micro-flow cell. A solution containing the protein, functional fragment thereof, or the protein-binding partner is then continuously circulated over the sensor surface. An alteration in the resonance angle, as indicated on a signal recording, indicates the occurrence of an interaction. This technique is described in more detail in BIAtechnology Handbook by Pharmacia.

Cell-free screening methods as described above can also be used to identify agents which are capable of interacting with a protein involved in regulating the intracellular GSH level and modulate the activity of the protein. In one embodiment, the protein is incubated with a candidate agent and the catalytic activity of the protein is determined. In another embodiment, the binding affinity of the protein to a target molecule can be determined by methods known in the art.

Another aspect of the invention provides a method for screening for a modulator of a mental disorder which comprises the steps (a) administering a candidate agent to a non-human test animal which is predisposed to be affected or which is affected by a mental disease; (b) administering the candidate agent of (a) to a matched control non-human animal not predisposed to be affected or being affected by a mental disease; (c) determining the level of at least one amino acid in a plasma sample isolated from the animal of steps (a) and (b) and (d) comparing the level of the at least one amino acid of step (c); wherein an alteration in the level of the at least one amino acid indicates that the candidate agent is a modulator of the mental disorder.

Yet another aspect of the invention provides for a method for screening for a modulator of a mental disorder which comprises steps (a) administering a candidate agent to a non-human test animal which is predisposed to be affected or which is affected by a mental disease; (b) administering the candidate agent of (a) to a matched control non-human animal not predisposed to be affected or being affected by a mental disease; (c) determining the level of GCL activity and the levels of GSH in blood cells isolated from the animal of steps (a) and (b); and (d) comparing the level of GCL activity and the levels of GSH in blood cells of step (c); wherein the absence of a correlation between GCL activity and the GSH level in blood cells indicates that the candidate agent is a modulator of the mental disorder. A further embodiment of the invention is a method for screening for a modulator of a mental disorder which comprises the steps (a) administering a candidate agent to a non-human test animal which is predisposed to be affected or which is affected by a mental disease; (b) administering the candidate agent of (a) to a matched control non-human animal not predisposed to be affected or being affected by a mental disease; (c) determining the level of GGT activity and the level of cysteinyl-glycine in plasma isolated from the animal of steps (a) and (b); and (d) comparing the level of GGT activity and the level of cysteinyl-glycine in plasma of step (c); wherein a positive correlation between GGT activity and level of cysteinyl-glycine in plasma indicates that the candidate agent is a modulator of the mental disorder. The invention also provides a method for screening for a modulator of a mental disorder said method comprises the steps (a) administering a candidate agent to a non-human test animal which is predisposed to be affected or which is affected by a mental disease; (b) administering the candidate agent of (a) to a matched control non-human animal not predisposed to be affected or being affected by a mental disease; (c) determining the level of glutamate and cystine in plasma isolated from the animal of steps (a) and (b); and (d) comparing the level of glutamate and cystine in plasma of step (c); wherein a positive correlation between glutamate and cystine in plasma indicates that the candidate agent is a modulator of the mental disorder.

Another aspect of the invention relates to a method of screening for a modulator of a mental disorder, comprising determining the effect of a test substance on the activity of at least one copy of a gene involved in regulating the intracellular GSH level and/or GSH-oxidative stress-related gene expression, wherein the at least one copy of a gene has at least one polymorphism and/or at least one combination of polymorphisms which is associated with said mental disorder or predisposition therefor. Preferably, the at least one polymorphism, the at least one genotype, and/or the at least one combination of polymorphisms is defined as above.

Such methods include in vivo or cell-based and/or cell-free assays to identify compounds which are capable of interfering with the expression of at least one gene involved in regulating intracellular GSH and/or GSH-oxidative stress-related gene expression. According to one aspect of the invention, such method comprises the steps (a) determining the level of expression of at least one gene involved in regulating intracellular GSH level and/or GSH-oxidative stress-related gene expression in a sample of cells; (b) contacting the sample of cells with a test substance; (c) determining the level of expression of the at least one gene of step (a) for the sample of cells of step (b); and (d) comparing the levels of expression determined in step (a) and (c), wherein an alteration in the level of expression of the at least one gene indicates that the test substance is a modulator of the mental disorder.

According to another aspect, such method of screening for a modulator of a mental disorder comprises steps (a) administering a test substance to a non-human test animal which is predisposed to be affected or which is affected by a mental disease; (b) administering the test substance of step (a) to a matched control non-human animal not predisposed to be affected or at risk of being affected by a mental disease; (c) determining the level of expression of at least one gene involved in regulating intracellular GSH level and/or GSH-oxidative stress-related gene expression in vivo or in vitro in a biological sample isolated from the animal of step (a) and (b); and (d) comparing the levels of expression of step (c); wherein an alteration in the level of expression of the at least one gene indicates that the test substance is a modulator of the mental disorder.

A non-human test animal which is predisposed to be affected or which is affected by a mental disease may be a rat treated during the development with an inhibitor of GCL such as L-buthionine-(S,R)-sulfoximine, BSO (Rougemont M, et al. (Journal of Neuroscience Research, 70, 774-783, 2002); Castagné, V et al. (Schizophrenia Research, 60, 105, 2003), Cabungcal J H, et al. (Acta Neurobiologiae Experimentalis 63, 31, 2003)). Alternatively, a non-human test animal which is predisposed to be affected or which is affected by a mental disease may be a mice in which the GCLM gene has been knocked-out (Yang et al., J Biol Chem 277: 49446-49452, 2002).

The present invention is further described by the following non-limiting examples.

### EXAMPLES

### Example 1

### Expression of Genes Involved in Regulating GSH Level

### Fibroblast Culture

Human fibroblast cultures are established from skin biopsies of patients and from unaffected controls. Cells are grown at 37° C. and 5% CO₂ balanced air as monolayer in high glucose DMEM (GibcoBRL) containing 2% Ultroser G (Biosepra), 100 U/ml penicillin/streptomycin (GibcoBRL) and 1 mM Na pyruvate (Sigma). Primary cultures grown to confluency in two 10 cm diameter plates are frozen and kept at −150° C. until the time of analyses.

### Gene Expression

Gene expression is measured in the fibroblast cultures after three passages and at controlled density. Once the cultures reach stationary phase they are harvested with 0.2 g/L Trypsin-EDTA (GibcoBRL), and washed twice with PBS. Half of the cells are used for RNA analysis and the other half for protein analysis.

Total RNA is purified from cultured fibroblast using SV Total RNA isolation system (Promega). The RNA concentrations are measured with a fluorometric method using RiboGreen system (Molecular Probes). RNA samples of 300 ng each are reverse transcribed in Mastercycler (Eppendorf) using TaqMan transcription kit (AppliedBiosystems) and random hexamers as primers. The amplification conditions are the following: 10 minutes primer incubation at 25° C., reverse transcription at 48° C. for 30 minutes and reverse transcriptase inactivation for 5 minutes at 95° C.

Gene expression levels are measured using Taq Man chemistry and AB Prism 7000 amplifier. cDNA corresponding to 10 ng of reverse transcribed total RNA is amplified using specific primers (Table 1) at the following amplification condition: 1 cycle of 2 minutes and 50° C., 1 cycle of 10 minutes at 95° C., 50 cycles of 15 seconds at 95° C. followed by 1 minute at 60° C. The PCR fragments are then detected with labeled probes (Table 1). All detection assays were of FAM type provided by Applied Biosystems.

GAPDH is used as internal control (Applied Biosystems assay #4333764T) and a RNA pool from control cultures is used as the reference sample. The results are analyzed using AB Prism analysis program.

### Results

In fibroblast cultures obtained from skin biopsy of patients (DSM IV criteria), a decrease in the mRNA expression of the GCLM, GSS and GPX1 genes is observed in patients compared to control subjects (Table 6; the numbers represent relative level of transcription when compared to a pool of healthy subjects (n≈50) used as external standard).

TABLE 6
Expression levels of GCLM, GSS and GPX1 in fibroblast cultures from
schizophrenia patient (DSM IV criteria)

| | GCLM expression | | GSS expression | | GPX1 espression | |
|---|---|---|---|---|---|---|
| | Patient | Control | Patient | Control | Patient | Control |
| | 0.24 | 0.14 | 0.65 | 0.96 | 0.36 | 0.43 |
| | 0.30 | 0.21 | 1.56 | 0.60 | 0.77 | 0.96 |
| | 0.30 | 0.26 | 0.47 | 0.43 | | 0.81 |
| | 0.33 | 0.27 | 0.26 | 1.67 | 0.76 | 0.81 |
| | 0.35 | 0.27 | 0.83 | 1.35 | 0.72 | 0.43 |
| | 0.36 | 0.30 | 0.15 | 0.58 | 0.58 | |
| | 0.42 | 0.31 | 0.48 | 0.21 | 0.31 | 0.76 |
| | 0.44 | 0.36 | 0.65 | 0.56 | 0.28 | 0.09 |
| | 0.45 | 0.37 | 0.32 | 1.84 | 1.41 | 0.72 |
| | 0.48 | 0.40 | 0.67 | 1.16 | 1.62 | |
| | 0.48 | 0.42 | 0.50 | 0.38 | 1.37 | 0.57 |
| | 0.49 | 0.43 | 2.99 | 3.05 | 1.32 | 0.76 |
| | 0.50 | 0.45 | 0.62 | 1.76 | 0.63 | 2.27 |
| | 0.57 | 0.45 | 1.57 | 0.71 | 0.67 | 1.37 |
| | 0.59 | 0.46 | 0.65 | 4.26 | 0.84 | 0.67 |
| | 0.65 | 0.46 | 0.91 | 0.51 | | 0.69 |
| | 0.67 | 0.48 | 0.13 | 0.60 | 1.15 | |
| | 0.69 | 0.57 | 0.31 | 0.42 | 1.52 | 3.10 |
| | 0.78 | 0.60 | 0.71 | 1.01 | 0.64 | 1.45 |
| | 0.79 | 0.62 | 1.01 | 0.43 | 0.48 | 1.38 |
| | 0.85 | 0.72 | 1.96 | 0.65 | 0.73 | 0.61 |
| | 0.91 | 0.76 | 3.09 | 0.51 | 0.25 | 0.52 |
| | 0.91 | 0.76 | 2.43 | 0.49 | 0.71 | 0.56 |
| | 0.94 | 0.88 | 0.91 | 3.76 | 0.73 | 1.30 |
| | 1.04 | 0.91 | 1.24 | 0.81 | 0.60 | 1.25 |
| | 1.39 | 0.99 | 1.46 | 7.26 | 1.03 | 1.17 |
| | | 0.99 | | 0.26 | | 0.85 |
| | 1.59 | 1.32 | 0.56 | 1.30 | 0.22 | 1.25 |
| | 1.59 | 1.32 | 0.63 | 2.89 | 0.90 | 1.02 |
| | | 1.33 | | 0.68 | | 0.67 |
| | 1.71 | 1.37 | 0.68 | 1.10 | 0.99 | 0.74 |
| | 1.73 | 1.41 | 0.35 | 1.61 | 1.40 | 2.37 |
| | 1.74 | 1.41 | 0.96 | 1.24 | 0.87 | 0.81 |
| | 1.85 | 1.43 | 0.25 | 1.16 | 1.42 | 0.63 |
| | | 1.48 | | 0.28 | | 0.98 |
| | | 1.72 | | 1.89 | | 1.13 |
| | | 1.79 | | 0.30 | | 2.57 |
| | | 1.88 | | 1.50 | | 1.07 |
| | | 1.88 | | 0.31 | | 0.75 |
| | | 1.89 | | 2.41 | | 2.35 |
| | | 1.93 | | 1.29 | | 0.90 |
| | | 2.03 | | 1.06 | | 0.72 |
| | | 2.09 | | 0.86 | | 0.73 |
| | | 2.14 | | 1.92 | | 2.19 |
| | | 2.17 | | 2.28 | | 2.81 |
| | | 2.81 | | 2.44 | | 1.67 |
| | | 3.03 | | 2.85 | | 1.49 |
| | | 3.13 | | 0.49 | | |
| | | 3.24 | | 1.18 | | 2.09 |
| | | 3.31 | | 0.63 | | |
| | | 4.34 | | 1.37 | | 1.22 |
| | | 4.40 | | 5.17 | | |
| | | 4.49 | | 3.97 | | 0.58 |
| n total | 32 | 53 | 32 | 53 | 30 | 47 |
| Average | 0.82 | 1.38 | 0.86 | 1.48 | 0.82 | 1.15 |
| Average ± | 0.82 ± 0.50 | 1.38 ± 1.1 | 0.94 ± 0.76 | 1.48 ± 1.38 | 0.84 ± 0.40 | 1.15 ± 0.69 |
| Standard | | | | | | |
| deviation | | | | | | |
| % decrease | 40.8% | | 36.7% | | 26.4% | |
| in mRNA | | | | | | |
| expression | | | | | | |
| p value | ≦0.002 | | ≦0.02 | | ≦0.01 | |

GCLM mRNA expression reveal two sub-populations those having a level of expression similar to controls (high GCLM n=8, 24% of the total patient pool) and those having much lower levels (low GCLM n=24, 76%).

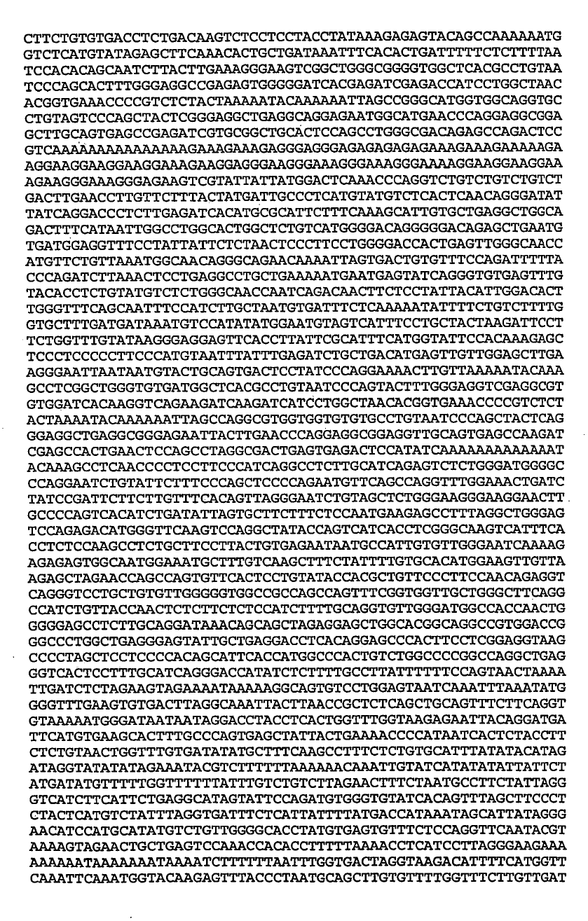

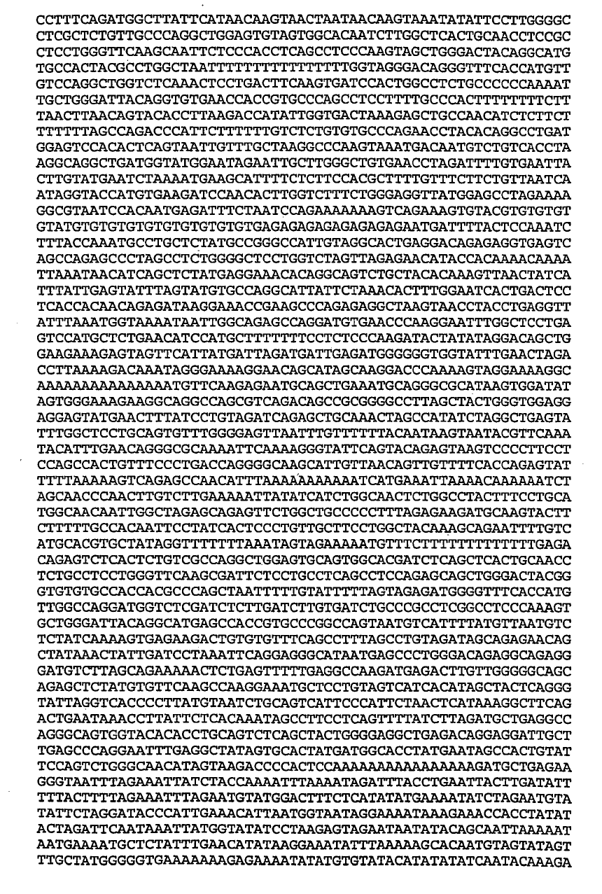

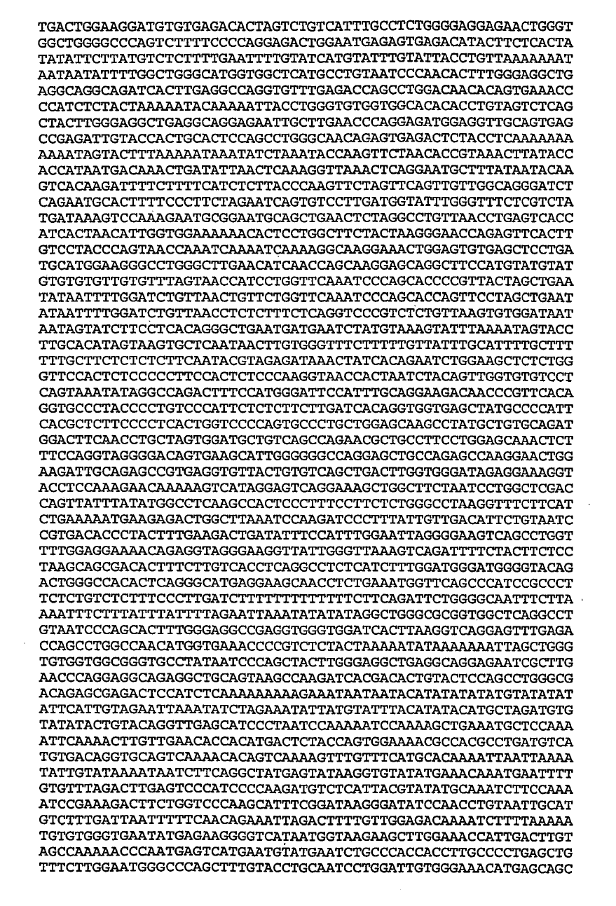

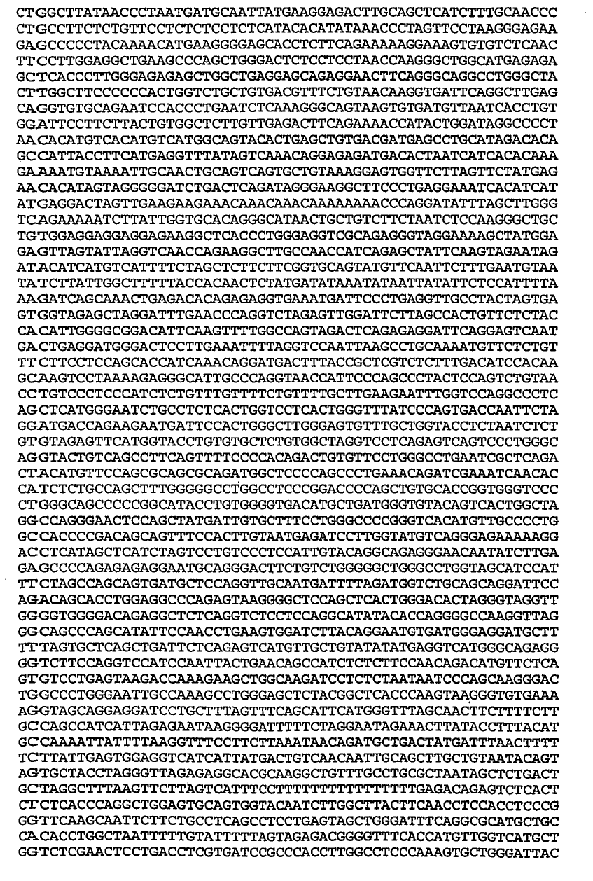

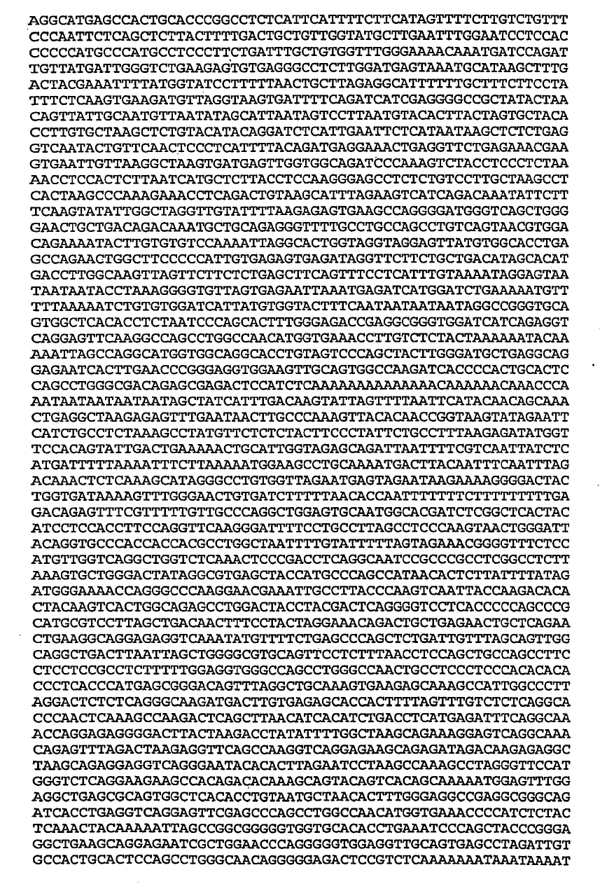

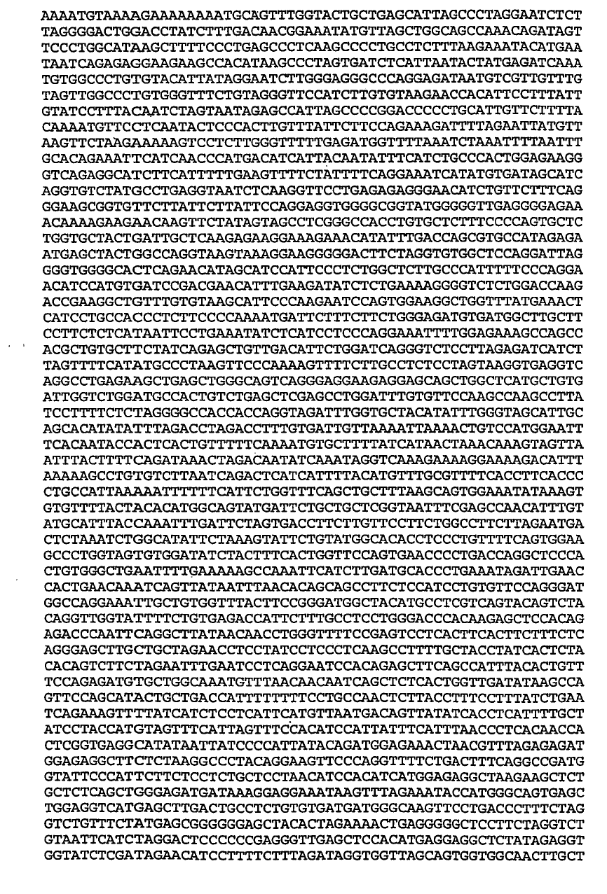

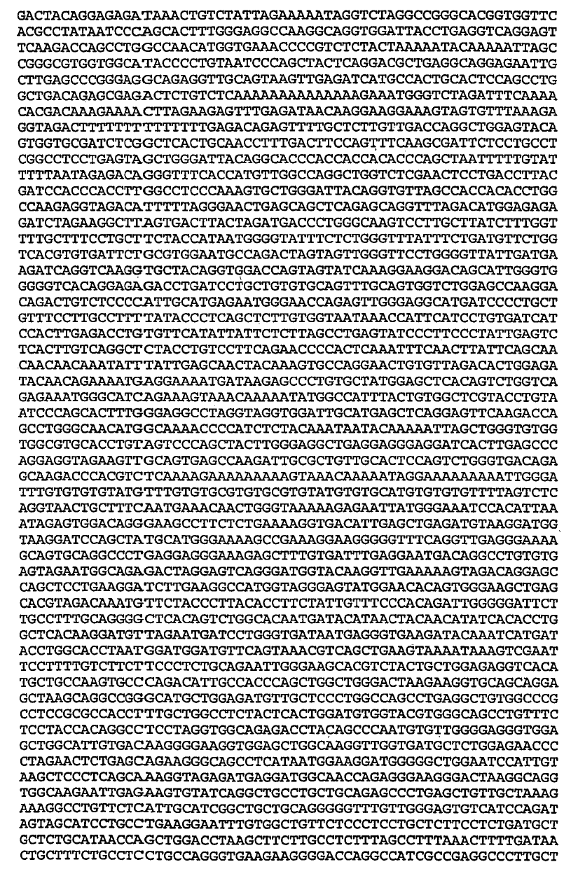

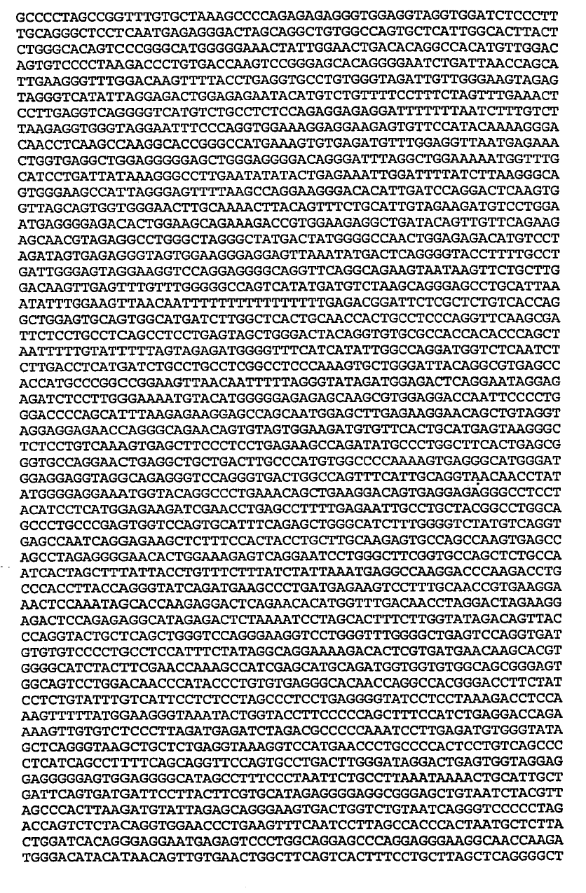

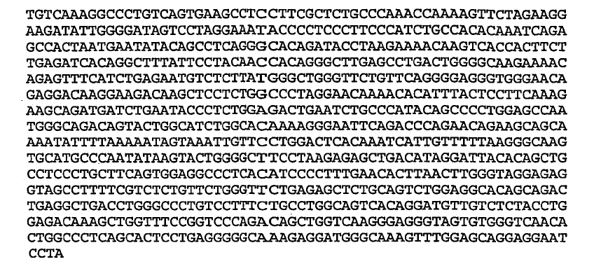

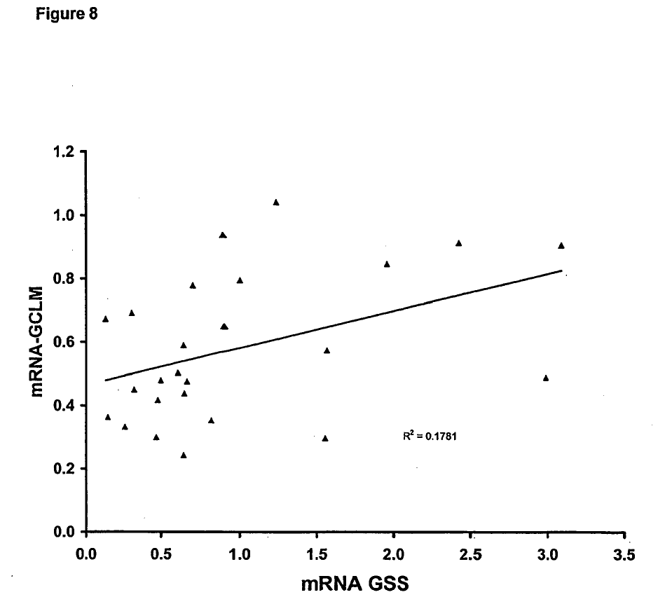

Furthermore, a positive correlation between GCLM and GSS is found in fibroblasts isolated from patients: patients with low GCLM expression levels, show also low levels of GSS expression (r2=0.18; p≦0.05) (FIG. 8).

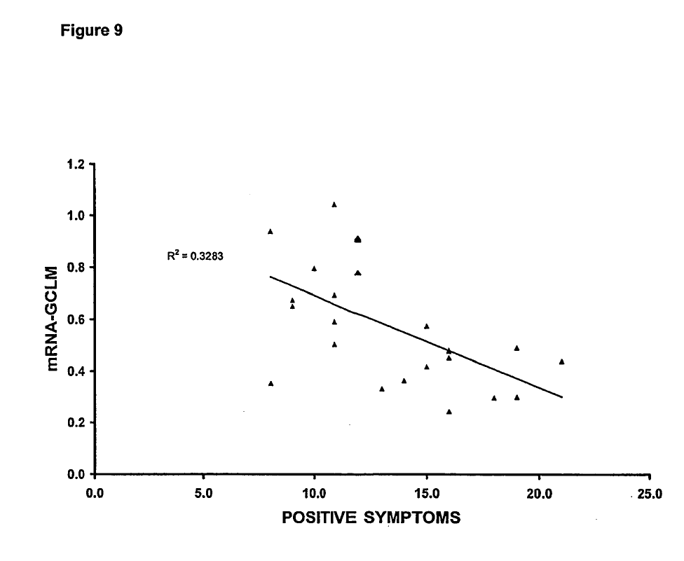

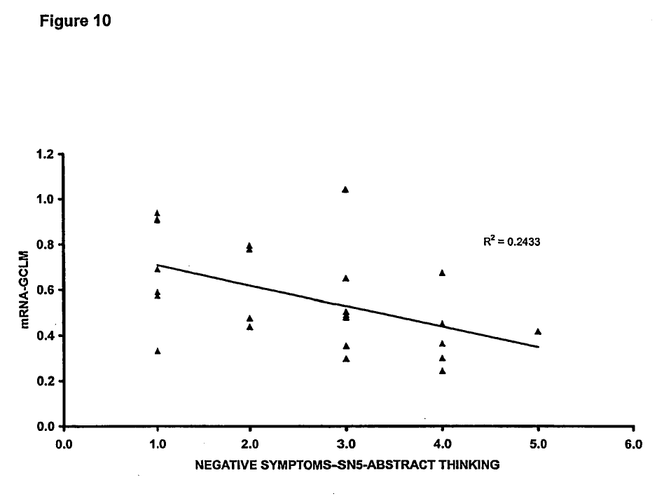

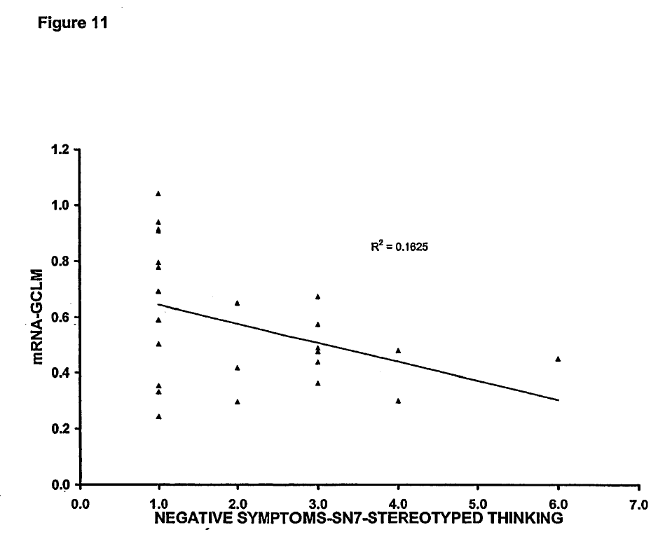

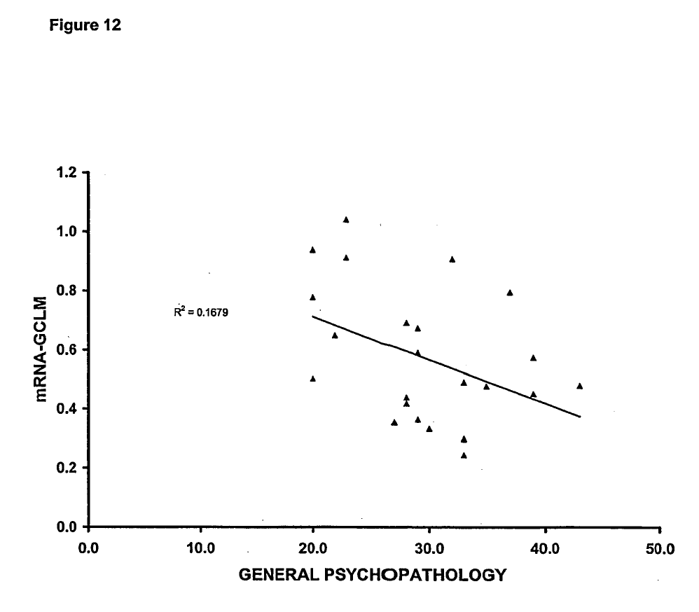

In addition, it is found that the level of expression of GCLM gene in fibroblasts correlates with clinical scores of symptoms of a patient (FIG. 9 to 12). Low expression levels of GCLM correlates negatively with clinical scores of the positive symptoms (FIG. 9, r2=0.33, p≦0.05), negatively with items 5 and 7 of the negative scale (FIGS. 10 and 11, thought disorders, SN5: r2=0.24, p=0.02; and stereotyped thinking, SN7: r2=0.16, p≦0.05) and negatively with the general psychopathology scale (FIG. 12, r2=0.17). Patients with the most severe symptoms (high score) have the lowest GCLM mRNA expression, while those with more normal expression have a lighter symptom score.

### Example 2

### GSH Levels in Blood

### Blood Preparation

Blood is collected from a patient by venipuncture between 7 and 8:30 AM under restricted activity conditions and fasting from the previous midnight. 18-20 ml blood is allowed to drop into a ice-cold Vacutainer-tubes coated with EDTA (Becton Dickinson) and the hemoglobin is quantified. All following preparations are performed on ice or at 4° C. An aliquot of whole blood is sampled and frozen at −80° C. until analysis of glutathione (GSH) content. The rest of blood is centrifuged at 3000 g, 5 min, 4° C.; the pellet, corresponding to blood cells, is washed 2 times with 0.9% NaCl and stored at −80° C. until analysis. The supernatant, corresponding to the plasma, is recovered, sampled in aliquots and kept at −80° C. until analysis.

### Glutathione (GSH) Determination

The GSH levels in blood cells, plasma or fibroblasts are determined using a diagnostic kit (Calbiochem). The method is based on a colorimetric assay of a chromophoric thione formed specifically between the reagent and GSH.

### Result

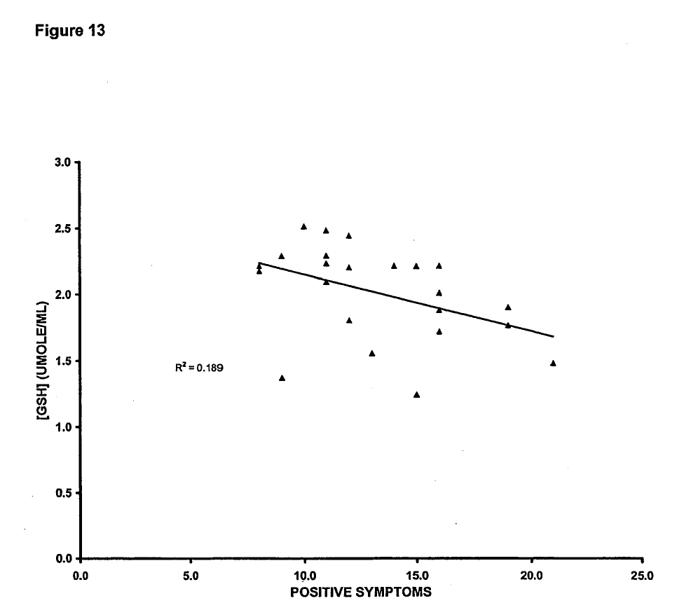

The GSH levels in blood cells of patients is negatively correlated with the clinical score of the positive symptoms (FIG. 13, r2=0.19, p=0.008); i.e. the lower the GSH levels, the higher the psychopathology.

### Example 3

### Activity of Proteins Involved in Regulating GSH Level

### Fibroblast Preparation for Biochemical Assays

Cultures of fibroblast (isolated and cultured as described in Example 1) 4 plates of 10 cm diameter, confluent cell layer) are collected after 3 passages. The cells are removed from the dishes by trypsin treatment, washed, resuspended in 4 ml phosphate buffer (0.1 M, pH 7.4) and sonicated. Aliquots from this homogenate are kept at −0° C. for GSH and protein determination. The rest of the homogenate is centrifuged at 5000 g for 10 min at 4° C. The supernatant is sampled in 100 μl aliquots and used for GSH-related enzymes activity determination.

### Protein Determination

The protein levels of fibroblasts are determined using the Biorad Kit with the Advanced Protein Assay reagent.

### System Xc⁻ Activity in Fibroblasts: Uptake of [³⁵S] Cystine

The system Xc⁻ activity are measured as [³⁵S] cystine uptake, as described by Bannai S and Kitamura E (Journal of Biological Chemistry 255, 2372-2376, 1980) with the following modifications. Cultures of fibroblast (isolated and cultured as described in Example 1), 3 plates of 3.5 cm diameter, confluent cell layer) are used after 3 passages. The cells are rinsed three times in prewarmed (37° C.) 10 mM phosphate-buffered saline, pH 7.4, containing 0.01% CaCl₂, 0.01% MgCl₂, 0.1% glucose, 137 mM NaCl and 3 mM KCl. After rinsing, the cells are incubated in 1 ml of the prewarmed uptake medium for 2 min at 37° C. The uptake medium consisted of the same buffer used to rinse the cells and labeled cystine (2 μCi/ml, and 0.05 mM, Amersham). The incubations are terminated by rapidly rinsing the dishes three times in ice-cold phosphate-buffered saline. Then 0.5 ml of 0.5 N sodium hydroxide is added to each dish. The cells are collected and sonicated. 300 μl of the solution is mixed with 3 ml of scintillation liquid containing 1% of Triton X-100 and the radioactivity measured. An aliquot of the solution is used for protein assay. All determinations are averages of triplicate samples that varied ±10%.

### γ-Glutamylcysteine Ligase (GCL) Activity in Blood Cells

Blood cells are prepared as described in Example 2. GCL activity is determined as described by Gegg et al. (Analytical Biochemistry, 304, 26-32, 2002) with the following modifications: hemolyzed blood cells derived from 500 μl blood is taken in Tris-buffer (100 mM, pH 8.2) and centrifuged through a microcon centrifugal filter device with a 10-kDa molecular cut-off filter (Millipore Ultrafree-MC 10000) at 14000 g for 15 min at 4° C. This step removes amino acids, cofactors, small molecules (GSH) which interfere with the reaction. The precipitate is taken in Tris-buffer (100 mM, pH 8.2) and aliquot of 60 μl treated with 90 μl of a Tris-buffer solution containing ATP (10 mM), α-aminobutyric acid (10 mM, Fluka), L-[U-¹⁴C]-glutamate (10 mM, specific activity 3 MBq/mmol, Amersham). Following incubation at 37° C. for 20 min, the reaction is stopped by the addition of 12 μl of 80% sulfosalicylic acid. Denaturated protein is removed by centrifugation at 17.000 g for 6 min and the supernatant is filtered (Millipore, HV, 0.45 μm) before analysis with HPLC (Hewlett Packard model 1090 chromatograph). The radioactive product ¹⁴C-γ-glutamyl-aminobutyric acid is separated on a HPLC column (125×4 mm), packed with Hypersil (C18, 5.0 μm) with a linear gradient of 2-80% mobile phase B (0.25% trifluoroacetic acid in acetonitrile/water (9/1, v/v)) in mobile phase A (0.25% trifluoroacetic acid in water) during 40 min at a flow rate of 0.5 ml/min. The radioactive product is detected by using a flow scintillation analyser (Canberra Packard Flow-one Beta 500) which is combined on-line to the HPLC. The ¹⁴C-γ-glutamyl-aminobutyric acid eluted at 19 min. The quantification of the radioactive product is based on area measurements. To assess the specificity of the assay, the samples are preincubated with 500 μM L-buthionine-SR-sulfoximine (BSO), a specific inhibitor of GCS and 10 mM ATP for 5 min at room temperature and then assayed as described above in the presence of BSO.

### γ-Glutamyltranspentidase (GGT) Activity in Plasma

GGT activity is measured using a diagnostic kit purchased from Sigma Diagnostics. GGT catalyses the formation of 5-amino-2-nitrobenzoate from γ-glutamyl-3-carboxy-4-nitroanilide at 37° C. Enzyme activity is measured at an absorbance of 405 nm.

### Results

### System Xc⁻

The activity of system Xc⁻ in fibroblast of patients (0.73 pmole/μg proteine/min) is higher (+16%, p=0.05) than the one of the control subjects (0.63 pmole/μg proteine/min; Table 7).

TABLE 7
Activity of system X_{c}⁻ in fibroblasts

| System Xc⁻ activity (pmol/μg protein) | |
|---|---|
| Patient | Control |

| | | | |
|---|---|---|---|
| 0.63 | 0.85 | 0.38 | 0.69 |
| 0.67 | 0.89 | 0.48 | 0.60 |
| 1.07 | 0.83 | 0.65 | 0.44 |
| 0.67 | 0.74 | 0.56 | 0.65 |
| 1.19 | 0.68 | 0.78 | 0.56 |
| 0.65 | 0.50 | 0.65 | 0.73 |
| 0.67 | 0.83 | 0.32 | 0.58 |
| 0.74 | | 0.55 | 1.04 |
| 0.80 | | 0.54 | 1.21 |
| 0.41 | | 0.56 | 0.50 |
| 0.78 | | 0.60 | 0.47 |
| 0.71 | | 0.90 | 0.42 |
| 1.01 | | 0.73 | 0.66 |
| 0.78 | | 0.85 | 0.30 |
| 0.61 | | 0.50 | 0.53 |
| 0.35 | | 0.78 | 0.63 |
| 0.46 | | 0.61 | 0.87 |
| 0.89 | | 0.47 | 0.90 |
| 1.26 | | 0.96 | 0.47 |
| 0.31 | | 0.50 | 0.43 |

| | | |
|---|---|---|
| n total | 27 | 40 |
| Average | 0.73 | 0.63 |
| Average ± | 0.73 ± 0.24 | 0.63 ± 0.20 |
| Standard | | |
| deviation | | |
| % increase | 16% | |
| in system | | |
| Xc activity | | |
| p value | ≦0.05 | |

### GCL

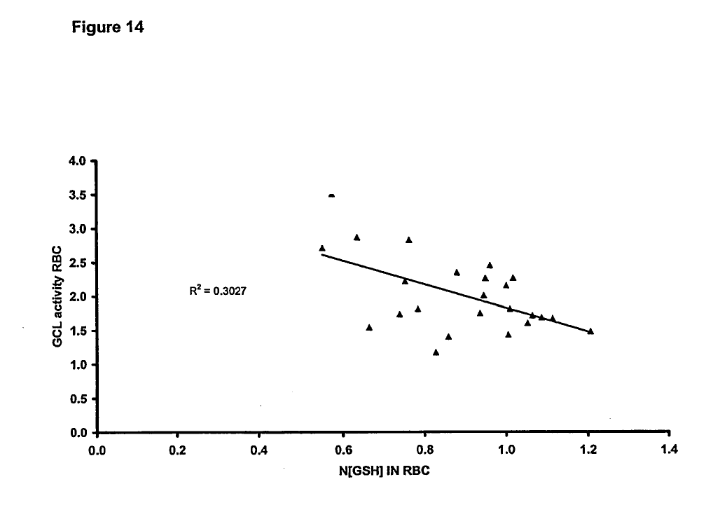

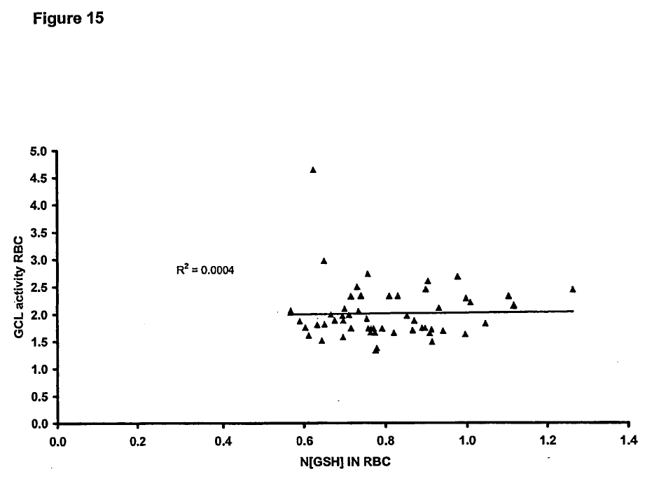

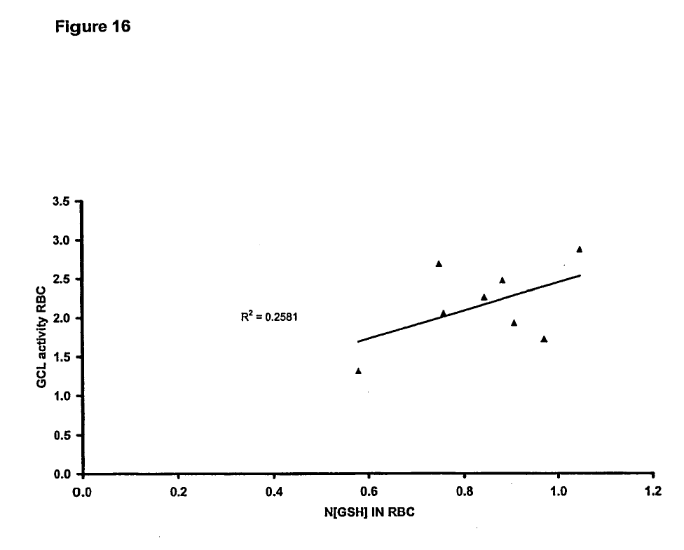

A negative correlation between the GCL activity and GSH levels in blood cells of patients with low GCLM gene expression (FIG. 14; r2=0.30), which is not the case for the group of controls (FIG. 15) and of the patients with high GCLM gene expression (FIG. 16). Thus, an increased feedback inhibition of the GCL activity with increasing GSH levels is observed in blood cells isolated from patients with low GCLM expression.

### GGT

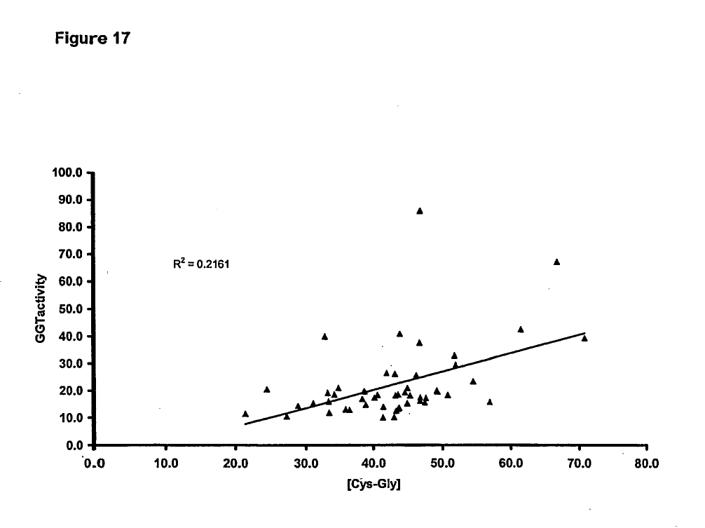

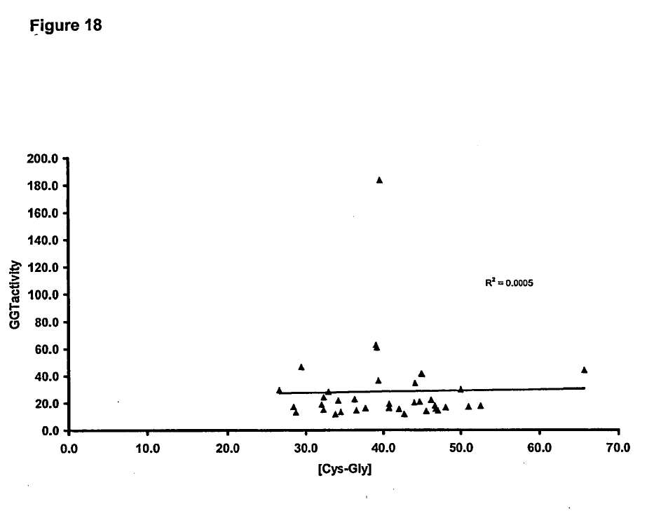

In plasma isolated form control subjects, there is a positive correlation of GGT activity and cysteinyl-glycine (Cys-Gly; the direct product of GGT degradation of GSH) levels in control subjects (FIG. 17; r2=0.22), which is absent in patients (FIG. 18; r2=0005). The concentration of Cys-Gly is determined as described below in Example 4.

### Example 4

### Amino Acid Analysis

### Amino Acid Analysis in Plasma

The following free amino acids are quantified in plasma as described in Slocum et al. (In “Techniques in diagnostic human Biochemical Genetics”. Hommes Edt. 1991, pp 87-126): Taurine (Tau), Aspartic acid (Asp), Hydroxyproline (Hyp), Threonine (Thr), Serine (Ser), Asparagine (Asn), Glutamic acid (Glu), Glutamine (Gln), Proline (Pro), Glycine (Gly), Alanine (Ala), Citrulline (Cit), Aminobutyric acid (Abu), Valine (Val), Cystine (Cyt), Methionine (Met), Isoleucine (Ile), Leucine (Leu), Tyrosine (Tyr), Phenylalanine (Phe), Ornithine (Orn), Lysine (Lys), 1-CH₃-Histidine (1-CH₃-His), Histidine (His), 3-CH₃-Histidine (3-CH₃-His), Arginine (Arg). In brief, the plasma containing (S)-2-Aminoethyl-1-cysteine and d-glucosaminic acid (1.25 mM) as internal standards is deproteinated by 5-sulfosalicylic acid and kept at −80° C. until analysis. The solution is injected into an amino acid analyser (Beckman, 6300 Model; column Lithium, 10 cm, Beckman 338051) and the amino acids detected by post-column reaction with nihydrine.

### Thiols Analysis in Plasma

The following thiol containing amino acids and peptides are quantified in plasma as described in Jacobsen et al. (Gen Clin Chim, 873-881, 1994): Cysteine (total, i.e. reduced and oxidized form), Homocysteine and Cysteinyl-glycine. In brief, the thiols are reduced and/or decoupled from peptides by reaction with Tris(2-carboxyethyl)phosphine. After deproteinization with perchloric acid, the thiols are derivatized with 7-fluorobenzofurazane-4-sulfonic acid (SBD-F). The SBD-F derivatives are subsequently analyzed by HPLC followed by fluorometric detection.

### Results

Cysteine is the rate-limiting precursor of glutathione and homocysteine is linked to the metabolism of methionine, an essential amino acid leading to the formation of cysteine. Among the 28 amino acids analysed, the plasmatic level of cysteine (Cys, total level), and homocysteine (Hcys), are higher (+8.0%, p=0.02, and +22.6%, p=0.01, respectively) in the entire group of patients (n=37; in pmole/L Cys 272.0±41.5; Hcys: 10.0±3.6) compared to controls (n=51: in μmole/L Cys 252.0±44.4; Hcys: 8.2±2.3) (Table 8). In the sub-group of patients with low GCLM mRNA (n=25), cysteine and homocysteine levels are still significantly higher than in controls (+12%, p=0.03 and +33%, p=0.02, respectively).

TABLE 8
Levels of cysteine and homocysteine in plasma of patients and controls

| | Cysteine levels (μM) | | Homocysteine levels (μM) | |
|---|---|---|---|---|
| | Patient | Control | Patient | Control |
| | | 217.8 | | 6.2 |
| | | 395.0 | | 13.6 |
| | 336.0 | | 13.5 | |
| | 300.0 | 215.8 | 9.4 | 6.0 |
| | | 221.0 | | 8.5 |
| | 292.3 | 252.3 | 9.7 | 7.9 |
| | 251.3 | 218.0 | 6.6 | 11.1 |
| | 291.2 | | 7.5 | |
| | 347.0 | 259.1 | 16.4 | 8.5 |
| | 370.0 | 246.0 | 11.1 | 8.2 |
| | 256.4 | | 10.6 | |
| | | 195.0 | | 10.7 |
| | 292.2 | 302.3 | 11.4 | 8.3 |
| | | 229.0 | | 7.9 |
| | 287.0 | | 8.9 | |
| | 329.4 | 273.0 | 11.7 | 6.7 |
| | 237.5 | 286.0 | 23.8 | 6.9 |
| | 182.0 | 194.0 | 8.7 | 7.2 |
| | 300.1 | 259.6 | 8.6 | 9.3 |
| | 282.1 | 229.0 | 10.3 | 8.6 |
| | 241.1 | 203.9 | 6.7 | 5.7 |
| | 220.9 | 204.5 | 8.4 | 7.0 |
| | | 299.8 | | 10.5 |
| | | 222.1 | | 5.2 |
| | 298.4 | 282.2 | 8.8 | 10.0 |
| | | 282.5 | | 13.9 |
| | 244.0 | 307.6 | 9.3 | 8.2 |
| | 287.0 | | 10.1 | |
| | | 246.1 | | 3.7 |
| | 358.4 | 230.9 | 19.4 | 9.9 |
| | 200.5 | 228.1 | 5.7 | 10.0 |
| | | 215.3 | | 5.3 |
| | 268.5 | 272.6 | 8.7 | 6.5 |
| | 244.4 | 232.6 | 9.7 | 7.0 |
| | 249.0 | 257.8 | 10.5 | 6.8 |
| | 258.3 | 383.1 | 12.5 | 14.3 |
| | 258.5 | 161.6 | 7.3 | 7.2 |
| | 261.8 | 221.4 | 10.4 | 6.6 |
| | 227.4 | 248.8 | 8.3 | 5.5 |
| | 242.3 | | 6.9 | |
| | 246.4 | 245.8 | 8.7 | 10.7 |
| | 255.8 | 255.7 | 5.8 | 8.4 |
| | 257.3 | 301.0 | 8.9 | 8.0 |
| | 261.2 | 300.0 | 8.6 | 9.2 |
| | 311.4 | 313.8 | 8.4 | 10.6 |
| | 241.3 | 212.6 | 7.7 | 6.0 |
| | 277.6 | 237.5 | 11.6 | 6.1 |
| | | 239.7 | | 5.5 |
| | | 270.7 | | 7.9 |
| | | 237.3 | | 5.3 |
| | | 231.4 | | 7.3 |
| | | 256.4 | | 9.0 |
| | | 215.6 | | 7.6 |
| | | 221.2 | | 8.9 |
| | | 264.9 | | 8.2 |
| | | 321.8 | | 11.3 |
| | | 232.0 | | 7.8 |
| n total | 37 | 51 | 37 | 51 |
| Average | 272.0 | 252.0 | 10.0 | 8.2 |
| Average ± | 272.0 ± 41.5 | 252.0 ± 44.4 | 10.0 ± 3.6 | 8.2 ± 2.3 |
| Standard | | | | |
| deviation | | | | |
| % increase | 8.0% | | 22.6% | |
| in levels | | | | |
| p value | ≦0.02 | | ≦0.01 | |

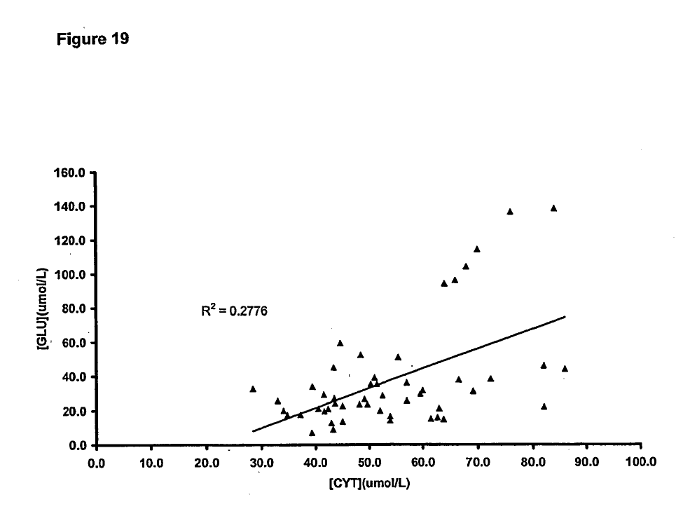

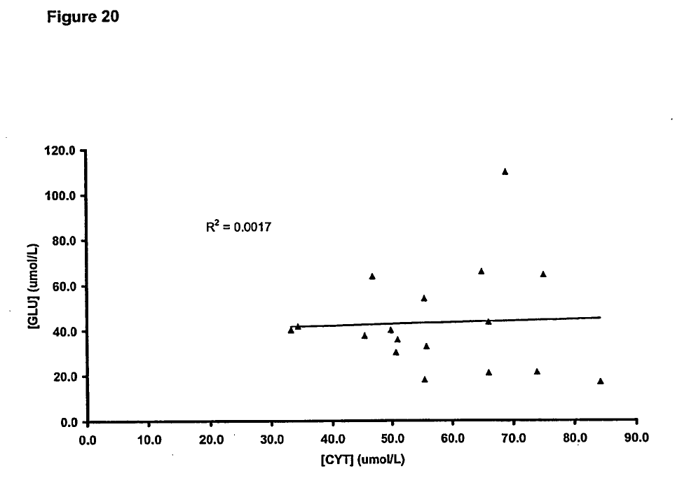

Furthermore it is observed that the plasmatic levels of cystine and glutamate are positively correlated in control subjects (FIG. 19, r²=0.28) while this correlation is abolished in the subgroup of patients with low GCLM gene expression (FIG. 20, r²=0.002).

### Example 5

### Association of the Polymorphisms in the Glutathione-Related Genes with Schizophrenia

### Introduction

Gene expression study for 12 genes involved in the glutathione metabolism using fibroblast cultures obtained from the skin biopsy from schizophrenia patients and a control group show a significant decrease in two genes directly involved in GSH synthesis: glutamate-cysteine ligase, modifier subunit (GCLM) and glutathione synthetase (GSS). In order to test if these reduced levels of the mRNAs corresponding to these two genes are due to the differences in the genes themselves or to an epigenic factor, all the subjects used in the expression studies were genotyped and an association study for polymorphisms known for these genes performed. The aim was to detect a possible relationship between the particular alleles/haplotypes of the candidate genes and the disease.

16 single nucleotide polymorphisms (SNP) were selected from publicly available databases SNP Consortium http://snp.cshl.orq and NCBI http://www.ncbi.nlm.nih.gov. NCBI annotated SNP numbers, their alternative alleles and their positions and distances from the beginning of the genes are shown in Table 9. The SNP positions are based on the information found in the NCBI graphic representation of the appropriate contig.

TABLE 9

| | | | | SNP location | SNP |
|---|---|---|---|---|---|
| Gene | Location | SNP# | Allele | in the gene | position |
| GCLM | 1p22.1 | rs2235971 | G/A | 30250 | 3′ region |
| | | rs3170633 | A/G | 23638 | 3′ region |
| | | rs2064764 | A/G | 16623 | intron 6 |
| | | rs769211 | T/G | 14859 | intron 6 |
| | | rs718873 | T/C | 3057 | intron 1 |
| | | rs718875 | T/C | 2946 | intron 1 |
| | | rs2301022 | A/G | 2088 | intron 1 |
| GSS | 20q11.2 | rs3746450 | C/A | 35013 | 3′ region |
| | | rs725521 | T/C | 27590 | 3′ region |
| | | rs1801310 | A/G | 26587 | intron 12 |
| | | rs2236270 | T/G | 20446 | intron 9 |
| | | rs2236271 | A/C | 19761 | intron 8 |
| | | rs2273684 | T/G | 13835 | intron 5 |
| | | rs734111 | C/A | 9865 | intron 3 |
| | | rs2025096 | A/G | 3601 | intron 1 |
| | | rs3761144 | C/G | −474 | 5′ region |

Association study was done for 7 SNPs corresponding to GCLM gene and 9 SNPs to GSS gene. These genes have lower expression in the patients compared to controls. Also included was one SNP for glutamate-cysteine ligase, catalytic subunit (GCLC), a peptide that forms a heteroduplex with the GCLM giving rise to a rate-limiting enzyme glutamate-cysteine synthetase (GCL).

### Method

### Genotyping

DNA was purified from the peripheral blood obtained from 47 patients and 115 controls using Nucleon BACC kit (Amersham Pharmacia Biotech). The genotyping for 16 single nucleotide polymorphisms (SNP) was performed by Methexis Genomics (Gent, Belgium) using MALDI-TOF mass spectrometry.

### Template Amplification

Following genomic extraction, 5 ng of genomic DNA containing the SNP site of interest was amplified in a 5 μl volume using a 384-microtiter plate format. Arctic shrimp alkaline phosphatase was added to samples, which were then incubated for 20 minutes at 37 degrees Celsius. This dephosphorylated any residual amplification nucleotides, preventing their future incorporation and interference with the primer extension assay. The MassEXTEND primer, DNA polymerase, and a cocktail mixture of deoxynucleotides (dNTPs) and dideoxynucleotide triphosphates (ddNTPs) were added to initiate the hME primer extension reaction. This reaction generated allele-specific primer extension products that are generally one-to-four bases longer than the original MassEXTEND primer. A common MassEXTEND primer that identifies both alleles was hybridized directly or closely adjacent to the polymorphic site. Nucleotide mixtures were selected to maximize mass differences for all potential MassEXTEND products. Appropriate dNTPs were incorporated through the polymorphic site until a single ddNTP was incorporated, and the reaction terminated.

Since the termination point and number of nucleotides are sequence-specific, the mass of the extension products generated could be used to identify the possible variants without errors. Following the extension reaction, MassEXTEND clean resin was added to the reaction to remove extraneous salts that interfere with MALDI-TOF analysis. Fifteen μl of sample were then transferred from the 384-microtiter plate and spotted onto the pad of the 384-SpectroCHIP™ bioarray The SpectroCHIP was placed into the MALDI-TOF, and the mass and correlating genotype were determined in real time with MassARRAY RT™ software.

### Results

### Individual SNP Analysis

Individual marker genotype association was tested for each gene separately. After removal of ill conditioned variables (SNP genotype effects with excessive or zero variances) we found highly significant joint effects of SNPs in gene GCLM and nearly significant effects in gene GSS (Table 10). Genes GCLC did not furnish results anywhere near significance (p>>0.05).

TABLE 10
Results of logistic regression analysis for genes GCLM and GSS.

| | |
|---|---|
| Odds ratio | 95% confidence limits |

| SNP | estimate | lower | upper | p-value |
|---|---|---|---|---|
| rs2235971 | 1.4 | 0.36 | 5.41 | 0.6267 |
| rs3170633 | 3.16 | 0.66 | 15.47 | 0.1551 |
| rs769211 | 0.93 | 0.2 | 4.45 | 0.9314 |
| rs2301022 | 0.54 | 0.15 | 1.9 | 0.3397 |
| GCLM regr. | — | — | — | 0.0009 |
| rs2236270 | 0.43 | 0.09 | 2.12 | 0.2975 |
| rs2273684 | 1.57 | 0.19 | 13.01 | 0.6768 |
| rs734111 | 2.36 | 0.27 | 20.55 | 0.4361 |
| rs2025096 | 1.57 | 0.64 | 3.83 | 0.3181 |
| rs3761144 | 10.88 | 1.01 | 117.66 | 0.0495 |
| GSS regr. | — | — | — | 0.0653 |

Evidently, with the exception of SNP rs3761144 in gene GSS, each individual SNP did not exert much of an effect of its own but the joint effects of SNPs were strong, at least for gene GCLM, for which the joint effects of SNPs rs2235971, rs3170633, rs769211, and rs2301022 were highly significant (p=0.0009).

Logistic regression analysis for GCLM SNPs in combination with other variables (age, gender and gene expression level) showed their high joint effect on disease outcome (p=0.0006). The strongest result (p=0.0009) was obtained for SNP rs3170633 (Table 11).

TABLE 11

| Parameter | Odds Ratio | Upper | Lower | p-val |
|---|---|---|---|---|
| AGE (months) | 0.998472 | 1.005379 | 0.991612 | 0.6637 |
| LNGENE GCLM | 0.236392 | 0.93542 | 0.059739 | 0.0399 |
| GENDER | 1.320465 | 10.376054 | 0.168044 | 0.7916 |
| rs2235971 | 0.264751 | 11.476123 | 0.006100 | 0.4894 |
| rs3170633 | 0.024751 | 0.965534 | 0.000634 | 0.0478 |
| rs769211 | 2.526676 | 295.058025 | 0.021637 | 0.7027 |
| rs2301022 | 1.857265 | 56.841212 | 0.060685 | 0.7228 |

7 df Chi-sq p-value=0.000611

Regression analysis for each SNP separately in combination with other variables showed that only the expression level and SNP genotype significantly predict disease outcome. This was the case for all SNPs with p<0.05. Again the strongest effect was found for SNP rs3170633 (Table 12).

TABLE 12
Results of multiple logistic regression analysis for gene
GCLM and single SNP rs3170633 as independent variable.

| | 95% confidence interval | | | |
|---|---|---|---|---|
| Parameter | Odds Ratio | Lower | Upper | p-value |
| AGE MONTHS | 1.00 | 1.00 | 1.01 | 0.7163 |
| LN GCLM RNA | 4.03 | 1.27 | 12.84 | 0.0183 |
| GENDER | 1.18 | 0.25 | 5.48 | 0.8314 |
| rs3170633 | 8.42 | 1.81 | 39.18 | 0.0066 |

For gene GSS, only expression levels showed predictive effects on outcome with p-values generally well below 0.05, but regression analysis with all three input variables was not significant for any of the SNPs. The best result (p=0.0578) was obtained for SNP rs3761144, with p=0.0133 for the effect of gene GSS expression level. However, correction for multiple testing renders these results non-significant.

When SNPs were analyzed independently one by one, the strongest effect was seen for SNP rs3170633 in gene GCLM with p=0.0037 (Table 13). When genotype GG was compared against the other two genotypes combined, GG was associated with an odds ratio (OR) of 2.95 (95% limits of 1.34, 6.47). That is, individuals with the GG genotype have an approximately 3 times higher risk of being ill than other individuals.

TABLE 13
Genotype analysis for SNP rs3170633 in gene GCLM

| | | |
|---|---|---|
| SNP | No. observed | Proportion |

| genotype | patients | controls | patients | controls |
|---|---|---|---|---|
| AA | 1 | 18 | 0.03 | 0.19 |
| AG | 13 | 43 | 0.35 | 0.45 |
| GG | 23 | 34 | 0.62 | 0.36 |
| sum | 37 | 95 | 1 | 1 |

### Haplotype Analysis

Based on significant or nearly significant results of genotype analyses, haplotype analysis was carried out for genes GCLM and GSS. As shown in Table 14, we obtained the same number of common haplotypes for each of the two genes. Haplotype frequencies were significantly different between patients and control individuals for genes GCLM (p=0.0228) and GSS (p=0.0004). Haplotypes with strongest effects were GGGA (OR=2.19) in gene GCLM and TGAGG (OR=1.69) and CTCGC (OR=1.58) in gene GSS.

TABLE 14
Haplotype frequencies for genes GCLM and GSS. OR = odds ratio

| Gene GCLM | | | | | | | |
|---|---|---|---|---|---|---|---|
| Haplo- | | | | Gene GSS | | | |
| type | patients | control | OR | Haplotype | patients | control | OR |
| AAGG | 0.023 | 0.057 | 0.40 | CGCGC | 0 | 0.151 | 0 |
| AATG | 0.172 | 0.291 | 0.51 | CTCAC | 0.128 | 0.241 | 0.46 |
| GGGA | 0.442 | 0.265 | 2.19 | CTCGC | 0.255 | 0.178 | 1.58 |
| GGGG | 0.346 | 0.358 | 0.95 | TGAGG | 0.538 | 0.408 | 1.69 |
| rare | 0.017 | 0.029 | — | rare | 0.079 | 0.023 | — |
| sum | 1 | 1 | — | sum | 1 | 1 | — |

Frequencies of the most common haplotypes were significantly different between patients and control individuals for genes GCLM (p=0.0228) and GSS (p=0.0004). Haplotypes with strongest effects were GGGA (OR=2.19) in gene GCLM and TGAGG (OR=1.69) and CTCGC OR=1.58) in gene GSS.

When haplotypes were estimated for the nine SNPs in genes GCLM and GSS jointly, haplotype frequencies were significantly different between the two groups of individuals with p=0.000062 (detailed results not shown). The haplotype with strongest effect was GGGA-CTCGC OR=4.04; an additional three haplotypes showed ORs exceeding 2). For independent action of the two component haplotypes the predicted odds ratio was 2.19×1.58=3.46, that is, the joint haplotype has a stronger effect than predicted. However, approximate analyses (not shown) indicate that this increase in the OR is not statistically significant, so the two component haplotypes contribute independently to the risk for disease.

As haplotypes can be recognized with certainty when they occur in the homozygous state, for those haplotypes with OR>1.50 in Table 10 we determined the proportion of individuals who are homozygous versus those with other haplotypes (Table 15). Evidently, only the haplotypes GGGA and TGAGG exerted a relatively strong effect when in the homozygous state (sometimes called a diplotype) but these effects are only marginally significant.

TABLE 15
Numbers of individuals who are homozygous for each
of three haplotypes with OR > 1.50.

| GGGA | | | | CTCGC | | | | TGAGG | | | |
|---|---|---|---|---|---|---|---|---|---|---|---|
| genotype | P | C | OR | genotype | P | C | OR | genotype | P | C | OR |
| homoz. | 7 | 8 | 2.81 | homoz. | 2 | 2 | 3.03 | homoz. | 10 | 13 | 2.68 |
| other | 33 | 106 | | other | 37 | 112 | | other | 29 | 101 | |
| sum/p | 40 | 114 | 0.068 | sum/p | 39 | 114 | 0.287 | sum/p | 39 | 114 | 0.040 |

The last row shows the sum or the p-value associated with the given OR.

### Prediction

Predictions based on SNP genotypes associated alone with disease are not very good (Table 16). This is presumably due to various other factors influencing disease. Power (sensitivity) never exceeds 50%, and the specificity is in the neighborhood of 80% or less. Nonetheless, as judged by the total probability of correct prediction, genotypes tend to increase success of prediction from 50% for random prediction up to 73% for some of haplotypes in the homozygous state. Thus, these parameters can be very useful for prediction of the disease outcome but in combination with other factors not linked to these two genes.

TABLE 16
Prediction parameters for different predictors of disease.

| | sensi- | speci- | Total | |
|---|---|---|---|---|
| Predictor | tivity | ficity | correct | p-value |
| GCLM, 4 SNPs | 0.37 | 0.77 | 0.66 | 0.0009 |
| GSS, 5 SNPs | 0.30 | 0.77 | 0.66 | 0.0495 |
| SNP rs3170633, genotype GG | 0.40 | 0.81 | 0.64 | 0.0104 |
| GGGA haplotype, homozygous | 0.18 | 0.76 | 0.73 | 0.0682 |
| TGAGG haplotype, homozygous | 0.44 | 0.78 | 0.73 | 0.0402 |
| random prediction | 0.26 | 0.74 | 0.50 | — |

This study showed that a specific genetic combination of at least two genes GCLM and GSS is strongly associated with the disease and can be used in combination with other criteria as predictors for disease status.

1. A method for diagnosis of a mental disorder comprising determining the level of expression of at least one gene involved in regulating the intracellular glutathione (GSH) level.

2. The method of claim 1, wherein the at least one gene involved in regulating the intracellular GSH level comprises glutamate-cysteine ligase (GCL), glutathione synthetase (GSS), glutathione peroxidase (GPX) and/or glutamate/cysteine exchange transporter (system Xc⁻) gene.

3. The method of claim 2, wherein GCL comprises the glutamate-cysteine ligase modulating subunit (GCLM).

4. The method of claim 2, wherein GPX comprises GPX1.

5. The method of claim 1 further comprising comparing the level of expression determined for a subject with the level of expression of the corresponding at least one gene for a subject or subject population not affected by the mental disorder; wherein a difference of more than 20 percent indicates that the subject is affected or at risk of being affected by the mental disorder.

6. The method of claim 1, wherein the level of expression is determined by measuring the level of transcription of the at least one gene.

7. The method of claim 1, wherein the level of transcription is determined by at least one oligonucleotide or polynucleotide able to bind to the transcription product of the at least one gene.

8. The method of claim 6, wherein the level of transcription is determined by techniques selected from the group of Northern blot analysis, reverse transcriptase PCR, real-time PCR, RNAse protection and microarray analysis.

9. The method of claim 1, wherein the level of expression is determined by measuring the level of protein expressed by the at least one gene.

10. The method of claim 9, wherein the level of protein is determined by antibodies, antibody derivatives, or antibody fragments.

11. The method of claim 10, wherein the level of protein is determined by Western blotting, FACS, immunohistochemistry, ELISA, ELISPOT utilizing an antibody, an antibody derivative, or an antibody fragment.

12. A method for diagnosis of a mental disorder comprising determining the level of activity of at least one protein involved in regulating the intracellular GSH level.

13. The method of claim 12, wherein the at least one protein comprises GCL, gamma-glutamyltransferase (GGT) and/or system Xc⁻.

14. The method of claim 12, wherein GCL comprises the glutamate-cysteine ligase catalytic subunit (GCLC).

15. The method of claim 12 further comprising comparing the level of activity determined for a subject with the level of activity of the corresponding at least one protein for a subject or subject population not affected by the mental disorder; wherein a difference of more than 10 percent indicates that the subject is affected or at risk of being affected by the mental disorder.

16. The method of claim 12, wherein the level of activity of system Xc⁻ is determined by measuring [³⁵S] cystine uptake.

17. A method for diagnosis of a mental disorder comprising determining the level of expression of at least one gene involved in regulating the intracellular GSH level and determining the level of activity of at least one protein involved in regulating the intracellular GSH level.

18. The method of claim 17, wherein at least one gene involved in regulating the intracellular GSH level comprises GCL, GCLM, GPX, GPX1, GSS and/or system Xc⁻ gene and wherein the at least one protein comprises GCL, GCLC, GGT and/or system Xc⁻.

19. The method of claim 17, which further comprises determining the level of intracellular GSH levels.

20. The method of claim 17, wherein the level of expression of GCLM, the level of activity of GCL and the intracellular level of GSH is determined.

21. The method of claim 20, wherein a decreased expression of GCLM and a negative correlation between GCL activity and GSH levels indicates that the subject is affected or at risk of being affected by the mental disorder.

22. The method of claim 12, wherein the level of activity of GCL is determined by measuring the amount of ¹⁴C-γ-glutamyl-aminobutyric acid.

23. A method for diagnosis of a mental disorder comprising determining the plasmatic level of at east one amino acid.

24. The method of claim 23, wherein the plasmatic level of cystine, glutamate, cysteine, homocysteine and/or cysteinyl-glycine is determined.

25. The method of claim 23 further comprising comparing the plasmatic level of cysteine and/or homocysteine of a subject with the levels of a subject or subject population not affected by the mental disorder; wherein a difference of more than 5 percent indicates that the subject is affected or at risk of being affected by the mental disorder.

26. A method for diagnosis of a mental disorder comprising determining the level of expression of at least one gene involved in regulating the intracellular GSH level and determining the plasmatic level of at least one amino acid.

27. The method of claim 26, wherein at least one gene involved in regulating the intracellular GSH level comprises GCL, GCLM, GPX, GPX1, GSS and/or system Xc⁻ gene, and wherein the at least one amino acid comprises cystine, glutamate, cysteine, homocysteine and/or cysteinyl-glycine.

28. The method of claim 26, wherein the level of expression is determined for GCLM and the plasmatic levels of cystine and glutamate are determined.

29. The method of claim 28, wherein a decreased expression of GCLM and an absence of correlation between cystine and glutamate levels indicates that a subject is affected or at risk of being affected by the mental disorder.

30. A method for diagnosis of a mental disorder comprising determining the level of activity of at least one protein involved in regulating the intracellular GSH level and determining the plasmatic level of at least one amino acid.

31. The method of claim 30, wherein the at least one protein comprises GCL, GCLC, GGT and/or system Xc⁻ and wherein the at least one amino acid comprises cystine, glutamate, cysteine, homocysteine and/or cysteinyl-glycine.

32. The method of claim 30, wherein the level of activity is determined for GGT and wherein the plasmatic level of cysteinyl-glycine is determined.

33. The method of claim 32, wherein an absence of a correlation of GGT activity and the level of cysteinyl-glycine indicates that a subject is affected or at risk of being affected by the mental disorder.

34. The method of claim 12, wherein the level of activity of GGT is determined by measuring the formation of 5-amino-2-nitrobenzoate.

35. The method of claim 23, wherein the level of amino acids is determined with an amino acid analyzer.

36. A method of claim 23, wherein the level of the at least one amino acid is determined by HPLC.

37. A method for diagnosis of a mental disorder comprising determining the level of expression of at least one gene involved in regulating the intracellular glutathione (GSH) level and determining the GSH level in blood.

38. The method of claim 37, wherein the level of expression is determined for GCLM.

39. The method of claim 38, wherein a decreased expression of GCLM and a decrease in GSH levels indicates that a subject is affected or at risk of being affected by the mental disorder.

40. The method of claim 1, wherein the method is performed ex vivo.

41. The method of claim 1, wherein the mental disorder is selected from the group of schizophrenic disorders, affective disorders, psychoactive substance use disorders, personality disorders, delirium, dementia, epilepsy, panic disorder, obsessive compulsive disorder, intermittent explosive disorder, impulse control disorder, psychosis, attention-deficit-hyperactivity disorder (ADHD), and manic or psychotic depression.

42. The method of claim 41, wherein the mental disorder is schizophrenia.

43. A composition for use in diagnosis of a mental disorder comprising at least one oligonucleotide or polynucleotide able to bind to a transcription product of at least one gene involved in regulating intracellular GSH level.

44. The composition of claim 43, wherein the at least one oligonucleotide or polynucleotide is able to bind to a transcription product of the GCL, GSS, GPX and/or system Xc⁻ gene.

45. The composition of claim 44, wherein GCL comprises GCLM.

46. The composition of claim 44, wherein GPX comprises GPX1.

47. The composition of claim 44, wherein the oligonucleotide or polynucleotide comprises at least one sequence selected from the group consisting of SEQ ID NO. 1 to 9.

48. The composition of claim 44, comprising the oligonucleotide of SEQ ID NO 3 and/or SEQ ID 4, and optionally SEQ ID NO 2 able to bind to GSS transcripts.

49. The composition of claim 44, comprising the oligonucleotide of SEQ ID No 6 and/or SEQ ID 7, and optionally SEQ ID NO 5 able to bind to GPX transcripts.

50. A composition for use in diagnosis of a mental disorder comprising at least one antibody, antibody derivative or antibody fragment able to bind at least one protein involved in regulating intracellular GSH level.

51. The composition of claim 50, wherein the antibody is a monoclonal antibody.

52. A composition for use in diagnosis of a mental disorder comprising at least one means able to determine the activity of at least one protein involved in regulating intracellular GSH level.

53. A composition of claim 51, wherein the at least one protein comprises GCL, GGT and/or system Xc⁻.

54. A composition for use in diagnosis of a mental disorder comprising at least one means able to determine the plasmatic level of at least one amino acid.

55. A composition of claim 54, wherein the at least one amino acid comprises cystine, glutamate, cysteine, homocysteine and/or cysteinyl-glycine.

56. A composition of claim 54, wherein the means comprise an amino acid analyzer.

57. The composition of claim 43 for use in diagnosis of schizophrenic disorders, affective disorders, psychoactive substance use disorders, personality disorders, delirium, dementia, epilepsy, panic disorder, obsessive compulsive disorder, intermittent explosive disorder, impulse control disorder, psychosis, attention-deficit-hyperactivity disorder (ADHD), or manic or psychotic depression.

58. The composition of claim 43 to 57 for use in diagnosis of schizophrenia.

59.-84. (canceled)

85. A method for prevention and/or treatment of a mental disorder comprising administering an effective amount of one or more proteins to a mammal including a human, wherein the one or more protein is selected from the group consisting of
a) GCL, GSS, GPX and system Xc⁻ or a fragment thereof
b) a bioactive protein having a percentage of identity of at least 50% with the amino acid sequence of any one of the proteins of (a);
c) a bioactive variant of any one of the proteins of (a) or (b).

86. The method of claim 85, wherein GCL comprises GCLM.

87. The method of claim 85, wherein GPX comprises GPX1.

88. A method for prevention and/or treatment of a mental disorder comprising administering an effective amount of one or more polynucleotides to a mammal including a human, wherein the one or more polynucleotide comprises a sequence encoding a protein as defined in claim 85, said sequence being operatively associated with a tissue specific or a constitutive promoter.

89. A method for prevention and/or treatment of a mental disorder comprising administering an effective amount of an agent that can alter the expression of at least one gene involved in regulating intracellular GSH level.

90. The method of claim 89, wherein the at least one gene involved in regulating the intracellular GSH level comprises GCL, GSS, GPX and/or system Xc⁻ gene.

91. The method of claim 90, wherein GCL comprises GCLM.

92. The method of claim 90, wherein GPX comprises GPX1.

93. A method for prevention and/or treatment of a mental disorder comprising administering an effective amount of an agent that can alter the activity of at least one protein involved in regulating intracellular GSH level.

94. The method of claim 93, wherein the at least one protein involved in regulating the intracellular GSH level comprises GCL, GGT and/or system Xc⁻.

95. The method of claim 94, wherein GCL comprises GCLC.

96. The method of claim, wherein GCL comprises GCLM.

97. A method for prevention and/or treatment of a mental disorder which comprises administering an effective amount of an agent that can alter the plasmatic level of at least one amino acid.

98. The method of claim 97, wherein the at least one amino acids comprises cystine, glutamate, cysteine, homocysteine and/or cysteinyl-glycine.

99. The method of claim 85, wherein the effective amount of the protein and/or the polynucleotide and/or the agent is administered orally, sublingually, intravenously, intramuscularly, intraarticularly, intraarterially, intramedullary, intrathecally, intraventricularly, intraoccularly, intrathecally, intracereberally, intracranially, respiratorally, intratracheally, nasopharyngeally, transdermally, intradermally, subcutaneously, intraperitoneally, intranasally, enterally, or topicaly, or via rectal means, infusion or implant.

100. The method of claim 85, wherein the mental disorder is selected from the group of schizophrenic disorders, affective disorders, psychoactive substance use disorders, personality disorders, delirium, dementia, epilepsy, panic disorder, obsessive compulsive disorder, intermittent explosive disorder, impulse control disorder, psychosis, attention-deficit-hyperactivity disorder (ADHD), and manic or psychotic depression.

101. The method of claim 85, wherein the mental disorder is schizophrenia.

102.-120. (canceled)

121. A method for the diagnosis of a mental disorder or a predisposition therefor in a mammal, particularly in a human being, comprising determining the presence of at least one polymorphism of at least one gene involved in regulating the intracellular glutathione (GSH) level and/or GSH-oxidative stress-related gene expression, wherein said at least one polymorphism is associated with said mental disorder or predisposition therefor.

122. The method of claim 121, wherein the at least one gene involved in regulating the intracellular GSH level and/or GSH-oxidative stress-related gene expression is selected from a glutamate-cysteine ligase, modifier subunit gene (GCLM) and/or a glutathione synthetase gene (GSS).

123. The method of claim 121, wherein said polymorphism is associated with low expression levels of at least one gene involved in regulating the intracellular glutathione level and/or GSH-oxidative stress-related gene expression.

124. The method of claim 121, wherein the polymorphism is located within an intron, the 3′ region and/or the 5′ region of the at least one gene.

125. The method of claim 121 comprising determining a single polymorphism in a chromosomal copy of the gene, wherein said polymorphism is associated with said mental disorder or predisposition therefor.

126. The method of claim 121 comprising determining a single polymorphism in two chromosomal copies of the gene, wherein said polymorphism is associated with said mental disorder or predisposition therefor.

127. The method of claim 121 comprising determining a combination of polymorphisms in a chromosomal copy of the gene, wherein said combination of polymorphisms is associated with said mental disorder or predisposition therefor.

128. The method of claim 121 comprising determining a combination of polymorphisms in two chromosomal copies of the gene, wherein said combination of polymorphisms is associated with said mental disorder or predisposition therefor.

129. The method of claim 121, comprising determining a combination of polymorphisms in at least one chromosomal copy of a combination of genes, wherein said combination of polymorphisms is associated with said mental disorder or predisposition therefor.

130. The method of claim 121, wherein the polymorphism of the GCLM gene is selected from the group consisting of
(a) the polymorphisms rs2235971, rs3170633, rs2064764, rs769211, rs718873, rs718875, rs2301022,
(b) polymorphisms being in linkage disequilibrium with at least one of the polymorphisms of (a), and
(c) combinations of polymorphisms of (a) and/or (b).

131. The method of claim 130, wherein the polymorphism is
(a) rs2235971, rs3170633, rs769211 and/or rs2301022,
(b) in linkage disequilibrium with at least one of the polymorphisms of (a), and
(c) selected from combinations of polymorphisms of (a) and/or (b).

132. The method of claim 131, wherein the polymorphism is
(a) rs3170633
(b) in linkage disequilibrium with the polymorphism of (a), and
(c) selected from combinations of polymorphisms of (a) and/or (b).

133. The method of claim 121, wherein the genotype of the polymorphism rs3170633 is selected from the group consisting of the nucleotides AA, AG and/or GG.

134. The method of claim 133, wherein the genotype is GG.

135. The method of claim 121, wherein a combination of polymorphisms in at least one chromosomal copy of the GCLM gene is selected from the group consisting of
(a) the polymorphisms rs2235971, rs3170633, rs769211 and rs2301022,
(b) polymorphisms being in linkage disequilibrium with at least one of the polymorphisms of (a), and
(c) combinations of polymorphisms of (a) and/or (b).

136. The method of claim 121, wherein the polymorphism of the GSS gene is selected from the group consisting of
(a) the polymorphisms rs3746450, rs725521, rs1801310, rs2236270, rs2236271, rs2273684, rs734111, rs2025096, rs3761144,
(b) polymorphisms being in linkage disequilibrium with at least one of the polymorphisms of (a), and
(c) combinations of polymorphisms of (a) and/or (b).

137. The method of claim 136, wherein the polymorphism is
(a) rs2236270, rs2273684, rs734111, rs2025096 and/or rs3761144,
(b) in linkage disequilibrium with at least one of the polymorphisms of (a), and
(c) selected from combinations of polymorphisms of (a) and/or (b).

138. The method of claim 137, wherein the polymorphism is
(a) rs3761144,
(b) in linkage disequilibrium with the polymorphism of (a), and
(c) selected from combinations of polymorphisms of (a) and/or (b).

139. The method of claim 121, wherein a combination of polymorphisms in at least one chromosomal copy of the GSS gene is selected from the group consisting of
(a) the polymorphisms rs2236270, rs2273684, rs734111, rs2025096 and rs3761144,
(b) polymorphisms being in linkage disequilibrium with at least one of the polymorphisms of (a), and
(c) combinations of polymorphisms of (a) and/or (b).

140. The method of claim 121, wherein a combination of polymorphisms in at least one chromosomal copy of a combination of the GSS gene and the GCLM gene is selected from the group consisting of
(a) the polymorphisms rs2235971, rs3170633, rs769211, rs2301022, rs2236270, rs2273684, rs734111, rs2025096 and/or 3761144,
(b) polymorphisms being in linkage disequilibrium with at least one of the polymorphisms of (a), and
(c) combinations of polymorphisms of (a) and/or (b).

141. The method of claim 121, wherein a combination of polymorphisms in two chromosomal copies of the GCLM gene or the GSS gene is homozygous.

142. The method of claim 121, wherein the polymorphism is determined by a genotyping analysis.

143. The method of claim 142, wherein the genotyping analysis comprises the use of polymorphism-specific primers.

144. The method of claim 142, wherein the genotyping analysis comprises a mass-spectrometric analysis.

145. The method of claim 142, wherein the genotyping analysis comprises a microarray analysis.

146. The method of claim 121, wherein the mental disorder is selected from the group of schizophrenic disorders, affective disorders, psychoaffective substance use disorders, personality disorders, delirium, dementia, epilepsy, panic disorder, obsessive compulsive disorder, intermittent explosive disorder, impulse control disorder, psychosis, attention-deficit-hyperactivity disorder (ADHD), and manic or psychotic depression.

147. The method of claim 146, wherein the mental disorder is schizophrenia.

148.-188. (canceled)


SEQUENCE LISTING (67 sequences)

SEQ ID NO: 1 - DNA, 25 bp, Artificial
  Primer/Probe
cacagcgaggagcttcatgattgta

SEQ ID NO: 2 - DNA, 16 bp, Artificial
  Primer/Probe
tgatggtgctggaaag

SEQ ID NO: 3 - DNA, 20 bp, Artificial
  Primer/Probe
ctgccttcctggagcaaact

SEQ ID NO: 4 - DNA, 22 bp, Artificial
  Primer/Probe
cgagcggtaaagtcatcctgtt

SEQ ID NO: 5 - DNA, 20 bp, Artificial
  Primer/Probe
tcttggcgttgtggtgatgc

SEQ ID NO: 6 - DNA, 18 bp, Artificial
  Primer/Probe
cccgtgcaaccagtttgg

SEQ ID NO: 7 - DNA, 24 bp, Artificial
  Primer/Probe
gacgtacttgagggaattctgaat

SEQ ID NO: 8 - DNA, 25 bp, Artificial
  Primer/Probe
cagctgcccttcctggacagcctat

SEQ ID NO: 9 - DNA, 25 bp, Artificial
  Primer/Probe
tatgctggctggttttacctcaact

SEQ ID NO: 10 - protein, 274 aa, Homo sapiens
MGTDSRAAKALLARARTLHLQTGNLLNWGRLRKKCPSTHSEELHDCIQKTLNEWSSQINPDLVREFPDVLECTVSHAVEKINPDEREEMKVSAKLFIVESNSSSSTRSAVDMACSVLGVAQLDSVIIASPPIEDGVNLSLEHLQPYWEELENLVQSKKIVAIGTSDLDKTQLEQLYQWAQVKPNSNQVNLASCCVMPPDLTAFAKQFDIQLLTHNDPKELLSEASFQEALQESIPDIQAHEWVPLWLLRYSVIVKSRGIIKSKGYILQAKRRGS

SEQ ID NO: 11 - protein, 474 aa, Homo sapiens
MATNWGSLLQDKQQLEELARQAVDRALAEGVLLRTSQEPTSSEVVSYAPFTLFPSLVPSALLEQAYAVQMDFNLLVDAVSQNAAFLEQTLSSTIKQDDFTARLFDIHKQVLKEGIAQTVFLGLNRSDYMFQRSADGSPALKQIEINTISASFGGLASRTPAVHRHVLSVLSKTKEAGKILSNNPSKGLALGIAKAWELYGSPNALVLLIAQEKERNIFDQRAIENELLARNIHVIRRTFEDISEKGSLDQDRRLFVDGQEIAVVYFRDGYMPRQYSLQNWEARLLLERSHAAKCPDIATQLAGTKKVQQELSRPGMLEMLLPGQPEAVARLRATFAGLYSLDVGEEGDQAIAEALAAPSRFVLKPQREGGGNNLYGEEMVQALKQLKDSEERASYILMEKIEPEPFENCLLRPGSPARVVQCISELGIFGVYVRQEKTLVMNKHVGHLLRTKAIEHADGGVAAGVAVLDNPYPV

SEQ ID NO: 12 - protein, 201 aa, Homo sapiens
  misc_feature (47)..(47): Xaa can be any naturally occurring amino acid
MCAARLAAAAAQSVYAFSARPLAGGEPVSLGSLRGKVLLIENVASLXGTTVRDYTQMNELQRRLGPRGLVVLGFPCNQFGHQENAKNEEILNSLKYVRPGGGFEPNFMLFEKCEVNGAGAHPLFAFLREALPAPSDDATALMTDPKLITWSPVCRNDVAWNFEKFLVGPDGVPLRRYSRRFQTIDIEPDIEALLSQGPSCA

SEQ ID NO: 13 - protein, 501 aa, Homo sapiens
MVRKPVVSTISKGGYLQGNVNGRLPSLGNKEPPGQEKVQLKRKVTLLRGVSIIIGTIIGAGIFISPKGVLQNTGSVGMSLTIWTVCGVLSLFGALSYAELGTTIKKSGGHYTYILEVFGPLPAFVRVWVELLIIRPAATAVISLAFGRYILEPFFIQCEIPELAIKLITAVGITVVMVLNSMSVSWSARIQIFLTFCKLTAILIIIVPGVMQLIKGQTQNFKDAFSGRDSSITRLPLAFYYGMYAYAGWFYLNFVTEEVENPEKTIPLAICISMAIVTIGYVLTNVAYFTTINAEELLLSNAVAVTFSERLLGNFSLAVPIFVALSCFGSMNGGVFAVSRLFYVASREGHLPEILSMIHVRKHTPLPAVIVLHPLTMIMLFSGDLDSLLNFLSFARWLFIGLAVAGLIYLRYKCPDMHRPFKVPLFIPALFSFTCLFMVALSLYSDPFSTGIGFVITLTGVPAYYLFIIWDKKPRWFRIMSEKITRTLQIILEVVPEEDKL

SEQ ID NO: 14 - DNA, 1610 bp, Homo sapiens
ggcacgaggctgcggccgcagtagccggagccggagccgcagccaccggtgccttcctttcccgccgccgcccagccgccgtccggcctccctcgggcccgagcgcagaccaggctccagccgcgcggcgccggcagcctcgcgctccctctcgggtctctctcgggcctcgggcaccgcgtcctgtgggcggccgcctgcctgcccgcccgcccgcagccccttgcctgccggcccctgggcggcccgtgccatgggcaccgacagccgcgcggccaaggcgctcctggcgcgggcccgcaccctgcacctgcagacggggaacctgctgaactggggccgcctgcggaagaagtgcccgtccacgcacagcgaggagcttcatgattgtatccaaaaaaccttgaatgaatggagttcccaaatcaacccagatttggtcagggagtttccagatgtcttggaatgcactgtatctcatgcagtagaaaagataaatcctgatgaaagagaagaaatgaaagtttctgcaaaactgttcattgtagaatcaaactcttcatcatcaactagaagtgcagttgacatggcctgttcagtccttggagttgcacagctggattctgtgatcattgcttcacctcctattgaagatggagttaatctttccttggagcatttacagccttactgggaggaattagaaaacttagttcagagcaaaaagattgttgccataggtacctctgatctagacaaaacacagttggaacagctgtatcagtgggcacaggtaaaaccaaatagtaaccaagttaatcttgcctcctgctgtgtgatgccaccagatttgactgcatttgctaaacaatttgacatacagctgttgactcacaatgatccaaaagaactgctttctgaagcaagtttccaagaagctcttcaggaaagcattcctgacattcaagcgcacgagtgggtgccgctgtggctactgcggtattcggtcattgtgaaaagtagaggaattatcaaatcaaaaggctacattttacaagctaaaagaaggggttcttaactgacttaggagcataacttacctgtaatttccttcaatatgagagaaaattgagatgtgtaaaatctagttactgcctgtaaatggtgtcattgaggcagatattctttcgtcatatttgacagtatgttgtctgtcaagttttaaatacttatcttgcctccatatcaatccattctcatgaacctctgtattgctttccttaaactattgttttctaattgaaattgtctataaagaaaatacttgcaatatatttttcctttatttttatgactaatataaatcaagaaaatttgttgttagatatattttggcctaggtatcagggtaatgtatatacatattttttatttccaaaaaaaattcattaattgcttcttaactcttattataaccaagcaatttaattacaattgttaaaactgaaatactggaagaagatatttttcctgtcattgatgagatatatcagagtaactggagtagctgggatttactagtagtgtaaataaaattcactcttcaatac

SEQ ID NO: 15 - DNA, 1918 bp, Homo sapiens
gaggccccgccccctgagcctgggtagcggcgcgagggccgggagaaccgttcgcggaggaaaggcgaactagtgttgggatggccaccaactgggggagcctcttgcaggataaacagcagctagaggagctggcacggcaggccgtggaccgggccctggctgagggagtattgctgaggacctcacaggagcccacttcctcggaggtggtgagctatgccccattcacgctcttcccctcactggtccccagtgccctgctggagcaagcctatgctgtgcagatggacttcaacctgctagtggatgctgtcagccagaacgctgccttcctggagcaaactctttccagcaccatcaaacaggatgactttaccgctcgtctctttgacatccacaagcaagtcctaaaagagggcattgcccagactgtgttcctgggcctgaatcgctcagactacatgttccagcgcagcgcagatggctccccagccctgaaacagatcgaaatcaacaccatctctgccagctttgggggcctggcctcccggaccccagctgtgcaccgacatgttctcagtgtcctgagtaagaccaaagaagctggcaagatcctctctaataatcccagcaagggactggccctgggaattgccaaagcctgggagctctacggctcacccaatgctctggtgctactgattgctcaagagaaggaaagaaacatatttgaccagcgtgccatagagaatgagctactggccaggaacatccatgtgatccgacgaacatttgaagatatctctgaaaaggggtctctggaccaagaccgaaggctgtttgtggatggccaggaaattgctgtggtttacttccgggatggctacatgcctcgtcagtacagtctacagaattgggaagcacgtctactgctggagaggtcacatgctgccaagtgcccagacattgccacccagctggctgggactaagaaggtgcagcaggagctaagcaggccgggcatgctggagatgttgctccctggccagcctgaggctgtggcccgcctccgcgccacctttgctggcctctactcactggatgtgggtgaagaaggggaccaggccatcgccgaggcccttgctgcccctagccggtttgtgctaaagccccagagagagggtggaggtaacaacctatatggggaggaaatggtacaggccctgaaacagctgaaggacagtgaggagagggcctcctacatcctcatggagaagatcgaacctgagccttttgagaattgcctgctacggcctggcagccctgcccgagtggtccagtgcatttcagagctgggcatctttggggtctatgtcaggcaggaaaagacactcgtgatgaacaagcacgtggggcatctacttcgaaccaaagccatcgagcatgcagatggtggtgtggcagcgggagtggcagtcctggacaacccataccctgtgtgagggcacaaccaggccacgggaccttctatcctctgtatttgtcattcctctcctagccctcctgaggggtatcctcctaaagacctccaaagtttttatggaagggtaaatactggtaccttcccccagctttccatctgaggaccagaaaagttgtgtctcccttagatgagatctagacgcccccaaatccttgagatgtgggtatagctcagggtaagctgctctgaggtaaaggtccatgaaccctgccccactcctgtcagcccctcatcagccttttcagcaggttccagtgcctgacttgggataggactgagtggtaggaggagggggagtggaggggcatagcctttccctaattctgccttaaataaaactgcattgctgattcaaaaaaaaaaaaaaaaaaa

SEQ ID NO: 16 - DNA, 1134 bp, Homo sapiens
cgacccctcgaggggcccagccttggaagggtaactggaccgctgccgcctggttgcctgggccagaccagacatgcctgctgctccttccggcttaggaggagcacgcgtcccgctcgggcgcactctccagccttttcctggctgaggaggggccgagcctccggtagggcgggggccggatgaggcgggacctcaggcccggaaaactgcctgtgccacgtgacccgccgccggccagttaaaaggaggcgcctgctggcctccccttacagtgcttgttcggggcgctccgctggcttcttggacaattgcgccatgtgtgctgctcggctagcggcggcggcggcccagtcggtgtatgccttctcggcgcgcccgttggccggcggggagcctgtgagcctgggctccctgcggggcaaggtactacttatcgagaatgtggcgtccctctgaggcaccacggtccgggactacacccagatgaacgagctgcagcggcgcctcggaccccggggcctggtggtgctcggcttcccgtgcaaccagtttgggcatcaggagaacgccaagaacgaagagattctgaattccctcaagtacgtccggcctggtggtgggttcgagcccaacttcatgctcttcgagaagtgcgaggtgaacggtgcgggggcgcaccctctcttcgccttcctgcgggaggccctgccagctcccagcgacgacgccaccgcgcttatgaccgaccccaagctcatcacctggtctccggtgtgtcgcaacgatgttgcctggaactttgagaagttcctggtgggccctgacggtgtgcccctacgcaggtacagccgccgcttccagaccattgacatcgagcctgacatcgaagccctgctgtctcaagggcccagctgtgcctagggcgcccctcctaccccggctgcttggcagttgcagtgctgctgtctcgggggggttttcatctatgagggtgtttcctctaaacctacgagggaggaacaccttgatcttacagaaaataccacctcgagatgggtgctggtcctgttgatcccagtctctgccagaccaaggcgagtttccccactaataaagtgccgggtgtcagca

SEQ ID NO: 17 - DNA, 3144 bp, Homo sapiens
atggtcagaaagcctgttgtgtccaccatctccaaaggaggttacctgcagggaaatgttaacgggaggctgccttccctgggcaacaaggagccacctgggcaggagaaagtgcagctgaagaggaaagtcactttactgaggggagtctccattatcattggcaccatcattggagcaggaatcttcatctctcctaagggcgtgctccagaacacgggcagcgtgggcatgtctctgaccatctggacggtgtgtggggtcctgtcactatttggagctttgtcttatgctgaattgggaacaactataaagaaatctggaggtcattacacatatattttggaagtctttggtccattaccagcttttgtacgagtctgggtggaactcctcataatacgccctgcagctactgctgtgatatccctggcatttggacgctacattctggaaccattttttattcaatgtgaaatccctgaacttgcgatcaagctcattacagctgtgggcataactgtagtgatggtcctaaatagcatgagtgtcagctggagcgcccggatccagattttcttaaccttttgcaagctcacagcaattctgataattatagtccctggagttatgcagctaattaaaggtcaaacgcagaactttaaagacgcgttttcaggaagagattcaagtattacgcggttgccactggctttttattatggaatgtatgcatatgctggctggttttacctcaactttgttactgaagaagtagaaaaccctgaaaaaaccattccccttgcaatatgtatatccatggccattgtcaccattggctatgtgctgacaaatgtggcctactttacgaccattaatgctgaggagctgctgctttcaaatgcagtggcagtgaccttttctgagcggctactgggaaatttctcattagcagttccgatctttgttgccctctcctgctttggctccatgaacggtggtgtgtttgctgtctccaggttattctatgttgcgtctcgagagggtcaccttccagaaatcctctccatgattcatgtccgcaagcacactcctctaccagctgttattgttttgcaccctttgacaatgataatgctcttctctggagacctcgacagtcttttgaatttcctcagttttgccaggtggctttttattgggctggcagttgctgggctgatttatcttcgatacaaatgcccagatatgcatcgtcctttcaaggtgccactgttcatcccagctttgttttccttcacatgcctcttcatggttgccctttccctctattcggacccatttagtacagggattggcttcgtcatcactctgactggagtccctgcgtattatctctttattatatgggacaagaaacccaggtggtttagaataatgtcagagaaaataaccagaacattacaaataatactggaagttgtaccagaagaagataagttatgaactaatggacttgagatcttggcaatctgcccaaggggagacacaaaatagggatttttacttcattttctgaaagtctagagaattacaactttggtgataaacaaaaggagtcagttatttttattcatatattttagcatattcgaactaatttctaagaaatttagttataactctatgtagttatagaaagtgaatatgcagttattctatgagtcgcacaattcttgagtctctgatacctacctattggggttaggagaaaagactagacaattactatgtggtcattctctacaacatatgttagcacggcaaagaaccttcaaattgaagactgagatttttctgtatatatgggttttgtaaagatggttttacacactacagatgtctatactgtgaaaagtgttttcaattctgaaaaaaagcatacatcatgattatggcaaagaggagagaaagaaatttattttacattgacattgcattgcttccccttagataccaatttagataacaaacactcatgctttaatggattatacccagagcactttgaacaaaggtcagtggggattgttgaatacattaaagaagagtttctaggggctactgtttatgagacacatccaggagttatgtttaagtaaaaatccttgagaatttattatgtcagatgttttttcattcattatcaggaagttttagttatctgtcatttttttttttcacatcagtttgatcaggaaagtgtataacacatcttagagcaagagttagtttggtattaaatcctcattagaacaaccacctgtttcactaataacttacccctgatgagtctatctaaacatatgcattttaagccttcaaattacattatcaacatgagagaaataaccaacaaagaagatgttcaaaataatagtcccatatctgtaatcatatctacatgcaatgttagtaattctgaagttttttaaatttatggctatttttacacgatgatgaattttgacagtttgtgcattttctttatacattttatattcttctgttaaaatatctcttcagatgaaactgtccagattaattaggaaaaggcatatattaacataaaaattgcaaaagaaatgtcgctgtaaataagatttacaactgatgtttctagaaaatttccacttctatatctaggctttgtcagtaatttccacaccttaattatcattcaacttgcaaaagagacaactgataagaagaaaattgaaatgagaatctgtggataagtgtttgtgttcagaagatgttgttttgccagtattagaaaatactgtgagccgggcatggtggcttacatctgtaatcccagcactttgggaggctgagggggtggatcacctgaggtcgggagttctagaccagcctgaccaacatggagaaaccccatctctactaaaaatacaaaattagctgggcatggtggcacatgctggtaatctcagctattgaggaggctgaggcaggagaattgcttgaacccgggaggcggaggttgcagtgagccaagattgcaccactgtactccagcctgggtgacaaagtcagactccatctccaaaaaaaaaaaaaaaa

SEQ ID NO: 18 - DNA, 23353 bp, Homo sapiens
atcaaaatgtgtttaagtagtggtggtgcttgtgggtagctctttcactataataaaaaatcaatatccaaaaaaaaaaaaaaaaagcagcgcaagctcgttcaatgaactcttaccatcattcattcagccaacaaatatttactcaacatcaggctcaatttctaacttcctagaaagatcttttttcccatccctcccctaattcttaccattttaccaactggttcatctctcttttcaatagctatattgtccccgcttataatgagaatctcattttgcctttaataatctgattcacctcaacgaatgtgaaagattgttgaggaaagctcaatacatgttcaatttgatgtttataccgagtccaccaccacacgtgaaatttaataggggtaaataatctcgaccacctctaaaggggtgggtgataaaagctttctgtgatctcaccggctcaagtgtcctctttaatctgaattcatctggagaataactgccctatgatgttcttataaaattacttgtttcctgtgcttagaacttgtctctgggtagagtgtacgcgtctcaagggcaaagactcagtcttgttcgttgacttcacacactgccagcaaataatgtgaatcaatagacctttaacggatgaagaaaaattagcagtttgggagaaggtccttgaaggtagcaaagcgaaaagaagaagggggcgcttgcgcataaacagctgcttctgagaacgaaaactacgtagcatttccgagtcccggtggccgctcaggctgccctttaaagagacgtgtaggaagcccaccctggggcggccgtgggcggagccgcgacctgaacgccgggagacctcaccaggcggaggcggtgtctacctcaccaggcggaggcggtgtctgcctcgcgctgtcctctgaccgccagggggagccctgccgcgcgctgcgctccgggccgcgccgccacgctctctcgacccgcgcgcccgccgcgcaccacccgtcgccacgcccgccgcaggccaagggccagtcacttgcgggccggcgtcccgcagcccattcgcgccccgcccctgccccgccgcgggatgagtaacggttacgaagcactttctcggctacgatttctgcttagtcattgtcttccaggaaacagctccctcagtttggaatcagctctcccgctgcggccgcagtagccggagccggagccgcagccaccggtgccttcctttcccgccgccgcccagccgccgtccggcctccctcgggcccgagcgcagaccaggctccagccgcgcggcgccggcagcctcgcgctccctctcgggtctctctcgggcctcgggcaccgcgtcctgtggggcggccgcctgcctgcccgcccgcccgcagccccttcgctgcgcggcccctgggcggccgctgccatgggcaccgacagccgcgcggccaaggcgctcctggcgcgggcccgcaccctgcacctgcagacggggaacctgctgaactggggccgcctgcggaagaagtgcccgtccacgcacagcgaggaggtgagcgtggcgacactcgccgcggggctcccggggcaggcctcgggcggggccgggcggttccggcaagggagaagcgcccgcgggcgagggagggtggcagggcctcgcgcgcacctgggctccgcccgcggtcagatgctgaggccagtgtcgacgcgaccttgggtccaaacccgctgccgagggcaacgcgtctaaaaaacttagccggtgcagctgcgcactgagtgacacctctctcggtgcttaaggctggtagcaaaactctcaaacatcctgggtccctcttcctggcacggaggtcacagagggccaggacgtcgctccgtctctctccacatctctcgcttccttcccccaccctgtccaggtaaaggaatattttcggagattatcctttggtgcattcagatgaagtagtattcatagtacacctaacttttagaatgttgacattagactctctattgcagtgaggcaaatgagtaagcactcaattttggctttggaataccctaaataccctgactttagtcattacacattttatgcacttatctgtgcgtctatgtatgctttatcagtacacattctatgcatgtaacaatatcattgtacattctatgcatgtaacagaatatcacatgtgccccataaatatgtacaaatataatctatcaattcaaaaattaaagttttgtaaaaactggaaaataaaaagactagtcactagttggaggtgacagagattaatctggaattacaaacttgttacctatttgttggccatttctgagcggatagtcatccactgcacattgtgcatattggtggtatgggacaattttagtatttaaaaaataacaaaatctttagatttaggagctatatgactttggaaaattactttaaagttggataataacaccttcctcatcaggttgttgtgagaattaagaggtaatatatgaaaacgctcaccatgcaggcatctatctccctgtcatctggttccatcagaaagataaatcttaattaagagaaaaaccttcctaaactatgtgtgtttagaaaaaccttcctaaatcagctgtgttcctaagtggtttctgctctcttctatgagatgttggtaacagaccatgcagcagcacacggcatccctctgagttaagtgctggacctgtgcccaggcctgttgttggggtcagtgcccatcaagagatgagaactcttgcttgtaactttagtaggaatctgagtctgtaccatattctgtgttcacgttccagctcagtgtgtatgttttgttttatttaaaccacctactttggctgtttctcccacagataaatatttctgtattcggtacatagactcttaatttcttccagtactgttggtggtagttcaatacagaattttgcacgtatattgatattattgggtgaaattttacctatctcacaatagcatactctaaagtagtgactcaaaagagtttctgactgcaaacacagtaagaagtacattttactttgcaatccagttaatagttgtataatcagtatatctgaaacaggtttcacaaaaagatattagcccttataccgtatattctgattttttcttttattttaatttttaaaatgttgatttcttgatgctcagagtcacacaccacagtttgtaaaacattgttcaaaggactaattctggtttctagtgttttatttagggtgcttcataacagtaggataaaactaaagaaactagaaaacatttcaggaaaacttgaaggaatggcatcatactgtagggaaactatgactgcttggtaagagcaaaaggggaggcacattttgtttctatgtaaaagaactttgtaacacccagagctccaaattgggatgtactacttggaacagttccgcttcactgaagaaattaagtaaactgaacaaatcacttatttttgagaaaatgtacgtgttgggtcaagagttcagttaaatttcttacaaggtctcttctagttctaattttttaaaaaatgttatgacctctgcccagattttttgtctcactggaattttatgaaatcaaatagtttgtaagtggaccattataggactgttttgcccagttctttgttgtaagggtgtttgaccggttgaatcatggtatttaaaaaattcttatacaactccagatctaatggtaggctaagttgtggtgatgcttatactcagtgatattgggtgtgtattataagaatgaagagagcggagaacaaacataaacattaatgttaatgacaaacattaacccaagtacaaggttaatgtttagtcaatatagcaaacatgtaatttacaagattaaaaataattaggcttgtgataaagtcaatgaatttcctacgtaattgtaacattagactgttttattatttgtcctgacattttgcagaatccaagattaattaaagaaatggtttcaagaagagggtgaatactataaaaatagacttaccttcctgaattgaggaattcatcaggaaagcctcaagtgtgcaaatgagccatccttccagagggaaatttcttagaattatcccacgatttgagccaaagcacttccgatagaatttttaacctctagttggttctgctccttccatttttactaatttttaagaaaatactatgacttataattgtatctggaatgattatcaactccttttcatccactgacttaaatttgattataaatatgctttacataaagatctagaccttataatttgaattcaagtgaattgttgtgactagcatgtaaattattattatggattgtaaatcttaacataggtagttctgtgcccttaaattgataaaccagttatctcttgtaatcatgtgtactaagatatacgtagtaaagtgattgtatcagtttttatcataagcagtcatagttcagatagttcagaagtttagtgtctgctgtttctattaggaaagtgcttttgcaattcaggggcttgcctctagttactcatttgtgttttcattgattgcccacagaagaatgcaaaacactgtgtccttggttatctaataaggcagtgatttcagattgccaggtttggcccattagtggtcataaatcagttcagtgagtcatggaccagtatattttttctaattaatatattggaagccattagagtgcctggcatgttgtgagcataaataccgctttatgactgttttgttccattatatgtatataggtctttgctcactatgtgaatgtatgtcttactttggactactatcaaataagtctgaaatacactttaataaagtatgtttgaattaatttacaacatactataatatttgtataaatttttttttgtttatctaagaaaacatggaagataggttagaggtgcacctaaatgttacgaaatgacttctggagtgtcatacaagttgagttctgcaagatatgtctatcctggtggttctcagactttagagtacgtagataaagtactctccttaggatgcttgttaaaatgaaagtccaaactgctcttggagaatgattcagtgggtctaggattaggctcaggagtctgaaattttaataaacagcacaggtgaatctgaggtaggatttaccagccacacttattctctaataatttaatttgtttgtctttgagaaatgtagcatagttgtgatatgaattctagtcagacagcctgggcttgactccaagttcctttgtttataaaccttatttttgtttctttatgaatgcctttttttttttttttttttggaaatagagtcttgctctgttgcccaggctggaatgaaatggcatcatctcagcacactataacctccacctcctgggttcaagcaaaattgtcctgcctcagccacccgagtagctactacaggcttgcactactaggcccacatgatttttgtatttttagtagagacagggtttcaccatgttggccaggctggtcttgaactcctgacctcaagtgatttgcccccctcagcctcccaaagtgctgggattacaggcatgagccactatacctggcctatgaatgcttttgtgtctcaacagttagcttaaattttcactggtacaaaaaaagctttaaagtaattagacaacttctttagttcaagaacatttgagaaacaagagtaggatgaaaacaggcctgaatttatgtatgttgaaggaaacaaaaaccttgtaggattaaaattggggtttgtatggtatttcctgttgctatcttttttttcctgcttatcaaattaatatattcttattgtagaagtacagaaaagtatatagactttgattaacagtttcaaaatgcattccttttgcagcttcatgattgtatccaaaaaaccttgaatgaatggagttcccaaatcaacccagatttggtcagggtaagtgaaatgtgaatgcaaaaatcatgcaggtcaaaaaggaagtaaagtaaacaggaagttaatttcttcagttttgtgagggaagtcaaataggcaattagccataaactctctgtggttgatttgctgagacggcactctgatccagtaggcttatgcataaaacagaaaatctagaccgtaggcaactctttaaccagacaagatactaggattctcatgcttaattctcaccatggtttcctcactatcagtgttttatttcagtttatatttggtctaggagaggaatgattatcaattgtgattggtagattgctttagtagactcaacatcactagaagtaatttttcaaatgtcagtttctgatgaaacaataaggaactgtgttccactaaatgtcagtatatggctactatcataaatgttaatgttcaaaaccctaaaacacattttgaaatccaactcagtcaaaggctcacagcacattacttaggctactttaaaagtatggaaaaggacatgtgctggaaatactggttcccctgggcatactgcaaccatgtaactctatagctactatatagacattgagttatggttttttttaaatcaatctctgtttctctgaaatgactgtttattctacttatgcttgacttgtaaacacttactgaacccctgatacgtgctgtgaaagtgctcaagaatcatgggaaagcctttgccgtttactttgtatgatattgtaaatgtaagttaatatgtatctgatttatatgtactaatattttctcattatccttgtaaattattcattaaaatacattcaaatagcctttgcttttttctgctgcactcaggaaaaaaaaagttttggtttacaatgcttgtgattacaggctgggcgcagtggctcacgcctataatcccagcactttgggaggctaaagcaggtagatcacctgaggtcaggagtttgagaccagtctggccaacatggcgaaaccccatgtctactaaaaatacaaaaaaaaaaaaaaagccaggcgtggtggtacccgcctgtaatcccagctactcgggaggctgaggcaggagaatcacttgaacctgggaggcagaggttgcagtgagctgagatcacaccactgcactctagcctgagcaagagagtgagactctgtctcaaaaaaataataataataaaattaaataaataaataaataagaataaaatgcttgtgattataaaactaaatatactattgtcataaaatcagcaattatgctccttagtatctacccaaagaagctaagaacttatttccacacacagacctatgcatggatatttatagcagctccattcataattgctaaaatttgaagcaaccaagatgttcttcagtaggcgagtgtatatataaattatgaatgcatatataaactatccagacaatataatattattcagtgcttaaaagaaatgagctatcaagccatgaaaagacatagaggaaacttgaatgcatatttctaagtaaaagaagccaatctgaaaaagctacataatgtatgatatcaactatttgatattctggaaaaaacaaaactatgtagactatagttttgctagtctagtaaaaaaaactagtaaaaagatcagttgttgccaggggttagcagggacggagggatgaataggcagagcacaggatttgtatgtcaatgaaactactgcatatgatattgtaatggtggatacatgttattatatggttttctaaatccataaactatgcagcaccaagagtaaaccctaagataaactggacttgggtgattatgacatgtcaatgtaggttcatcaagtgtaacaaatgtataccactgtggtgggtatgttgataatgggggaggctgtgtggatgtgagtgtagggagtgaatgagaaatctgttcaatctaccttctacttgatattcttgtgaacctaaaactgctctaaaaaaaaagtatatttaaaaaaaaaaaaacaggctgggcgcggtggctcatgcctgtaatcccagcactttgggaggccgaggtgggcggatcacaaggtcaggagatcgagaccatcctggctaacacggtgaaaccctgtctctactaaaaatacaaaaaattagtcaggcgtggcggcgtgcacctgtagtcccagctactcgggacactgaggcaggaggaatggcgtgaacccgggaggcagagcttgcagtgagccgagatcgtgccactgcactccagcctgggcgacagagcgagactctgtctcaaaacaaaacaaaacaaaacaaacaaacaaaaaaacccaaaaactaaatccccttacaggttaaagttcaaataacattaaaatccaaataaattaaaaagtaaaagaaaatgcttgtaattaatgtagaacgtgtttattatgtcatctagagtggatattattttcattggtttctttttagagtaaaattatctgctgtgctactgtttctaactcctcacatatccctccactgcttgtttatttttaatttttttgatcatgcttctttctaaatacctggtcaaaatgagatacattttcctagaaaatagatacacacacacaaacacacacacacaccccactttacctttatttcagagctttacaacccttgtgaagcccacccagacctcaggtagagaactctgttgtcttataggttctctgattctaaaaggcattatggataatagaaaattttataattaatcagaagcattaaattttatatttgttttaaatattgtgtttattataggagtttccagatgtcttggaatgcactgtatctcatgcagtagaaaagataaatcctgatgaaagagaagaaatgaaagtttctggtaaagtccagtcttttctgtttattttgttacatagcggaagcgatagattttgttaggcattagaataagcctttctttgctcagacactaaatgtgggaaatacagaaattcttttttagcataattttacttaaactccaagatggagttataagaggtcagtgctggctattatataagagtcagaatagaacttcaacttgctgttcttcatgatgatgtcatcatgtcatgtaatttatgtgtcaaacttcagagcagtagaaagattaagatgaaggagatggaaaggataaagggaaggcttagaagcagcatccacccaaagcaagcataagcacccccttcctggtaagatactggtaagcaatgtgtgaggtcacctgggaatataaaaagaaacctgggcaagttacatgaagagaaagaaaatactgtaacacccattcaggaaatgtagtaagtgacaaattccagaaactgtactatactcaaggaatacaaaaatgaaaagatgcgccatctctgccctcaccttacaattagcatagcatagtcattaaaagcttgagctttgaagtttcggggtgtgcagattacaggctagtataatgttgggctaacaaagactttaacctccctgagtctcagttttctcttctataaaatggggatgtacctgtttctccggcttctttgtaaactggatgaaataacaaaagaagcacttagcacagtgtttgcctcaaagtaagttctaggtagctattatttgctaccatgttgttattattgtggttgttgctattttattattattgagatggataataataataaattacatgtaagatgagatagggtattgtctctttttaatctaattatagtaactaccatttattgagtcttgctttgtgccaggcactgtgcttggtactttatggttatattttctcatttaattctcaagacaatcctgcaagttaagtctgttactgcattgcattagtgatactaaataacttgaaggttgtcatacaaccagtaagtggttgaaccaggaatccactcaggtatgcctaactccaaaacccattttttcctgttaatttcacattgctgtttgtgttggaaaggacgtaaagcaacattctttctgaggttgactgtaattcatgatttagttccatcttgggtctcttagaagtcctcagttcctagagggcttgagatttttttataggtatcaaaattctccccatataaagagctttcctctttctacctatccaaaaaagcctcatccttcagagagaagaggccatttcagatggcacaaattcatggacagagaaatagtattggggggggctttcatagagttataggggctaaagttagaccaaaatagtaaatactgactctcatctagtagagaagctaggatggctgttcaatagggagtaatctcagggaagcagtgtttttagggagatcacctagcagtaggaaggaagggactcatataagaaacgtagaagaaaatgtgttgcatatatcgaatgtcagcactttatgtaagggtatttcattagccttatttttctccatttcctcttttgattttttttgttgctcttctgctactctacctgtttgtctattgaatacatttatttttttgaacagtgagtttacataaagtaaatatgtacatataaagttgaatttgaatgaaaccaaaacctcaatattacaaatatagaaaatgactcaactttcctagtaaccaaagaaatgacaattaatgtggtcttgggtaggtatgcattgtacctactaagttttaaaaaaaaattaataatagcatcctatgttgttagaagagcaattgttaattggtggtggtgtggtaaactggcacacatcttttaaaatataataagactcacacaaaacgtcacatgctttgactcataataacatttctaacaattcatccctgggaaattatatagtataatgctgaataaaaagttactaagatgtttgttttggcattatatatgcgaaaacagaaaactggaactagtactggaacatttactaacctaaatgttcaataagtagaactcatttaataaatgacactcttcaaaatttacaaaataatattcaaccattaaaaaatgagcatgtaaaattggctgggcgcagtggctcacgcctgtaatcccagcactttgggaggcgagtcgggtggatcacttgaggccaggagttcgagaccagcctggacaacatggcaaaaccccgtctctactaaaaatacaaaaattagccaggcatggtggcgcaggcctataatcccagctacttgggtggctgaggcatgagaattgcttgaacccaggaggaggttgcaatgaaccaagatggcaccactgggcgacagagtgagactctgtctcaaaaaaaaaaagtatgtaaaattatacactaaaaacatagaatacgttcaaggccacccctaagtggaaaaaaagccacagaatacacagaaatctaaacagtaatttgggtgatgagactggcaattctttttggtctttctgcttttataggtcttcgttatttctacagtaagcacatgactattaggataaaaaaaatcttggcctggtgtggtgctgtaatcctagcaccttgggaagattgtttgagaccgggagttcaagagcccctgggcaacatatcgagaccctgtgtctacaaaaaaatgaaaattagccaggtgtattggtacatgcctgtagtcctagctacttgggaggctgaggtgaaaggaacacttgagctcagaaggttgatgctgcagtgagccgtgatcgcaccactgcactccagcctggatgaaagtgagaccctgtctcaaaaaaaaaaattgtggtgattcacacctgtaatcacagcactttgggaagccgaagcgggagggtcctttgaggccaagagttcaaggccagcctgggcagtataatgagaccctgtctctacaaaaaatttttaaaagtaaagaaattttaagataactaaatactacatagacatatattttaaatatttattacataaaggtaaaccaaatagaagaggaaataatgttatgccctacttcatatgaccaaaaactggaagatagtgtctgaaaatgaaaatgattgtattgggaaggtagaattgtggccttttttttttttttttttttttttttcagttttcttctcattacattttcaatttagtctttgtatatagattttggtttattggagaatatataatgtgctctattaatgtttaagtcataaaaatataaatttcaagtaatttaagctccaatagttatctaacctgccttctaataaatgggaaataaatatttactttttgttttgataaacatatatttgttggcaactagcacatgattttaaaagtatagtggaactatacatttatgtcttaaaattaaaactataaagttatgtgactgggaaaggaaaaataattcattcaggattatctgacatcttagtattatagtagtgttaatactagcatatagtgaaatgtgtatccaaatgtagtaatcagttttgttttctttgttacccagcaaaactgttcattgtagaatcaaactcttcatcatcaactagaagtgcagttgacatgggtaagcaaaaactgaattttttatcttaatagtggactttaaattagtataggtgtattagttataacttgtgcttaggtccaggtaaaaagaaaatgagttgattccaattttaccttttaaagttctagcttagtttcttaaggcttctgtaaaatcatactgacttatgtagggtcatattttgatatgcaagaggaatgtaaaaggtgtaatgtacttaaatgttcactatccgtttggagatagaagttcttattcttagcccttttagtactagaggtgaaatatcaaaaccaaacaacggcaaaaaaaaaaaaatccaaaaaaaatccaagtatatctgtttttagcacttaggttattgtgatattaaagttattcaatacttaaataaaaaattattccagtatttacatatattgaaaacttaaataattggctctttaatgtaactttagaatgaataatcatttaaaataagctcttaaattatttttatagaattaagaaattatctacctctataaatatggattaagaccaaacattcagtttttcagtgtctcaagcttattctttgctgtgactactagttacagaatattaggtcataattcttgtgatcatccatatgcgatagtagtgagcaagtagaactggagaagaaaattttatgaagataagggagctactaaaaacttctgggacctaggaaacaagaagaaattttgttccctagaaagaggagcatgtagtagtagctgcaagctgcctaggaataaggcaggtcagagaaacccacttgtctgccacacagagcaagtaggtaaatgggcacttacacatttttgaaagatagaaatgttctaaatgaaaagtaaactatgaaataatctgttattttgaaaacaagtgaagaatatggacagaattcagtgggatactttgaaaacattagaaatatttgtttttatgatgtaagcatttattttctttgtgtcttccttcctatcagcctgttcagtccttggagttgcacagctggattctgtgatcattgcttcacctcctattgaagatggagttaatctttccttggagcatttacagccttactgggaggaattagaaaacttagttcagagcaaaaagattgttgccataggtacctctgatctagacaaaacacagttggaacagctgtatcagtgggcacaggtgagggatggcaggatcatgagcactccaggaagaacttgcccttttcagtctttgctgcacactgttgagcatcatgcacatagtggccacccctgcggggtgacttttccacatgtgggagttttgttttgtttctgcaggagttttgaggcaggttttatctaaatcatttgaggtttttgtttgtttttatttaaatatatctctgattgaactgaatgttaatcctggttaaaatgagtgacaaaaatactagaagatgtgtgttaaaacatttataccaaatgaattaaggccatttccttcagttctacttatgtgctgaaaacaataatacctaaagaaaaaccttacggccgggtgtggtggctcacatctgtaatcccagcactttgggaggccgaggcgagcagatcatgaggtcaggagttcgagaccagcctgaccaacatggtgaaaccctgtctctactaaaaataaaaaaattagccaggcatggtggcgtgcacctgtaatcccagctactcaggagggtgaggcaggagaatcacttgaacctcagaggcggaggttgcagtgagctgagattgcgccactgtactccagcctgggtgacagagcaagactctgtctcaaaaaaaaaaaaaaaaagaaagaaaaaccttctttttctttaggtactattagaccatggctctaatttcctaagtaagggaatattttatatataacagatattttgctgttattcaaagatgtgtaagactgtcagttcatacaacttgatatggagtgagtgtcataaattaattattcccactctctggtctaataacctacaaaattattaggaaagtcacttactagcgttttacctttactctcagtatttctttattccttttacttcttcttcagccttctccttttcagagctttcagagtttcttatggacaggaagacctagagaaagtatttccccttctccttgatgatagccagaatgaggacaagaaagtattggagagacttagatgattcagaatgtaaattaaccagtgtagctactggaagctgtcaattcttctctttcaaaatgtgtctgattcatagccatattttatactttaacattaaaggtatttacttgtatttgatcctttacctttaagaactatttgctctgtagctgaaaccatcacacttaacaaactaacaaaaatcatgtttgtgttctaggcagatgtattagattgttaaatggttctagcaggagactttttaatagtgtagtaccaggctgggcatggtggctcatgcctgtaagcccagcactttgggaggccgaggtgggtagatcacctgaggtcaggagttcaagaccagcctgaccaacatggtgaaaccgcgtctctactaaaaatacaaaaaattagctgggcatggtggcaggcacctgtaatcccagctacttgggaggctgaggcaggagaattgcttgcacctggtatgtggaggttgcagtgagccaagatcacgccactgtactccagcctgggcgacagagcaagactccatctcaaaaaaaaaaaaaaaaaaaagtgtagtactgttggctttttcgttcccctagtgttacacttcctttccctattacatagggaaaaggtaatagggaattcctattccctattactactaacctgggttagaaaagcatatattaataaagatattaatgtaaaagtattttgtaaactttaatcactgtggattgtgatttttcatatgttctaaaattaagttattttattgcttattgctttctacttttggtattatatttaatttagtagtgatatcatgattttattttcacttctaggtaaaaccaaatagtaaccaagttaatcttgcctcctgctgtgtgatgccaccagatttgactgcatttgctaaacaatttgacatacagctgttgactcacaatgatccaaaaggtaaaactgatattttcattatagagattgatcataagcttttgtcttacaaaaaggtatttgttgatacataattttaaatgtggacagtgataaaaatacagtgttatctgaactattcttaatggttagttcaaaacctatatgccctcattttattttcagaaaactttatctctgtaccaatggggaaaaaaaagtgaagggatcatttcctgaacatctggtcttttgaggttcacaaatattaaactacacagaaaagatcttttgagacattctaaatagtaatatatataatcctatctagtatagcaaagactggaccaaaaggccacttgatttttttagttttgagatggagtcttgttctgtcgcccaggctggagtgcagtagcatgatctcaacctgactgcagtctccacctcccggactcaagctctcctcctgcctcagccttccgagtagctgggactacaggcgcataccaccacacttagctaatttttctattttttgcagagacagggtttcaccatgtcacccaggctgggcttgaatttctggactcaagcaattcccccacctcggcctcccaaggtgctgggattttaggcatgagccactgtgcctggacaaaaggctactttattcaaggagataaatatctggaaaaagtggcacatgtaatttaattcaaaacttaataagcatcttaggataggtaagtaggtattttattaagacgtttagtgttaatcagaaatatttgattaatgagaatggcttgtctgccttttatagaataattttttacaacattttgtaagataactagaaagacttggaagataatattgaacataacttgcaaagataaatgtttcagagagcctgtatattattaagtaatgtatattactacatatttttggtataaatgagaaatgtttggttataagtgagaaatgtcttgtgaaagttttaattctcgatgacatttttatagaagttctgatttaataaatattagaggaaattatatttcatagataattcccgccgaaaaaatattcagcttttatttccatacctaaaaagatgacacaatatcagtgggttgattcagtttgctcattttaagaatgctatggataatctattaagactttatactatttattagcagtctacttcattctcatttcttcactgtttcatcagaggagggggagatggaggagaaagagttttaatagttatttttccttgaatgccaagcatcatgccaaatgcctcagagagattatctataatcctcacaacaacggaaactatacattttttttgtcaggaaatagagcctcagagtgaaaggaactagccaaggtcacccagctggctttgggcaaagtttgaacttggcctaagtcttggattattttggagccttttccactatacaatctctttgatttgaaacattagtattacagtattacagtctgaaaacttcatccattttcatttaaatcataccagaataaaatacagtgacttttgccttcccagtagagctttcacaatcaagtaaattgattttttaagccattttagtgaaaatctaacaaattgttatgctgatttattctgaactataagtcatttaaattttaactttccatatgttggcagatgtcatgtgtctgtttactttttggaggctcttctaacagtaattgttaccaacagctgatgatccagcctgtaccctcttctagcttcacttgaaaaaaactcaatccttttactagtaggaaaggaagtaggaaaagattaaggttcctagtgttttattttcacatatctaaatgttgacatttagaatgtgtgctaatacattattgttatggcaaaatatattgcatatgtatatgaggagagagatttaatccttttttatattttttgtattacataatttgatacttagttgtcttttttaattctaagacttttggtgaaaatgtataggcttatcctgcatttatcattaatgaaccttcagacactttaatctataatgaagcaactctcgatttaatttgtcatgtttactgactaaagagttcaaccataattaaattttaaaagcagttaaatgaatagcatagtctctgattttgtgtcatattttttatttttttaagcaaccaaactgaaaaaactgtaaaacatttaactctgtaaaatttaactctgtaaaactgtaaaacatttaactctgcttgtcatctaaaatagaattttgtttataaattccctgaaggtctacccctgttctaaaaattacccttactccccataatgggtttattttccattaccctttgttctagaagtgtaataacaatttaactagagagtaaaattgtaaagtagatgatcctgaatttagaactttccatttttatttgttccccgccccccatttcgattaaatccaatctgtattatgtctctttccctcagggtcttctctgttagaaaatctgataaacaaatccatattaatgttacttttccaaggagacatttgtgttctatatatgaaaactaccatttactagtggtgtgaccttggccaacttaccgaacctctctgccttggtttccccccataaagagataatagaatctatctcatagggttagcatgtggattaactgagttaatacatgtaaagtactcagaaaagtacctcgcacatgaaaatagctaacattgattgagcgtttactgttaatgctattcaggtatcacagcagtttgggagtaggtgggtcctttagcactccgaaatcaaactgtgaagatgctcacttaatccctatctgtaggttgcttctgaccccagtggaatctttccatattgattgatgcatttaatagacgtttgagcaccatttgccagccattgttctgggtactagagataaaggatcaagtcagacaaaaaccaaccctcatggaatttatattctaatgggaggagacagacaataaaataaatgttacatatgataagtggtagtatatgctatggagataacttggctgaaggagaaagagtatccaagttaattgttgttttatatggtacagtcagagaggcctctctactaagggaatgttaatcagagacctgcaggaagggagtgagccatgtgaatatctggaaaaagtgtgttcaaggaaaaggcagaagctaattcagaggctccaaggtgtgaatgtgcttgaggttttcaggatacagcaaggaggtgaggccagtgtggccagaggagtgaggcagtgaggagaggagtggtgggatatggcatcagagcagtcatagagagcttggatataaacttggaatttgctgggcatggtggctcacacctgtaatcccagcactttgggaggccaaggcaggggatcacttgagtccaggagtttgagaccagcctaagcaacatagtaagacctcatatctacaaaaataataataataataattaaattagctggccttggtgacgcacacctctagtcctagctactcaggagaatgaggtggaaggatgcattgagcccagaagtttgaggctgcagtgagctgtgatcatgccactgcactccagcctgggcaacagagtaagacattgcctcaaaaaaaaaaaaaaatagaaaagaaaaaaagaattggcactgcggcactctcaaattatcaactgctggtagaagtgtaaagtatagcctttctgtaaagcagttggcaatatagcgtgtatctagctttaagaatgtacttgtactttggcccttcattccacagccaggcatgcattttaaaatgttcatcatctgcatcattgtgacggtttgtttcttccatatttggagataaatttatgtgtagactttttttttgtaagacatagttgataatgaaaatttatttaaatggtcttgcaatgatttaagtattcaaatgcttaaagaaagcattgctggtacaaatatttctatttttagaaagggtttttatggatcaatgccccaagtgtcatcagagccattggtgttttcatttttaaaatgtcacctgtaaaatgggcattatttatgtgtatatggctttttttggcattcctgataaatgtattatataaagtctatacattggataataacactagtatatttaaacttacagacttatttgtaatgcaaaccaccattttaatgtactgtaattaatatggttataatatatatatagtccttctgtcctaccatcacacaactttttgtgtgtgtaataaaccgcttttggtttgaaaatatttttcgaggccgggcacggtggctcacacctgtaatcctagcactttaggaggccaaggtgggtggatcacctgaggtcaggagttcgagaccagcctgaccaacatggtgaaaccccatctctactaaaatataaaattagccaggcatggtggtgcatgcctgtaatcccagttacttgggaggctgaggcagaagaattgcttgaacccgggaggtggaggttgcagtgggccaagatcacaccattgcactccagcctgggcaacagagtgagatgccatctcaaaaaaaaatatatatatatatattttttaaaaaaagaatgtttatcctggcattattcttattagcaaaatattaggaaaaacataaatgttttgaccaataataaggaaatatatgaattatgatatctatgtaatatagtaatggggaaattattaaaaatgatcttttcacagactattacatgggaaaatattcacaatataagtggaaaaagataagcaaataaggctttaactacagtgtgatttctaattttgtaagatatggatatacacacacacatacagatataagatctagagatatatagagattcaagatatggatgaagatatatatatatatatgaagatatatactcatatacaccaaaaaagttgtaaggaaatgcatgacaatgttatatcatctccaattgataggattatcagttactttttttttctttagactttcctgtattttccctgactatacattagaatcacctggagatctttaaaaaactgcagatgtctaggctccactcaccaaaagttaagaattaaataggctggggtacaggtgtagatactttttaaataactgcctgggaatttctaatattctggttcagattcatagcacacacatgaagaataagcatattatatattaaatgttgaggtgtttcttttgttatgttaaatgcttacctacatactaaagtgcttttttgttttccaatttaacacgctggagttttgtttgttgttgtttcttttagaactgctttctgaagcaagtttccaagaagctcttcaggaaagcattcctgacattcaagcgcacgagtgggtgccgctgtggctactgcggtattcggtcattgtgaaaagtagaggaattatcaaatcaaaaggctacattttacaagctaaaagaaggggttcttaactgacttaggagcataacttacctgtaatttccttcaatatgagagaaaattgagatgtgtaaaaatctagttactgcctgtaaatggtgtcattgaggcagatattctttcgtcatatttgacagtatgttgtctgtcaagttttaaatacttatcttgcctccatatcaatccattctcatgaacctctgtattgctttccttaaactattgttttctaattgaaattgtctataaagaaaatacttgcaatatatttttcctttatttttatgactaatataaatcaagaaaatttgttgttagatatattttggcctaggtatcagggtaatgtatatacatattttttatttccaaaaaaaattcattaattgcttcttaactcttattataaccaagcaatttaattacaattgttaaaactgaaatactggaagaagatatttttcctgtcattgatgagatatatcagagtaactggagtagctgggatttactagtagtgtaaataaaattcactcttcaatacatgaatggaaacttaaatttttttttatgtgtccttgcttatagtttagctgtaataatttaaccttgtattcttgtgccatattctgtctttttattacttataaagacaaaccaaagtaaatctgaaaggagactagaagctttgaaattattgtttgggggttttataaaagcaactactgtcacctccatccagattcttttaaattattgatccatccatagtatatattgctactcattcaagaatcctcaataagtattgagtatttaccatatgttgggatactgtgggctctggagagaggagggggcaatagagctaggaattaagaatcagttgagtaaaatgtgtaatatttattccccattaataactgactaggaaggactaaaagccagaaaggggatgaaaaaaaaatccttaattcagggccgacattatctacttaaacaactttgagatatggtcttaattattttaaagcagaataatataattgaaagtttatagctaaaagagactatataggtcatttagtataattcttcattagtttacgaaccacaaaattgcaaataaataagctatgaactttgatgtacactataaatctccttaattctataaatttgtgtctgtaacctgaatagtttgaaaacttctttaaaaatctcttgtatttcatccgggcgcagtggctcacacctgtaatcccagcactttgggaggccgaggtgggcagatcacgaggtcaggagtttgagaccagcctgaccaacatggtaaaaccccatctctactaaaatacaaaaattggctgggcgtggtggcactcgcctgtaatctcagctacttgggaggctgaggcaggagaatcgcttgaacccgggaggcggaggttacagtgagccgagatcacatcactgcactccagcctgggcgacagagcgagactccatctcaaaaaaaaaaaaaaactcttgtatctcaatatttttaaaccacaggcctaaataaaactaattttgctcaagttttctcaacctagggaaaaagaactatggttccatattcaaaataaatattatagacccttttcctaagtaggattttgtggtttactgattgggtaatttgatcattaaaattatgtgaaatctgcccgggcacacctcatgcctgtaatcccagca

SEQ ID NO: 19 - DNA, 29764 bp, Homo sapiens
cacagcagcctccgtctccggggctcaagaattctcacgccgcagcctcccgagtagctgggattacaggcacgggccaacacacccggctaattcttgtatttctagtagagacggggtttcgccatgttgcccagcctggtctccaactcctgagctcaagtaatccacctgcctcggcctctcaaagtgctgggattacaggtctgagccactgcacccagccagcctttgctgcttttgttcctgcaatttggaacactgtccccatcccagcctctcacctctacccctacctccttcactacctataccttcctatccatccttcaagaccccaaaaaccatccctgattccttcagaaaggcagtttattgcctatcttatcagactgaaagcagtggctgtgtcttatttatggttaattccctagaagctggactgatacattccatttaactaaaattcgtatcaggtgcttcggactgcagacaagcctatcacaacccagaaggaagaaacagggaaggcacctgggggctgccaagcaatgaggtggggggtaggaatcatgaatccgcatatttttaaaaactgccccagatcctgatgtaaacggtacaagagagtctgagaaacacagggctcccctcaaacagtcctgacttcagcattcctggaaaaatgaaaatcctttccttttgcctctaatgctttccctgctggtatcccaggttaaaaaaaaatagataaaatcagggggatttttctgggacttggctgggctgggaaacaagcctgggttctaatacaggctcagcccctgacgtactatgggcccctgcccctccttggggcctccattaccacggccacccccacccttatcaattgtgtgcccctgaggtagtgactgtcccgctctgagcattagtttccccatcttccactagtcgtcgtcagctctgacgctctatgagctatgcatacccgtagctccccgccgaccccgatggtcccctcccctccttcccaaggtccatccgccagggtgcagccgacgcactcctaatgctaaggccgccctctcatcgaccgccccttcctggcctcgactcagcgccaaaggtatgggtctctgccccgcctgctctttaagcctagccggggcggtcagcgcaagcgcactgggtcgcatcgaggccccgccccctgagcctgggtagcggcgcgagggccgggagaaccgttcgcggaggaaaggcgaactagtaggttggggcggccacggcggccggcatgggtcacgtttcctcgggaggaacgatgtgagggaggggtctggcaagagattggaattccggaggccgggagaccttgtggctgaaacccttcgtaggagcggggcaactagtgtctagtgagggggttgggctggcgcgcactgatcccagactttccggatcttctgcctttagatcgggccggtgtcggggcatgtaggccagtgagactggagccagttagagctacaacggggagcgattagggccaaactttgtccagggtggaagcgagcgggcccgtgaagtggggccagcctgggcagccgaccgtgtcgttgcctcggggcctttccaggcactggcctaagtcctggcgataaagtgcgaccgatttccttgtgggcgttttgaggctttcggtgatctgacccgtctgtcattcattcttcattcattcatgtgatgaatgaatacagtactaagcgcggctaattactaggtagagaagtgatcaagacaaacactgttcctacggtacagggaaaagtgatgggctgtagaatgtagaagcccggggcggagaacagggacagcttccggaacgaaatcgcgagcccagatcaggagtggtggcgagagttccaaagagaagacagcacgtgccaagtcctggaagggggacagaggccaacatatcctggtcactgaagacacctgactctgaatctgtttcacgcccagggaagagatgacagtggccggggctaggctacaaactctggaaatggagataaataaaggaattcaaagtactatatacttaggcagcaaaatccataggatttggggagagtgagatgtaggaaacaagtactcaaggcttgggtacctgggtggggttcatcagagaagaagcagatttgtgggagacaacaacaaattctattctggttgtatggagactcgcaggaaaaaattggatattctagtttgaaggtaggaaagtattgctgtgaagatgtagatttgaatgtcatcagcaaaacataaataaagccaagggagggttgaggctgtagaatgagaaaaacaaagggcccacttagcaccttcatctgatttcttttctttctttctttttttctttttttttttttttgacagagctttgctcttgttgcccaggctggagtgcaatggcacgatctcggctcactacaacctccacctcctgggttcaagctattctcctgcctcagcctcccaagtagctcggattacaggcatgcgccaccaggcccggctaattttgtatttttagtagagatggggtttctccatgttggtgaggctggtctcgaactcccgacctcgggtgatccgcctgcctcggcctcacaaagtgctgggattacacgagtgagccaccacacctggcccatggtgattatctttatgtcttatcctcctccatatccccagtacctagtcaagggagtggcattaaatgcaaatcagtgtttgccaactaaataaaagcccaacagcaaacagatgttggaatttcagagttgtggaacgatgggggctcatggagggtttcattactctaatgtcaaggtaatgggttcttgtcctggctctgccactaggcttctgtgtgacctctgacaagtctcctcctacctataaagagagtacagccaaaaaatggtctcatgtatagagcttcaaacactgctgataaatttcacactgatttttctcttttaatccacacagcaatcttacttgaaagggaagtcggctgggcggggtggctcacgcctgtaatcccagcactttgggaggccgagagtgggggatcacgagatcgagaccatcctggctaacacggtgaaaccccgtctctactaaaaatacaaaaaattagccgggcatggtggcaggtgcctgtagtcccagctactcgggaggctgaggcaggagaatggcatgaacccaggaggcggagcttgcagtgagccgagatcgtgcggctgcactccagcctgggcgacagagccagactccgtcaaaaaaaaaaaaaaagaaagaaagagggagggagagagagagaaagaaagaaaaagaaggaaggaaggaaggaaagaaggagggaagggaaagggaaagggaaaaggaaggaaggaaagaagggaaagggagaagtcgtattattatggactcaaacccaggtctgtctgtctgtctgacttgaaccttgttctttactatgattgccctcatgtatgtctcactcaacagggatattatcaggaccctcttgagatcacatgcgcattctttcaaagcattgtgctgaggctggcagactttcataattggcctggcactggctctgtcatggggacagggggacagagctgaatgtgatggaggtttcctattattctctaactcccttcctggggaccactgagttgggcaaccatgttctgttaaatggcaacagggcagaacaaaattagtgactgtgtttccagatttttacccagatcttaaactcctgaggcctgctgaaaaatgaatgagtatcagggtgtgagtttgtacacctctgtatgtctctgggcaaccaatcagacaacttctcctattacattggacacttgggtttcagcaatttccatcttgctaatgtgatttctcaaaaatattttctgtcttttggtgctttgatgataaatgtccatatatggaatgtagtcatttcctgctactaagattccttctggtttgtataagggaggagttcaccttattcgcatttcatggtattccacaaagagctccctcccccttcccatgtaatttatttgagatctgctgacatgagttgttggagcttgaagggaattaataatgtactgcagtgactcctatcccaggaaaacttgttaaaaatacaaagcctcggctgggtgtgatggctcacgcctgtaatcccagtactttgggaggtcgaggcgtgtggatcacaaggtcagaagatcaagatcatcctggctaacacggtgaaaccccgtctctactaaaatacaaaaaattagccaggcgtggtggtgtgtgcctgtaatcccagctactcagggaggctgaggcgggagaattacttgaacccaggaggcggaggttgcagtgagccaagatcgagccactgaactccagcctaggcgactgagtgagactccatatcaaaaaaaaaaaaatacaaagcctcaacccctccttcccatcaggcctcttgcatcagagtctctgggatggggcccaggaatctgtattctttcccagctccccagaatgttcagccaggtttggaaactgatctatccgattcttcttgtttcacagttagggaatctgtagctctgggaagggaaggaacttgccccagtcacatctgatattagtgcttctttctccaatgaagagcctttaggctgggagtccagagacatgggttcaagtccaggctataccagtcatcacctcgggcaagtcatttcacctctccaagcctctgcttccttactgtgagaataatgccattgtgttgggaatcaaaagagagagtggcaatggaaatgctttgtcaagctttctattttgtgcacatggaagttgttaagagctagaaccagccagtgttcactcctgtataccacgctgttcccttccaacagaggtcagggtcctgctgtgttgggggtggccgccagccagtttcggtggttgctgggcttcaggccatctgttaccaactctcttctctccatcttttgcaggtgttgggatggccaccaactgggggagcctcttgcaggataaacagcagctagaggagctggcacggcaggccgtggaccgggccctggctgagggagtattgctgaggacctcacaggagcccacttcctcggaggtaagcccctagctcctccccacagcattcaccatggcccactgtctggccccggccaggctgagggtcactcctttgcatcagggaccatatctcttttgccttattttttccagtaactaaaattgatctctagaagtagaaaataaaaaggcagtgtcctggagtaatcaaatttaaatatggggtttgaagtgtgacttaggcaaattacttaaccgctctcagctgcagtttcttcaggtgtaaaaatgggataataataggacctacctcactggtttggtaagagaattacaggatgattcatgtgaagcactttgcccagtgagctattactgaaaaccccataatcactctaccttctctgtaactggtttgtgatatatgctttcaagcctttctctgtgcatttatatacatagataggtatatatagaaatacgtcttttttaaaaaacaaattgtatcatatatattattctatgatatgtttttggttttttatttgtctgtcttagaactttctaatgccttctattagggtcatcttcattctgaggcatagtattccagatgtgggtgtatcacagtttagcttccctctactcatgtctatttaggtgatttctcattattttatgaccataaatagcattatagggaacatccatgcatatgtctgttggggcacctatgtgagtgtttctccaggttcaatacgtaaaagtagaactgctgagtccaaaccacacctttttaaaacctcatccttagggaagaaaaaaaaataaaaaaataaaatcttttttaatttggtgactaggtaagacattttcatggttcaaattcaaatggtacaagagtttaccctaatgcagcttgtgttttggtttcttgttgatcctttcagatggcttattcataacaagtaactaataacaagtaaatatattccttggggcctcgctctgttgcccaggctggagtgtagtggcacaatcttggctcactgcaacctccgcctcctgggttcaagcaattctcccacctcagcctcccaagtagctgggactacaggcatgtgccactacgcctggctaattttttttttttttttggtagggacagggtttcaccatgttgtccaggctggtctcaaactcctgacttcaagtgatccactggcctctgccccccaaaattgctgggattacaggtgtgaaccaccgtgcccagcctccttttgcccacttttttttctttaacttaacagtacaccttaagaccatattggtgactaaagagctgccaacatctcttctttttttagccagacccattcttttttgtctctgtgtgcccagaacctacacaggcctgatggagtccacactcagtaattgtttgctaaggcccaagtaaatgacaatgtctgtcacctaaggcaggctgatggtatggaatagaattgcttgggctgtgaacctagattttgtgaattacttgtatgaatctaaaatgaagcattttctcttccacgcttttgtttcttctgttaatcaataggtaccatgtgaagatccaacacttggtctttctgggaggttatggagcctagaaaaggcgtaatccacaatgagatttctaatccagaaaaaaagtcagaaagtgtacgtgtgtgtgtatgtgtgtgtgtgtgtgtgtgtgagagagagagagagagaatgattttactccaaatctttaccaaatgcctgctctatgccgggccattgtaggcactgaggacagagaggtgagtcagccagagccctagcctctggggctcctggtctagttagagaacataccacaaaacaaaattaaataacatcagctctatgaggaaacacaggcagtctgctacacaaagttaactatcatttattgagtatttagtatgtgccaggcattattctaaacactttggaatcactgactcctcaccacaacagagataaggaaaccgaagcccagagaggctaagtaacctacctgaggttatttaaatggtaaaataattggcagagccaggatgtgaacccaaggaatttggctcctgagtccatgctctgaacatccatgctttttttcctctcccaagatactatataggacagctggaagaaagagtagttcattatgattagatgattgagatggggggtggtatttgaactagaccttaaaagacaaatagggaaaaggaacagcatagcaaggacccaaaagtaggaaaaggcaaaaaaaaaaaaaaatgttcaagagaatgcagctgaaatgcagggcgcataagtggatatagtgggaaagaaggcaggccagcgtcagacagccgcggggccttagctactgggtggaggaggagtatgaactttatcctgtagatcagagctgcaaactagccatatctaggctgagtatttggctcctgcagtgtttggggagttaatttgttttttacaataagtaatacgttcaaatacatttgaacagggcgcaaaattcaaaagggtattcagtacagagtaagtccccttcctccagccactgtttccctgaccaggggcaagcattgttaacagttgttttcaccagagtatttttaaaaagtcagagccaacatttaaaaaaaaaaatcatgaaattaaaacaaaaaatctagcaacccaacttgtcttgaaaaattatatcatctggcaactctggcctactttcctgcatggcaacaattggctagagcagagttctggctgccccctttagagaagatgcaagtacttctttttgccacaattcctatcactccctgttgcttcctggctacaaagcagaattttgtcatgcacgtgctataggtttttttaaatagtagaaaaatgtttctttttttttttttgagacagagtctcactctgtcgccaggctggagtgcagtggcacgatctcagctcactgcaacctctgcctcctgggttcaagcgattctcctgcctcagcctccagagcagctgggactacgggtgtgtgccaccacgcccagctaatttttgtatttttagtagagatggggtttcaccatgttggccaggatggtctcgatctcttgatcttgtgatctgcccgcctcggcctcccaaagtgctgggattacaggcatgagccaccgtgcccggccagtaatgtcattttatgttaatgtctctatcaaaagtgagaagactgtgtgtttcagcctttagcctgtagatagcagagaacagctataaactattgatcctaaattcaggagggcataatgagccctgggacagaggcagagggatgtcttagcagaaaaactctgagtttttgaggccaagatgagacttgttgggggcagcagagctctatgtgttcaagccaaggaaatgctcctgtagtcatcacatagctactcagggtattaggtcaccccttatgtaatctgcagtcattcccattctaactcataaaggcttcagactgaataaaccttattctcacaaatagccttcctcagttttatcttagatgctgaggccagggcagtggtacacacctgcagtctcagctactggggaggctgagacaggaggattgcttgagcccaggaatttgaggctatagtgcactatgatggcacctatgaatagccactgtattccagtctgggcaacatagtaagaccccactccaaaaaaaaaaaaaaaagatgctgagaagggtaatttagaaattatctaccaaaatttaaaatagatttacctgaattacttgatatttttacttttagaaatttagaatgtatggactttctcatatatgaaaatatctagaatgtatattctaggatacccattgaaacattaatggtaataggaaaataaagaaaccacctatatactagattcaataaattatggtatatcctaagagtagaataatatacagcaattaaaaataatgaaaatgctctatttgaacatataaggaaatatttaaaaagcacaatgtagtatagtttgctatgggggtgaaaaaaagagaaaatatatgtgtatacatatatatcaatacaaagatgactggaaggatgtgtgagacactagtctgtcatttgcctctggggaggagaactgggtggctggggcccagtcttttccccaggagactggaatgagagtgagacatacttctcactatatattcttatgtctcttttgaattttgtatcatgtatttgtattacctgttaaaaaaataataatattttggctgggcatggtggctcatgcctgtaatcccaacactttgggaggctgaggcaggcagatcacttgaggccaggtgtttgagaccagcctggacaacacagtgaaaccccatctctactaaaaatacaaaaattacctgggtgtggtggcacacacctgtagtctcagctacttgggaggctgaggcaggagaattgcttgaacccaggagatggaggttgcagtgagccgagattgtaccactgcactccagcctgggcaacagagtgagactctacctcaaaaaaaaaaatagtactttaaaaataaatatctaaataccaagttctaacaccgtaaacttataccaccataatgacaaactgatattaactcaaaggttaaactcaggaatgctttataatacaagtcacaagattttcttttcatctcttacccaagttctagttcagttgttggcagggatctcagaatgcacttttcccttctagaatcagtgtccttgatggtatttgggtttctcgtctatgataaagtccaaagaatgcggaatgcagctgaactctaggcctgttaacctgagtcaccatcactaacattggtggaaaaaacactcctggcttctactaagggaaccagagttcacttgtcctacccagtaaccaaatcaaaatcaaaaggcaaggaaactggagtgtgagctcctgatgcatggaagggcctgggcttgaacatcaaccagcaaggagcaggcttccatgtatgtatgtgtgtgttgtgtttagtaaccatcctggttcaaatcccagcaccccgttactagctgaatataattttggatctgttaactgttctggttcaaatcccagcaccagttcctagctgaatataattttggatctgttaacctctctttctcaggtcccgtctctgttaagtgtggataataatagtatcttcctcacagggctgaatgatgaatctatgtaaagtatttaaaatagtaccttgcacatagtaagtgctcaataacttgtgggtttctttttgttatttgcattttgcttttttgcttctctctcttcaatacgtagagataaactatcacagaatctggaagctctctgggttccactctcccccttccactctcccaaggtaaccactaatctacagttggtgtgtcctcagtaaatataggccagactttccatgggattccatttgcaggaagacaacccgttcacaggtgccctacccctgtcccattctctcttcttgatcacaggtggtgagctatgccccattcacgctcttcccctcactggtccccagtgccctgctggagcaagcctatgctgtgcagatggacttcaacctgctagtggatgctgtcagccagaacgctgccttcctggagcaaactctttccaggtaggggacagtgaagcattggggggccaggagctgccagagccaaggaactggaagattgcagagccgtgaggtgttactgtgtcagctgacttggtgggatagaggaaaggtacctccaaagaacaaaaagtcataggagtcaggaaagctggcttctaatcctggctcgaccagttatttatatggcctcaagccactccctttccttctctgggcctaaggtttcttcatctgaaaaatgaagagactggcttaaatccaagatccctttattgttgacattctgtaatccgtgacaccctactttgaagactgatatttccatttggaattaggggaagtcagcctggttttggaggaaaacagaggtagggaaggttattgggttaaagtcagattttctacttctcctaagcagcgacactttcttgtcacctcaggcctctcatctttggatgggatggggtacagactgggccacactcagggcatgaggaagcaacctctgaaatggttcagcccatccgcccttctctgtctctttcccttgatctttttttttttttcttcagattctggggcaatttcttaaaatttctttatttattttagaattaaatatatataggctgggcgcggtggctcaggcctgtaatcccagcactttgggaggccgaggtgggtggatcacttaaggtcaggagtttgagaccagcctggccaacatggtgaaaccccgtctctactaaaaatataaaaaaattagctgggtgtggtggcgggtgcctataatcccagctacttgggaggctgaggcaggagaatcgcttgaacccaggaggcagaggctgcagtaagccaagatcacgacactgtactccagcctgggcgacagagcgagactccatctcaaaaaaaaagaaataataatacatatatatatgtatatatattcattgtagaattaaatatctagaaatattatgtatttacatatacatgctagatgtgtatatactgtacaggttgagcatccctaatccaaaaatccaaaagctgaaatgctccaaaattcaaaacttgttgaacaccacatgactctaccagtggaaaacgccacgcctgatgtcatgtgacaggtgcagtcaaaacacagtcaaaagtttgtttcatgcacaaaattaattaaaatattgtataaaataatcttcaggctatgagtataaggtgtatatgaaacaaatgaattttgtgtttagacttgagtcccatccccaagatgtctcattacgtatatgcaaatcttccaaaatccgaaagacttctggtcccaagcatttcggataagggatatccaacctgtaattgcatgtctttgattaatttttcaacagaaattagacttttgttggagacaaaatcttttaaaaatgtgtgggtgaatatgagaaggggtcataatggtaagaagcttggaaaccattgacttgtagccaaaaacccaatgagtcatgaatgtatgaatctgcccaccaccttgcccctgagctgtttcttggaatgggcccagctttgtacctgcaatcctggattgtgggaaacatgagcagcctggcttataaccctaatgatgcaattatgaaggagacttgcagctcatctttgcaacccctgccttctctgttcctctctcctctcatacacatataaaccctagttcctaagggagaagagccccctacaaaacatgaaggggagcacctcttcagaaaaaggaaagtgtgtctcaacttccttggaggctgaagcccagctgggactctcctcctaaccaagggctggcatgagagagctcacccttgggagagagctggctgaggagcagaggaacttcagggcaggcctgggctacttggcttccccccactggtctgctgtgacgtttctgtaacaaggtgattcaggcttgagcaggtgtgcagaatccaccctgaatctcaaagggcagtaagtgtgatgttaatcacctgtggattccttcttactgtggctcttgttgagacttcagaaaaccatactggataggcccctaacacatgtcacatgtcatggcagtacactgagctgtgacgatgagcctgcatagacacagccattaccttcatgaggtttatagtcaaacaggagagatgacactaatcatcacacaaagaaaatgtaaaattgcaactgcagtcagtgctgtaaaggagtggttcttagttctatgagaacacatagtagggggatctgactcagatagggaaggcttccctgaggaaatcacatcatatgaggactagttgaagaagaaacaaacaaacaaaaaaaacccaggatatttagcttgggtcagaaaaatcttattggtgcacagggcataactgctgtcttctaatctccaagggctgctgtggaggaggaggagaaggctcaccctgggaggtcgcagagggtaggaaaagctatggagagttagtattaggtcaaccagaaggcttgccaaccatcagagctattcaagtagaatagatacatcatgtcattttctagctcttcttcggtgcagtatgttcaattctttgaatgtaatatcttattggctttttaccacaactctatgatataaatataattatattctccattttaaagatcagcaaactgagacacagagaggtgaaatgattccctgaggttgcctactagtgagtggtagagctaggatttgaacccaggtctagagttggattcttagccactgttctctaccacattggggcggacattcaagttttggccagtagactcagagaggattcaggagtcaatgactgaggatgggactccttgaaattttaggtccaattaagcctgcaaaatgttctctgtttcttcctccagcaccatcaaacaggatgactttaccgctcgtctctttgacatccacaagcaagtcctaaaagagggcattgcccaggtaaccattcccagccctactccagtctgtaacctgtccctcccatctctgtttgttttctgttttgcttgaagaatttggtccaggccctcagctcatgggaatctgcctctcactggtcctcactgggtttatcccagtgaccaattctaggatgaccagaagaatgattccactgggcttgggagtgtttgctggtacctctaatctctgtgtagagttcatggtacctgtgtgctctgtggctaggtcctcagagtcagtccctgggcaggtactgtcagccttcagttttccccacagactgtgttcctgggcctgaatcgctcagactacatgttccagcgcagcgcagatggctccccagccctgaaacagatcgaaatcaacaccatctctgccagctttgggggcctggcctcccggaccccagctgtgcaccggtgggtcccctgggcagcccccggcatacctgtggggtgacatgctgatgggtgtacagtcactggctaggccagggaactccagctatgattgtgctttcctgggccccgggtcacatgttgcccctggccaccccgacagcagtttccacttgtaatgagatccttggtatgtcagggagaaaaaggacctcatagctcatctagtcctgtccctccattgtacaggcagagggaacaatatcttgagagccccagagagaggaatgcagggacttctgtctgggggctgggcctggtagcatccatttctagccagcagtgatgctccaggttgcaatgattttagatggtctgcagcaggattccagacagcacctggaggcccagagtaaggggctccagctcactgggacactagggtaggttggggtggggacagaggctctcaggtctcctccaggcatatacaccaggggccaaggttagggcagcccagcatattccaacctgaagtggatcttacaggaatgtgatgggaggatgctttttagtgctcagctgattctcagagtcatgttgctgtatatatgaggtcatgggcagaggggtcttccaggtccatccaattactgaacagccatctctcttccaacagacatgttctcagtgtcctgagtaagaccaaagaagctggcaagatcctctctaataatcccagcaagggactggccctgggaattgccaaagcctgggagctctacggctcacccaagtaagggtgtgaaaaggtagcaggaggatcctgctttagtttcagcattcatgggtttagcaacttcttttcttgccagccatcattagagaataaggggatttttctaggaatagaaacttatacctttacatgccaaaattattttaaggtttccttcttaaataacagatgctgactatgatttaactttttcttattgagtggaggtcatcattatgactgtcaacaattgcagcttgctgtaatacagtagtgctacctagggttagagaggcacgcaaggctgtttgcctgcgctaatagctctgactgctaggctttaagttcttagtcatttccttttttttttttttttgagacagagtctcactctctcacccaggctggagtgcagtggtacaatcttggcttacttcaacctccacctcccgggttcaagcaattcttctgcctcagcctcctgagtagctgggatttcaggcgcatgctgccacacctggctaatttttgtatttttagtagagacggggtttcaccatgttggtcatgctggtctcgaactcctgacctcgtgatccgcccaccttggcctcccaaagtgctgggattacaggcatgagccactgcacccggcctctcattcattttcttcatagttttcttgtctgtttcccaattctcagctcttacttttgactgctgttggtatgcttgaatttggaatcctccaccccccatgcccatgcctcccttctgatttgctgtggtttgggaaaacaaatgatccagattgttatgattgggtctgaagagtgtgagggcctcttggatgagtaaatgcataagctttgactacgaaattttatggtatcctttttaactgcttagaggcattttttgctttcttcctatttctcaagtgaagatgttaggtaagtgattttcagatcatcgaggggccgctatactaacagttattgcaatgttaatatagcattaatagtccttaatgtacacttactagtgctacaccttgtgctaagctctgtacatacaggatctcattgaattctcataataagctctctgaggtcaatactgttcaactccctcattttacagatgaggaaactgaggttctgagaaacgaagtgaattgttaaggctaagtgatgagttggtggcagatcccaaagtctacctccctctaaaacctccactcttaatcatgctcttacctccaagggagcctctctgtccttgctaagcctcactaagcccaaagaaacctcagactgtaagcatttagaagtcatcagacaaatattctttcaagtatattggctaggttgtattttaagagagtgaagccaggggatgggtcagctggggaactgctgacagacaaatgctgcagagggttttgcctgccagcctgtcagtaacgtggacagaaaatacttgtgtgtccaaaattaggcactggtaggtaggagttatgtggcacctgagccagaactggcttcccccattgtgagagtgagataggttcttctgctgacatagcacatgaccttggcaagttagttcttctctgagcttcagtttcctcatttgtaaaataggagtaataataatacctaaaggggtgttagtgagaattaaatgagatcatggatctgaaaaatgtttttaaaaatctgtgtggatcattatgtggtactttcaataataataataggccgggtgcagtggctcacacctctaatcccagcactttgggagaccgaggcgggtggatcatcagaggtcaggagttcaaggccagcctggccaacatggtgaaaccttgtctctactaaaaaatacaaaaattagccaggcatggtggcaggcacctgtagtcccagctacttgggatgctgaggcaggagaatcacttgaacccgggaggtggaagttgcagtggccaagatcaccccactgcactccagcctgggcgacagagcgagactccatctcaaaaaaaaaaaaaacaaaaaacaaacccaaataataataataatagctatcatttgacaagtattagttttaattcatacaacagcaaactgaggctaagagagtttgaataacttgcccaaagttacacaaccggtaagtatagaattcatctgcctctaaagcctatgttctctctacttccctattctgcctttaagagatatggttccacagtattgactgaaaaactgcattggtagagcagattaattttcgtcaattatctcatgatttttaaaatttcttaaaaatggaagcctgcaaaatgacttacaatttcaatttagacaaactctcaaagcatagggcctgtggttagaatgagtagaataagaaaaggggactactggtgataaaagtttgggaactgtgatctttttaacaccaatttttttctttttttttgagacagagtttcgtttttgttgcccaggctggagtgcaatggcacgatctcggctcactacatcctccaccttccaggttcaagggattttcctgccttagcctcccaagtaactgggattacaggtgcccaccaccacgcctggctaattttgtatttttagtagaaacggggtttctccatgttggtcaggctggtctcaaactcccgacctcaggcaatccgcccgcctcggcctcttaaagtgctgggactataggcgtgagctaccatgcccagccataacactcttattttatagatgggaaaaccagggcccaaggaacgaaattgccttacccaagtcaattaccaagacacactacaagtcactggcagagcctggactacctacgactcaggggtcctcacccccagcccgcatgcgtccttagctgacaactttcctactaggaaacagactgctgagaactgctcagaactgaaggcaggagaggtcaaatatgttttctgagcccagctctgattgtttagcagttggcaggctgacttaattagctggggcgtgcagttcctctttaacctccagctgccagccttcctcctccgcctctttttggaggtgggccagcctgggccaactgcctccctcccacacacaccctcacccatgagcgggacagtttaggctgcaaagtgaagagcaaagccattggcccttaggactctctcagggcaagatgacttgtgagagcaccacttttagtttgtctctcaggcacccaactcaaagccaagactcagcttaacatcacatctgacctcatgagatttcaggcaaaccaggagaggggacttactaagacctatattttggctaagcagaaaggagtcaggcaaacagagtttagactaagaggttcagccaaggtcaggagaagcagagatagacaagagaggctaagcagaggaggtcagggaatacacacttagaatcctaagccaaagcctagggttccatgggtctcaggaagaagccacagacacaaagcagtacagtcacagcaaaaatggagtttggaggctgagcgcagtggctcacacctgtaatgctaacactttgggaggccgaggcgggcagatcacctgaggtcaggagttcgagcccagcctggccaacatggtgaaaccccatctctactcaaactacaaaaattagccggcgggggtggtgcacacctgaaatcccagctacccgggaggctgaagcaggagaatcgctggaacccagggggtggaggttgcagtgagcctagattgtgccactgcactccagcctgggcaacagggggagactccgtctcaaaaaaataaataaaataaaatgtaaaagaaaaaaaatgcagtttggtactgctgagcattagccctaggaatctcttaggggactggacctatctttgacaacggaaatatgttagctggcagccaaacagatagttccctggcataagcttttccctgagccctcaagcccctgcctctttaagaaatacatgaataatcagagaggaagaagccacataagccctagtgatctcattaatactatgagatcaaatgtggccctgtgtacattataggaatcttgggagggcccaggagataatgtcgttgtttgtagttggccctgtgggtttctgtagggttccatcttgtgtaagaaccacattcctttattgtatcctttacaatctagtaatagagccattagccccggaccccctgcattgttcttttacaaaatgttcctcaatactcccacttgtttattcttccagaaagattttagaattatgttaagttctaagaaaaagtcctcttgggtttttgagatggttttaaatctaaattttaatttgcacagaaattcatcaacccatgacatcattacaatatttcatctgcccactggagaagggtcagaggcatcttcatttttgaagttttctattttcaggaaatcatatgtgatagcatcaggtgtctatgcctgaggtaatctcaaggttcctgagagagggaacatctgttctttcagggaagcggtgttcttattcttattccaggaggtggggcggtatgggggttgaggggagaaacaaaagaagaacaagttctatagtagcctcgggccacctgtgctctttccccagtgctctggtgctactgattgctcaagagaaggaaagaaacatatttgaccagcgtgccatagagaatgagctactggccaggtaagtaaaggaagggggacttctaggtgtggctccaggattaggggtggggcactcagaacatagcatccattccctctggctcttgcccatttttcccaggaacatccatgtgatccgacgaacatttgaagatatctctgaaaaggggtctctggaccaagaccgaaggctgtttgtgtaagcattcccaagaatccagtggaaggctggtttatgaaactcatcctgccaccctcttccccaaaatgattctttcttctgggagatgtgatggcttgcttccttctctcataattcctgaaatatctcatcctcccaggaaattttggagaaagccagccacgctgtgcttctatcagagctgttgacattctggatcagggtctccttagagatcatcttagttttcatatgccctaagttcccaaaagttttcttgcctctcctagtaaggtgaggtcaggcctgagaagctgagctgggcagtcagggaggaagaggagcagctggctcatgctgtgattggtctggatgccactgtctgagctcgagcctggatttgtgttccaagccaagccttatccttttctctaggggccaccaccaggtagatttggtgctacatatttgggtagcattgcagcacatatatttagacctagacctttgtgattgttaaaattaaaactgtccatggaatttcacaataccactcactgtttttcaaaatgtgcttttatcataactaaacaaagtagttaatttacttttcagataaactagacaatatcaaataggtcaaagaaaaggaaaagacatttaaaaagcctgtgtcttaatcagactcatcattttacatgtttgcgttttcaccttcacccctgccattaaaaattttttcattctggtttcagctgctttaagcagtggaaatataaagtgtgttttactacacatggcagtatgattctgctgctcggtaatttcgagccaacatttgtatgcatttaccaaatttgattctagtgaccttcttgttccttctggccttcttagaatgactctaaatctggcatattctaaagtattctgtatggcacacctccctgttttcagtggaagccctggtagtgtggatatctactttcactggttccagtgaacccctgaccaggctcccactgtgggctgaattttgaaaaagccaaattcatcttgatgcaccctgaaatagattgaaccactgaacaaatcagttataatttaacacagcagccttctccatcctgtgttccagggatggccaggaaattgctgtggtttacttccgggatggctacatgcctcgtcagtacagtctacaggttggtattttctgtgagaccattctttgcctcctgggacccacaagagctccacagagacccaattcaggcttataacaacctgggttttccgagtcctcacttcacttctttctcagggagcttgctgctagaacctcctatcctccctcaagccttttgctacctatcactctacacagtcttctagaatttgaatcctcaggaatccacagagcttcagccatttacactgtttccagagatgtgctggcaaatgtttaacaacaatcagctctcactggttgatataagccagttccagcatactgctgaccattttttttcctgccaactcttacctttcctttatctgaatcagaaagttttatcatctcctcattcatgttaatgacagttatatcacctcattttgctatcctaccatgtagtttcattagtttccacatccattatttcatttaaccctcacaaccactcggtgaggcatataattatccccattatacagatggagaaactaacgtttagagagatggagaggcttctctaaggccctacaggaagttcccaggttttctgactttcaggccgatggtattcccattcttctcctctgctcctaacatccacatcatggagaggctaagaagctctgctctcagctgggagatgataaaggaggaaataagtttagaaataccatgggcagtgagctggaggtcatgagcttgactgcctctgtgtgatgatgggcaagttcctgaccctttctaggtctgtttctatgagcggggggagctacactagaaaactgagggggctccttctaggtctgtaattcatctaggactccccccgagggttgagctccacatgaggaggctctatagaggtggtatctcgatagaacatccttttctttagataggtggttagcagtggtggcaacttgctgactacaggagagataaactgtctattagaaaaataggtctaggccgggcacggtggttcacgcctataatcccagcactttgggaggccaaggcaggtggattacctgaggtcaggagttcaagaccagcctggccaacatggtgaaaccccgtctctactaaaaatacaaaaattagccgggcgtggtggcatacccctgtaatcccagctactcaggacgctgaggcaggagaattgcttgagcccgggaggcagaggttgcagtaagttgagatcatgccactgcactccagcctggctgacagagcgagactctgtctcaaaaaaaaaaaaaagaaatgggtctagatttcaaaacacgacaaagaaaacttagaagagtttgagataacaaggaaggaaagtagtgtttaaagaggtagacttttttttttttttgagacagagttttgctcttgttgaccaggctggagtacagtggtgcgatctcggctcactgcaacctttgacttccagtttcaagcgattctcctgcctcggcctcctgagtagctgggattacaggcacccaccaccacacccagctaatttttgtatttttaatagagacagggtttcaccatgttggccaggctggtctcgaactcctgaccttacgatccacccaccttggcctcccaaagtgctgggattacaggtgttagccaccacacctggccaagaggtagacatttttagggaactgagcagctcagagcaggtttagacatggagagagatctagaaggcttagtgacttactagatgaccctgggcaagtccttgcttatctttggttttgctttcctgcttctaccataatggggtatttctctgggtttatttctgatgttctggtcacgtgtgattctgcgtggaatgccagactagtagttgggttcctggggttattgatgaagatcaggtcaaggtgctacaggtggaccagtagtatcaaaggaaggacagcattgggtgggggtcacaggagagacctgatcctgctgtgtgcagtttgcagtggtctggagccaaggacagactgtctccccattgcatgagaatgggaaccagagttgggaggcatgatcccctgctgtttccttgccttttataccctcagctcttgtggtaataaaccattcatcctgtgatcatccacttgagacctgtgttcatattattctcttagcctgagtatcccttccctattgagtctcacttgtcaggctctacctgtccttcagaaccccactcaaatttcaacttattcagcaacaacaacaaatatttattgagcaactacaaagtgccaggaactgtgttagacactggagatacaacagaaaatgaggaaaatgataagagccctgtgctatggagctcacagtctggtcagagaaatgggcatcagaaagtaaacaaaaatatggccatttactgtggctcgtacctgtaatcccagcactttgggaggcctaggtaggtggattgcatgagctcaggagttcaagaccagcctgggcaacatggcaaaaccccatctctacaaataatacaaaaattagctgggtgtggtggcgtgcacctgtagtcccagctacttgggaggctgaggagggaggatcacttgagcccaggaggtagaagttgcagtgagccaagattgcgctgttgcactccagtctgggtgacagagcaagacccacgtctcaaaagaaaaaaaaagtaaacaaaaataggaaaaaaaaattgggatttgtgtgtgtatgtttgtgtgcgtgtgcgtgtatgtgtgcatgtgtgtgttttagtctcaggtaactgctttcaatgaaacaactgggtaaaaagagaattatgggaaatccacattaaatagagtggacagggaagccttctctgaaaaggtgacattgagctgagatgtaaggatggtaaggatccagctatgcatgggaaaagccgaaaggaagggggtttcaggttgagggaaaagcagtgcaggccctgaggagggaaagagctttgtgatttgaggaatgacaggcctgtgtgagtagaatggcagagactaggagtcagggatggtacaaggttgaaaaagtagacaggagccagctcctgaaggatcttgaaggccatggtagggagtatggaacacagtgggaagctgagcacgtagacaaatgttctacccttacaccttctattgtttcccacagattgggggattcttgcctttgcaggggctcacagtctggcacaatgatacataactacaacatatcacacctggctcacaaggatgttagaatgatcctgggtgataatgagggtgaagatacaaatcatgatacctggcacctaatggatggatgttcagtaaacgtcagctgaagtaaaataaagtcgaattccttttgtcttcttccctctgcagaattgggaagcacgtctactgctggagaggtcacatgctgccaagtgcccagacattgccacccagctggctgggactaagaaggtgcagcaggagctaagcaggccgggcatgctggagatgttgctccctggccagcctgaggctgtggcccgcctccgcgccacctttgctggcctctactcactggatgtggtacgtgggcagcctgtttctcctaccacaggcctcctaggtggcagagacctacagcccaatgtgttggggagggtggagctggcattgtgacaaggggaaggtggagctggcaaggttggtgatgctctggagaacccctagaactctgagcagaagggcagcctcataatggaaggatgggggctggaatccattgtaagctccctcagcaaaggtagagatgaggatggcaaccagagggaagggactaaggcaggtggcaagaattgagaagtgtatcaggctgcctgctgcagagccctgagctgttgctaaagaaaggcctgttctcattgcatcggctgctgcagggggtttgttgggagtgtcatccagatagtagcatcctgcctgaaggaatttgtggctgttctccctcctgctcttcctctgatgctgctctgcataaccagctggacctaagcttcttgcctctttagcctttaaacttttgataactgctttctgcctcctgccagggtgaagaaggggaccaggccatcgccgaggcccttgctgcccctagccggtttgtgctaaagccccagagagagggtggaggtaggtggatctccctttgcagggctcctcaatgagagggactagcaggctgtggccagtgctcattggcacttactctgggcacagtcccgggcatgggggaaactattggaactgacacaggccacatgttggacagtgtcccctaagaccctgtgaccaagtccgggagcacaggggaatctgattaaccagcattgaagggtttggacaagttttacctgaggtgcctgtgggtagattgttgggaagtagagtagggtcatattaggagactggagagaatacatgtctgttttcctttctagtttgaaactccttgaggtcaggggtcatgtctgcctctccagaggagaggatttttttaatctttgtcttaagaggtgggtaggaatttcccaggtggaaaggaggaagagtgttccatacaaaagggacaacctcaagccaaggcaccgggccatgaaagtgtgagatgtttggaggttaatgagaaactggtgaggctggagggggagctgggaggggacagggatttaggctggaaaaatggtttgcatcctgattataaagggccttgaatatatactgagaaattggattttatcttaagggcagtgggaagccattagggagttttaagccaggaagggacacattgatccaggactcaagtggttagcagtggtgggaacttgcaaaacttacagtttctgcattgtagaagatgtcctggaatgaggggagacactggaagcagaaagaccgtggaagaggctgatacagttgttcagaagagcaacgtagaggcctgggctagggctatgactatggggccaactggagagacatgtcctagatagtgagagggtagtggaagggaggagttaaatatgactcaggggtaccttttgcctgattgggagtaggaaggtccaggaggggcaggttcaggcagaagtaataagttctgcttggacaagttgagtttgtttgggggccagtcatatgatgtctaagcagggagcctgcattaaatatttggaagttaacaattttttttttttttttgagacggattctcgctctgtcaccaggctggagtgcagtggcatgatcttggctcactgcaaccactgcctcccaggttcaagcgattctcctgcctcagcctcctgagtagctgggactacaggtgtgcgccaccacacccagctaatttttgtatttttagtagagatggggtttcatcatattggccaggatggtctcaatctcttgacctcatgatctgcctgcctcggcctcccaaagtgctgggattacaggcgtgagccaccatgcccggccggaagttaacaatttttagggtatagatggagactcaggaataggagagatctccttgggaaaatgtacatgggggagagagcaagcgtggaggaccaattcccctgggaccccagcatttaagagaaggagccagcaatggagcttgagaaggaacagctgtaggtaggaggagaaccagggcagaacagtgtagtggaagatgtgttcactgcatgagtaagggctctcctgtcaaagtgagcttccctcctgagaagccagatatgccctggcttcactgagcgggtgccaggaactgaggctgctgacttgcccatgtggccccaaaagtgagggcatgggatggaggaggtaggcagagggtccagggtgactggccagtttcattgcaggtaacaacctatatggggaggaaatggtacaggccctgaaacagctgaaggacagtgaggagagggcctcctacatcctcatggagaagatcgaacctgagccttttgagaattgcctgctacggcctggcagccctgcccgagtggtccagtgcatttcagagctgggcatctttggggtctatgtcaggtgagccaatcaggagaagctctttccactacctgcttgcaagagtgccagccaagtgagccagcctagaggggaacactggaaagagtcaggaatcctgggcttcggtgccagctctgccaatcactagctttattacctgtttctttatctattaaatgaggccaaggacccaagacctgcccaccttaccagggtatcagatgaagccctgatgagaagtcctttgcaaccgtgaaggaaactccaaatagcaccaagaggactcagaacacatggtttgacaacctaggactagaaggagactccagagaggcatagagactctaaaatcctagcactttcttggtatagacagttacccaggtactgctcagctgggtccagggaaggtcctgggtttggggctgagtccaggtgatgtgtgtcccctgcctccatttctataggcaggaaaagacactcgtgatgaacaagcacgtggggcatctacttcgaaccaaagccatcgagcatgcagatggtggtgtggcagcgggagtggcagtcctggacaacccataccctgtgtgagggcacaaccaggccacgggaccttctatcctctgtatttgtcattcctctcctagccctcctgaggggtatcctcctaaagacctccaaagtttttatggaagggtaaatactggtaccttcccccagctttccatctgaggaccagaaaagttgtgtctcccttagatgagatctagacgcccccaaatccttgagatgtgggtatagctcagggtaagctgctctgaggtaaaggtccatgaaccctgccccactcctgtcagcccctcatcagccttttcagcaggttccagtgcctgacttgggataggactgagtggtaggaggagggggagtggaggggcatagcctttccctaattctgccttaaataaaactgcattgctgattcagtgatgattccttacttcgtgcatagaggggaggcgggagctgtaatctacgttagcccacttaagatgtattagagcagggaagtgactggtctgtaatcagggtccccctagaccagtctctacaggtggaaccctgaagtttcaatccttagccacccactaatgctcttactggatcacagggaggaatgagagtccctggcaggagcccaggagggaaggcaaccaagatgggacatacataacagttgtgaactggcttcagtcactttcctgcttagctcaggggcttgtcaaaggccctgtcagtgaagcctccttcgctctgcccaaaccaaaagttctagaaggaagatattggggatagtcctaggaaatacccctcccttcccatctgccacacaaatcagagccactaatgaatatacagcctcagggcacagatacctaagaaaacaagtcaccacttcttgagatcacaggctttattcctacaaccacagggcttgagcctgactggggcaagaaaacagagtttcatctgagaatgtctcttatgggctgggttctgttcaggggagggtgggaacagaggacaaggaagacaagctcctctggccctaggaacaaaacacatttactccttcaaagaagcagatgatctgaataccctctggagactgaatctgcccatacagcccctggagccaatgggcagacagtactggcatctggcacaaaagggaattcagacccagaacagaagcagcaaaatattttaaaaatagtaaattgttcctggactcacaaatcattgtttttaagggcaagtgcatgcccaatataagtactggggcttcctaagagagctgacataggattacacagctgcctccctgcttcagtggaggccctcacatcccctttgaacacttaacttgggtaggagaggtagccttttcgtctctgttctgggttctgagagctctgcagtctggaggcacagcagactgaggctgacctgggccctgtcctttctgcctggcagtcacaggatgttgtctctacctggagacaaagctggtttccggtcccagacagctggtcaagggagggtagtgtgggtcaacactggccctcagcactcctgagggggcaaagaggatgggcaaagtttggagcaggaggaatccta

SEQ ID NO: 20 - DNA, 30 bp, Artificial
  Primer/Probe
acgttggatgcagatctggtaaccaccatc

SEQ ID NO: 21 - DNA, 30 bp, Artificial
  Primer/Probe
acgttggatgagttctctgacgcatttccg

SEQ ID NO: 22 - DNA, 31 bp, Artificial
  Primer/Probe
acgttggatgctttctagatttttcacccag

SEQ ID NO: 23 - DNA, 30 bp, Artificial
  Primer/Probe
acgttggatgaggatgaactgctagccaac

SEQ ID NO: 24 - DNA, 30 bp, Artificial
  Primer/Probe
acgttggatgccctcttctagcttcacttg

SEQ ID NO: 25 - DNA, 30 bp, Artificial
  Primer/Probe
acgttggatgaaacactaggaaccttaatc

SEQ ID NO: 26 - DNA, 31 bp, Artificial
  Primer/Probe
acgttggatggatcataagcttttgtcttac

SEQ ID NO: 27 - DNA, 30 bp, Artificial
  Primer/Probe
acgttggatgctgtatttttatcactgtcc

SEQ ID NO: 28 - DNA, 30 bp, Artificial
  Primer/Probe
acgttggatgtaacctctagttggttctgc

SEQ ID NO: 29 - DNA, 30 bp, Artificial
  Primer/Probe
acgttggatgggagttgagtgtcattccag

SEQ ID NO: 30 - DNA, 30 bp, Artificial
  Primer/Probe
acgttggatgcttaccttcctgaattgagg

SEQ ID NO: 31 - DNA, 30 bp, Artificial
  Primer/Probe
acgttggatgaatttccctctggaaggatg

SEQ ID NO: 32 - DNA, 30 bp, Artificial
  Primer/Probe
acgttggatgtgatgctcagagtcacacac

SEQ ID NO: 33 - DNA, 30 bp, Artificial
  Primer/Probe
acgttggatgcctactgttatgaagcaccc

SEQ ID NO: 34 - DNA, 30 bp, Artificial
  Primer/Probe
acgttggatgcaggacttctctttctccag

SEQ ID NO: 35 - DNA, 30 bp, Artificial
  Primer/Probe
acgttggatgttatcctgggtgactacctc

SEQ ID NO: 36 - DNA, 30 bp, Artificial
  Primer/Probe
acgttggatgtagaccagtctctacaggtg

SEQ ID NO: 37 - DNA, 30 bp, Artificial
  Primer/Probe
acgttggatgtctcattcctccctgtgatc

SEQ ID NO: 38 - DNA, 30 bp, Artificial
  Primer/Probe
acgttggatgacggttgcaaaggacttctc

SEQ ID NO: 39 - DNA, 30 bp, Artificial
  Primer/Probe
acgttggatgttaaatgaggccaaggaccc

SEQ ID NO: 40 - DNA, 30 bp, Artificial
  Primer/Probe
acgttggatgccagtgagagctgattgttg

SEQ ID NO: 41 - DNA, 30 bp, Artificial
  Primer/Probe
acgttggatggaatcctcaggaatccacag

SEQ ID NO: 42 - DNA, 30 bp, Artificial
  Primer/Probe
acgttggatgttgcgttttcaccttcaccc

SEQ ID NO: 43 - DNA, 30 bp, Artificial
  Primer/Probe
acgttggatgtttccactgcttaaagcagc

SEQ ID NO: 44 - DNA, 30 bp, Artificial
  Primer/Probe
acgttggatgtctgagaatcagctgagcac

SEQ ID NO: 45 - DNA, 30 bp, Artificial
  Primer/Probe
acgttggatgcagcccagcatattccaacc

SEQ ID NO: 46 - DNA, 30 bp, Artificial
  Primer/Probe
acgttggatgctgtgcaatcttccagttcc

SEQ ID NO: 47 - DNA, 30 bp, Artificial
  Primer/Probe
acgttggatgcaaactctttccaggtaggg

SEQ ID NO: 48 - DNA, 30 bp, Artificial
  Primer/Probe
acgttggatgcgaggtgatgactggtatag

SEQ ID NO: 49 - DNA, 30 bp, Artificial
  Primer/Probe
acgttggatgtctttctccaatgaagagcc

SEQ ID NO: 50 - DNA, 31 bp, Artificial
  Primer/Probe
acgttggatgcttttgcctctaatgctttcc

SEQ ID NO: 51 - DNA, 30 bp, Artificial
  Primer/Probe
acgttggatgaagtcccagaaaaatccccc

SEQ ID NO: 52 - DNA, 17 bp, Artificial
  Primer/Probe
caccatctttccggctc

SEQ ID NO: 53 - DNA, 23 bp, Artificial
  Primer/Probe
cagtattttcaaaatttgggaat

SEQ ID NO: 54 - DNA, 21 bp, Artificial
  Primer/Probe
cttttactagtaggaaaggaa

SEQ ID NO: 55 - DNA, 24 bp, Artificial
  Primer/Probe
cttttgtcttacaaaaaggtattt

SEQ ID NO: 56 - DNA, 17 bp, Artificial
  Primer/Probe
ttggttctgctccttcc

SEQ ID NO: 57 - DNA, 20 bp, Artificial
  Primer/Probe
aattcatcaggaaagcctca

SEQ ID NO: 58 - DNA, 20 bp, Artificial
  Primer/Probe
aaacattgttcaaaggacta

SEQ ID NO: 59 - DNA, 17 bp, Artificial
  Primer/Probe
gtccccctccctctaga

SEQ ID NO: 60 - DNA, 17 bp, Artificial
  Primer/Probe
atccttagccacccact

SEQ ID NO: 61 - DNA, 17 bp, Artificial
  Primer/Probe
tcatctgataccctggt

SEQ ID NO: 62 - DNA, 19 bp, Artificial
  Primer/Probe
tctggaaacagtgtaaatg

SEQ ID NO: 63 - DNA, 23 bp, Artificial
  Primer/Probe
ccctgccattaaaaattttttca

SEQ ID NO: 64 - DNA, 17 bp, Artificial
  Primer/Probe
ctcccatcacattcctg

SEQ ID NO: 65 - DNA, 17 bp, Artificial
  Primer/Probe
gcagctcctggcccccc

SEQ ID NO: 66 - DNA, 17 bp, Artificial
  Primer/Probe
ttgaacccatgtctctg

SEQ ID NO: 67 - DNA, 17 bp, Artificial
  Primer/Probe
taatgctttccctgctg